SEQUENCE LISTING (318 sequences)

SEQ ID NO: 1 - protein, 1137 aa, unidentified
  source 1..1137: note = Mucor ambiguus
MTKYAHDQIPGPEPHYLLGNVPDIFPDSLGNLIKLHDKYGPIVHLSMGGHELLSVDDPAVLETICEDNEYFTKEIESVYSDLAILNGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLSAGHNTTSAFLSWTMLELCKHPEVVENIKQEIANCGIKAGEVPTPEQVKECKYLDLVIKESLRIHPPITSILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 2 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgaatggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgagcgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccagcattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 3 - protein, 1137 aa, synthetic construct
  REGION 1..1137: note = Synthetic
MTKYAHDQIPGPEPHYLLGNVPDIFPDSLGNLIKLHDKYGPIVHLSMGGHELLSVDDPAVLETICEDNEYFTKEIESVYSDLAILNGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLNAGHNTTSAFLSWTMLELCKHPEVVENIKQEIANCGIKAGEVPTPEQVKECKYLDLVIKESLRIHPPITSILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 4 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgaatggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgaacgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccagcattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 5 - protein, 1137 aa, synthetic construct
  REGION 1..1137: note = Synthetic
MTKYAHDQIPGPEPHYLLGNVPDIFPDSLGNLIKLHDKYGPIVHLSMGGHELLSVDDPAVLETICEDNEYFTKEIESVYSDLAILNGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLTAGHNTTSAFLSWTMLELCKHPEVVENIKQEIANCGIKAGEVPTPEQVKECKYLDLVIKESLRIHPPITSILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 6 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgaatggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgaccgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccagcattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 7 - protein, 1137 aa, synthetic construct
  REGION 1..1137: note = Synthetic
MTKYAHDQIPGPEPHYLLGNVPDIFPDSLGNLIKLHDKYGPIVHLSMGGHELLSVDDPAVLETICEDNEYFTKEIESVYSDLAILNGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLSAGHTTTSAFLSWTMLELCKHPEVVENIKQEIANCGIKAGEVPTPEQVKECKYLDLVIKESLRIHPPITSILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 8 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgaatggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgagcgcaggtcatactaccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccagcattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 9 - protein, 1137 aa, synthetic construct
  REGION 1..1137: note = Synthetic
MTKYAHDQIPGPEPHYLLGNVPDIFPDSLGNLIKLHDKYGPIVHLSMGGHELLSVDDPAVLETICEDNEYFTKEIESVYSDLAILMGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLNAGHNTTSAFLSWTMLELCKHPEVVENIKQEIANCGIKAGEVPTPEQVKECKYLDLVIKESLRIHPPITDILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 10 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgatgggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgaacgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattaccgacattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 11 - protein, 1137 aa, synthetic construct
  REGION 1..1137: note = Synthetic
MTKYAHDQIPGPEPHYLLGNVPDIFPDSLGNLIKLHDKYGPIVHLSMGGHELLSVDDPAVLETICEDNEYFTKEIESVYSDLAILFGRGLVTTSTADPDWQLGHKLIMNAFSARAMKAYHYKMGESISELCEIMDSFAKSGEDFDVSRWFIALALESIGKIGFDYDFDLLKDPNAPRHPFTVALAYVQSMIMKRASTLSWLKWYQTTTNVRFHRDLQTLRGTVEEVLKDRREHPHTEADQSDLLDFMIKAESKEGEKLNDSLIRDNIITFLTAGHNTTSAFLSWTMLELCKHPEVVENIKQEIANCGIKAGEVPTPEQVKECKYLDLVIKESLRIHPPITHILKYCKKDATVKASNGDEYDIKAGQLLQVNINALHHNPKVWEDPDVFNPDRFSGDTDNLPNTAWLTFSTGPRACIGRQFALQEGKLALVMILSRFHFKMDDPSQKIGYAVVVSTKPVGFFAKIESSQLPEPTEEIVVTKRRESKAVPQEKVKPAEFPLPPVTFLFGTQTNTSEEYARKLSGQAKEMGFKEVTVQDLDDWKLVKGEAIAKAQHDADAPSSEDDVKVSELVVVVTATYNGFPPDDANEFAKWLDERTKDSEATKNNMLSGMLYAVFGCGNRDWTSTFQKFPKKVDSGFELLGGERLLPAGEGDASDDIDGDFSLWSASFWTALMQRYGQSSSGKNADIMSNNGPAADPSQDFTLEFINIVKEKVKTEQAALNCNQLETVATIVENRELQHTEKSHRSTRHIQVQFDKSVDGKPLYEAGDHLEVVPVNEDRLVEIIATNLGLVLDSVFEVKDLDIKNLSPRSVAANIKGPCTIRNALKYYADLTGPPTRFSLSILSKQLKDSRPDIAERLQKALQPGKETERLKEFLASHRTLIDIIQAFKIKELNFKEFISSVNCIVPRKYSISSGPLEHPFDPSISVGVVDTVGGPDGNTHYFGLASGYLSHQEPGTKINAQIKACKSTFRLPDDPSTPVIFIAAGTGFSPFRGFLQERHAKGLKSSKKNSNGESSECYMFFGCRHPDQDFIYKEEFDAYLEDGTITELYTTFSRSGEVVKYVQHALLKHANLLYKLMEESNAKVYICGSAGSMAKDVKRTWERLLVQMSGVSESEAAEQIQAWVDEGKYNEDVWGT

SEQ ID NO: 12 - DNA, 3414 bp, synthetic construct
  misc_feature 1..3414: note = Synthetic
  source 1..3414: mol_type = other DNA
atgaccaaatatgcccatgatcagattccgggtcctgaaccgcattatctgctgggtaatgttccggatatttttccggatagcctgggcaatctgattaaactgcatgataaatatggtccgattgtgcatctgagcatgggtggtcatgaactgctgagcgttgatgatccggcagttctggaaaccatttgtgaagataatgaatatttcaccaaagaaatcgaaagcgtgtatagcgatctggcaattctgtttggtcgtggtctggttaccaccagtaccgcagatccggattggcagctgggtcataaactgattatgaatgcatttagcgcacgtgccatgaaagcctatcactataaaatgggtgaaagcattagcgaactgtgcgaaattatggatagctttgcaaaaagcggtgaggattttgatgttagccgttggtttattgcactggcactggaaagcattggtaaaatcggtttcgattatgatttcgacctgctgaaagatccgaatgcaccgcgtcatccgtttaccgttgcgctggcctatgttcagagtatgatcatgaaacgtgcaagcaccctgagctggctgaaatggtatcagaccaccaccaatgttcgttttcatcgtgatctgcagaccctgcgtggcaccgttgaagaagttctgaaagaccgtcgtgaacatccgcataccgaagcagatcagagcgatctgctggattttatgattaaagccgaaagcaaagaaggcgagaaactgaacgatagcctgattcgtgataacatcattacctttctgaccgcaggtcataataccacctcagcatttctgagctggaccatgctggaactgtgtaaacatccggaagttgtcgaaaacatcaaacaagaaattgccaactgcggtattaaagcgggtgaagttccgacaccggaacaggttaaagaatgtaaatatctggacctggtgatcaaagaaagcctgcgtattcatccgcctattacccacattctgaaatactgtaaaaaagacgcaaccgtgaaagccagcaatggtgatgaatatgatattaaagcaggtcagctgctgcaggttaacattaatgcactgcatcataacccgaaagtttgggaagatcctgatgtttttaacccggatcgttttagcggtgataccgataatctgccgaataccgcatggctgacctttagcaccggtccgcgtgcatgtattggtcgtcagtttgcactgcaagaaggtaaactggccctggttatgattctgagccgttttcatttcaaaatggatgatccgagccagaaaattggttatgcagttgttgttagcaccaaaccggttggtttttttgccaaaattgaaagcagccagctgccggaaccgaccgaagaaattgttgttaccaaacgtcgtgaaagtaaagcagttccgcaagaaaaagttaaaccggcagaatttccgctgcctccggtgacctttctgtttggcacccagaccaataccagcgaagaatatgcacgtaaactgagcggtcaggcaaaagaaatgggttttaaagaagttaccgtccaggatctggatgattggaaactggttaaaggtgaagcaattgcaaaagcacagcatgatgccgatgcaccgagcagcgaagatgatgttaaagtgagcgaactggttgttgttgtgaccgcaacctataatggttttccgcctgatgatgcaaacgaatttgcaaaatggctggatgaacgtaccaaagatagcgaagcaaccaaaaataacatgctgagcggtatgctgtatgcagtgtttggttgtggtaatcgtgattggaccagcacctttcagaaattcccgaaaaaagttgatagcggctttgaactgttaggtggtgaacgtctgctgccagccggtgaaggtgatgcaagtgatgatattgatggtgattttagcctgtggtctgccagcttttggaccgcactgatgcagcgttatggtcagagcagcagcggtaaaaatgcagatattatgagcaataatggtccggcagcagatccgagtcaggattttaccctggaatttatcaacatcgtgaaagagaaggtcaaaaccgaacaggcagcactgaattgtaatcagctggaaaccgttgcaaccattgttgaaaatcgcgaactgcagcatacagaaaaaagccatcgtagcacccgtcatattcaggttcagtttgataaaagcgtggatggtaaaccgctgtatgaagccggtgatcatctggaagtggttccggttaatgaagatcgtctggttgaaattattgccaccaatctgggtttagttctggatagcgtttttgaggtgaaagacctggatattaagaatctgagtccgcgtagcgttgcagcaaacattaaaggtccgtgtaccattcgtaatgccctgaaatattacgcagatctgaccggtcctccgacacgttttagtctgagcattctgtcaaaacagctgaaagacagccgtcctgatattgcagaacgcctgcagaaagcactgcagcctggtaaagaaaccgaacgtctgaaagaatttctggcaagtcatcgtaccctgatcgatattattcaggccttcaaaatcaaagaactgaacttcaaagagtttatcagcagcgttaattgcatcgttccgcgtaaatatagcattagcagtggtccgctggaacatccgtttgatccgagtattagcgttggtgttgttgataccgttggtggtccggatggtaatacccattattttggtctggcaagcggttatctgagccatcaagaaccgggtacaaaaatcaatgcacagattaaagcatgcaagagtacctttcgtctgccggatgatcctagcacaccggttatctttattgcagcaggcaccggttttagcccgtttcgtggttttctgcaagaacgtcatgcaaaaggtctgaaaagcagcaaaaaaaacagcaatggcgaaagcagcgagtgctatatgttttttggctgtcgtcatccggatcaggatttcatctataaagaagaatttgacgcctacctggaagatggcaccattacagaactgtataccacctttagccgtagcggtgaagttgttaaatatgttcagcacgcactgctgaaacatgcaaatctgctgtacaaactgatggaagaaagcaacgccaaagtgtatatttgtggtagcgcaggtagcatggcaaaagatgttaaacgtacctgggagcgcctgctggttcagatgagcggtgttagcgaaagcgaagcagcagagcagattcaggcatgggttgatgaaggcaaatataacgaagatgtttggggcacctaa

SEQ ID NO: 13 - DNA, 52 bp, synthetic construct
  misc_feature 1..52: note = Synthetic
  source 1..52: mol_type = other DNA
atcgctatacacgctttcgatttctgcggtgaaatattcattatcttcacaa

SEQ ID NO: 14 - DNA, 52 bp, synthetic construct
  misc_feature 1..52: note = Synthetic
  source 1..52: mol_type = other DNA
ttgtgaagataatgaatatttcaccgcagaaatcgaaagcgtgtatagcgat

SEQ ID NO: 15 - DNA, 57 bp, synthetic construct
  misc_feature 1..57: note = Synthetic
  source 1..57: mol_type = other DNA
cagatcgctatacacgctttcgatttcgcaggtgaaatattcattatcttcacaaat

SEQ ID NO: 16 - DNA, 57 bp, synthetic construct
  misc_feature 1..57: note = Synthetic
  source 1..57: mol_type = other DNA
atttgtgaagataatgaatatttcacctgcgaaatcgaaagcgtgtatagcgatctg

SEQ ID NO: 17 - DNA, 51 bp, synthetic construct
  misc_feature 1..51: note = Synthetic
  source 1..51: mol_type = other DNA
atcgctatacacgctttcgatttcatcggtgaaatattcattatcttcaca

SEQ ID NO: 18 - DNA, 51 bp, synthetic construct
  misc_feature 1..51: note = Synthetic
  source 1..51: mol_type = other DNA
tgtgaagataatgaatatttcaccgatgaaatcgaaagcgtgtatagcgat

SEQ ID NO: 19 - DNA, 52 bp, synthetic construct
  misc_feature 1..52: note = Synthetic
  source 1..52: mol_type = other DNA
atcgctatacacgctttcgatttctccggtgaaatattcattatcttcacaa

SEQ ID NO: 20 - DNA, 52 bp, synthetic construct
  misc_feature 1..52: note = Synthetic
  source 1..52: mol_type = other DNA
ttgtgaagataatgaatatttcaccggagaaatcgaaagcgtgtatagcgat

SEQ ID NO: 21 - DNA, 52 bp, synthetic construct
  misc_feature 1..52: note = Synthetic
  source 1..52: mol_type = other DNA
atcgctatacacgctttcgatttctaaggtgaaatattcattatcttcacaa

SEQ ID NO: 22 - DNA, 52 bp, synthetic construct
  misc_feature 1..52: note = Synthetic
  source 1..52: mol_type = other DNA
ttgtgaagataatgaatatttcaccttagaaatcgaaagcgtgtatagcgat

SEQ ID NO: 23 - DNA, 47 bp, synthetic construct
  misc_feature 1..47: note = Synthetic
  source 1..47: mol_type = other DNA
tcgctatacacgctttcgatttcattggtgaaatattcattatcttc

SEQ ID NO: 24 - DNA, 47 bp, synthetic construct
  misc_feature 1..47: note = Synthetic
  source 1..47: mol_type = other DNA
gaagataatgaatatttcaccaatgaaatcgaaagcgtgtatagcga

SEQ ID NO: 25 - DNA, 51 bp, synthetic construct
  misc_feature 1..51: note = Synthetic
  source 1..51: mol_type = other DNA
atcgctatacacgctttcgatttcctgggtgaaatattcattatcttcaca

SEQ ID NO: 26 - DNA, 51 bp, synthetic construct
  misc_feature 1..51: note = Synthetic
  source 1..51: mol_type = other DNA
tgtgaagataatgaatatttcacccaggaaatcgaaagcgtgtatagcgat

SEQ ID NO: 27 - DNA, 49 bp, synthetic construct
  misc_feature 1..49: note = Synthetic
  source 1..49: mol_type = other DNA
atacacgctttcgatttctctggtgaaatattcattatcttcacaaatg

SEQ ID NO: 28 - DNA, 49 bp, synthetic construct
  misc_feature 1..49: note = Synthetic
  source 1..49: mol_type = other DNA
catttgtgaagataatgaatatttcaccagagaaatcgaaagcgtgtat

SEQ ID NO: 29 - DNA, 52 bp, synthetic construct
  misc_feature 1..52: note = Synthetic
  source 1..52: mol_type = other DNA
gatcgctatacacgctttcgatttcgctggtgaaatattcattatcttcaca

SEQ ID NO: 30 - DNA, 52 bp, synthetic construct
  misc_feature 1..52: note = Synthetic
  source 1..52: mol_type = other DNA
tgtgaagataatgaatatttcaccagcgaaatcgaaagcgtgtatagcgatc

SEQ ID NO: 31 - DNA, 52 bp, synthetic construct
  misc_feature 1..52: note = Synthetic
  source 1..52: mol_type = other DNA
gatcgctatacacgctttcgatttccgtggtgaaatattcattatcttcaca

SEQ ID NO: 32 - DNA, 52 bp, synthetic construct
  misc_feature 1..52: note = Synthetic
  source 1..52: mol_type = other DNA
tgtgaagataatgaatatttcaccacggaaatcgaaagcgtgtatagcgatc

SEQ ID NO: 33 - DNA, 51 bp, synthetic construct
  misc_feature 1..51: note = Synthetic
  source 1..51: mol_type = other DNA
atcgctatacacgctttcgatttcataggtgaaatattcattatcttcaca

SEQ ID NO: 34 - DNA, 51 bp, synthetic construct
  misc_feature 1..51: note = Synthetic
  source 1..51: mol_type = other DNA
tgtgaagataatgaatatttcacctatgaaatcgaaagcgtgtatagcgat

SEQ ID NO: 35 - DNA, 42 bp, synthetic construct
  misc_feature 1..42: note = Synthetic
  source 1..42: mol_type = other DNA
tcagaattgccagatcgctagccacgctttcgatttctttgg

SEQ ID NO: 36 - DNA, 42 bp, synthetic construct
  misc_feature 1..42: note = Synthetic
  source 1..42: mol_type = other DNA
ccaaagaaatcgaaagcgtggctagcgatctggcaattctga

SEQ ID NO: 37 - DNA, 39 bp, synthetic construct
  misc_feature 1..39: note = Synthetic
  source 1..39: mol_type = other DNA
cagaattgccagatcgctacacacgctttcgatttcttt

SEQ ID NO: 38 - DNA, 39 bp, synthetic construct
  misc_feature 1..39: note = Synthetic
  source 1..39: mol_type = other DNA
aaagaaatcgaaagcgtgtgtagcgatctggcaattctg

SEQ ID NO: 39 - DNA, 39 bp, synthetic construct
  misc_feature 1..39: note = Synthetic
  source 1..39: mol_type = other DNA
agaattgccagatcgctatccacgctttcgatttctttg

SEQ ID NO: 40 - DNA, 39 bp, synthetic construct
  misc_feature 1..39: note = Synthetic
  source 1..39: mol_type = other DNA
caaagaaatcgaaagcgtggatagcgatctggcaattct

SEQ ID NO: 41 - DNA, 42 bp, synthetic construct
  misc_feature 1..42: note = Synthetic
  source 1..42: mol_type = other DNA
tcagaattgccagatcgctacccacgctttcgatttctttgg

SEQ ID NO: 42 - DNA, 42 bp, synthetic construct
  misc_feature 1..42: note = Synthetic
  source 1..42: mol_type = other DNA
ccaaagaaatcgaaagcgtgggtagcgatctggcaattctga

SEQ ID NO: 43 - DNA, 43 bp, synthetic construct
  misc_feature 1..43: note = Synthetic
  source 1..43: mol_type = other DNA
ttcagaattgccagatcgctcttcacgctttcgatttctttgg

SEQ ID NO: 44 - DNA, 43 bp, synthetic construct
  misc_feature 1..43: note = Synthetic
  source 1..43: mol_type = other DNA
ccaaagaaatcgaaagcgtgaagagcgatctggcaattctgaa

SEQ ID NO: 45 - DNA, 49 bp, synthetic construct
  misc_feature 1..49: note = Synthetic
  source 1..49: mol_type = other DNA
gaattgccagatcgcttaacacgctttcgatttctttggtgaaatattc

SEQ ID NO: 46 - DNA, 49 bp, synthetic construct
  misc_feature 1..49: note = Synthetic
  source 1..49: mol_type = other DNA
gaatatttcaccaaagaaatcgaaagcgtgttaagcgatctggcaattc

SEQ ID NO: 47 - DNA, 39 bp, synthetic construct
  misc_feature 1..39: note = Synthetic
  source 1..39: mol_type = other DNA
agaattgccagatcgctattcacgctttcgatttctttg

SEQ ID NO: 48 - DNA, 39 bp, synthetic construct
  misc_feature 1..39: note = Synthetic
  source 1..39: mol_type = other DNA
caaagaaatcgaaagcgtgaatagcgatctggcaattct

SEQ ID NO: 49 - DNA, 43 bp, synthetic construct
  misc_feature 1..43: note = Synthetic
  source 1..43: mol_type = other DNA
ttcagaattgccagatcgctctgcacgctttcgatttctttgg

SEQ ID NO: 50 - DNA, 43 bp, synthetic construct
  misc_feature 1..43: note = Synthetic
  source 1..43: mol_type = other DNA
ccaaagaaatcgaaagcgtgcagagcgatctggcaattctgaa

SEQ ID NO: 51 - DNA, 42 bp, synthetic construct
  misc_feature 1..42: note = Synthetic
  source 1..42: mol_type = other DNA
tcagaattgccagatcgctacgcacgctttcgatttctttgg

SEQ ID NO: 52 - DNA, 42 bp, synthetic construct
  misc_feature 1..42: note = Synthetic
  source 1..42: mol_type = other DNA
ccaaagaaatcgaaagcgtgcgtagcgatctggcaattctga

SEQ ID NO: 53 - DNA, 42 bp, synthetic construct
  misc_feature 1..42: note = Synthetic
  source 1..42: mol_type = other DNA
tcagaattgccagatcgctactcacgctttcgatttctttgg

SEQ ID NO: 54 - DNA, 42 bp, synthetic construct
  misc_feature 1..42: note = Synthetic
  source 1..42: mol_type = other DNA
ccaaagaaatcgaaagcgtgagtagcgatctggcaattctga

SEQ ID NO: 55 - DNA, 42 bp, synthetic construct
  misc_feature 1..42: note = Synthetic
  source 1..42: mol_type = other DNA
tcagaattgccagatcgctagtcacgctttcgatttctttgg

SEQ ID NO: 56 - DNA, 42 bp, synthetic construct
  misc_feature 1..42: note = Synthetic
  source 1..42: mol_type = other DNA
ccaaagaaatcgaaagcgtgactagcgatctggcaattctga

SEQ ID NO: 57 - DNA, 42 bp, synthetic construct
  misc_feature 1..42: note = Synthetic
  source 1..42: mol_type = other DNA
gaccattcagaattgccgcatcgctatacacgctttcgattt

SEQ ID NO: 58 - DNA, 42 bp, synthetic construct
  misc_feature 1..42: note = Synthetic
  source 1..42: mol_type = other DNA
aaatcgaaagcgtgtatagcgatgcggcaattctgaatggtc

SEQ ID NO: 59 - DNA, 46 bp, synthetic construct
  misc_feature 1..46: note = Synthetic
  source 1..46: mol_type = other DNA
cacgaccattcagaattgcatcatcgctatacacgctttcgatttc

SEQ ID NO: 60 - DNA, 46 bp, synthetic construct
  misc_feature 1..46: note = Synthetic
  source 1..46: mol_type = other DNA
gaaatcgaaagcgtgtatagcgatgatgcaattctgaatggtcgtg

SEQ ID NO: 61 - DNA, 35 bp, synthetic construct
  misc_feature 1..35: note = Synthetic
  source 1..35: mol_type = other DNA
gaccattcagaattgcctgatcgctatacacgctt

SEQ ID NO: 62 - DNA, 35 bp, synthetic construct
  misc_feature 1..35: note = Synthetic
  source 1..35: mol_type = other DNA
aagcgtgtatagcgatcaggcaattctgaatggtc

SEQ ID NO: 63 - DNA, 40 bp, synthetic construct
  misc_feature 1..40: note = Synthetic
  source 1..40: mol_type = other DNA
gaccacgaccattcgcaattgccagatcgctatacacgct

SEQ ID NO: 64 - DNA, 40 bp, synthetic construct
  misc_feature 1..40: note = Synthetic
  source 1..40: mol_type = other DNA
agcgtgtatagcgatctggcaattgcgaatggtcgtggtc

SEQ ID NO: 65 - DNA, 45 bp, synthetic construct
  misc_feature 1..45: note = Synthetic
  source 1..45: mol_type = other DNA
gtaaccagaccacgaccattatcaattgccagatcgctatacacg

SEQ ID NO: 66 - DNA, 45 bp, synthetic construct
  misc_feature 1..45: note = Synthetic
  source 1..45: mol_type = other DNA
cgtgtatagcgatctggcaattgataatggtcgtggtctggttac

SEQ ID NO: 67 - DNA, 33 bp, synthetic construct
  misc_feature 1..33: note = Synthetic
  source 1..33: mol_type = other DNA
cagaccacgaccattctgaattgccagatcgct

SEQ ID NO: 68 - DNA, 33 bp, synthetic construct
  misc_feature 1..33: note = Synthetic
  source 1..33: mol_type = other DNA
agcgatctggcaattcagaatggtcgtggtctg

SEQ ID NO: 69 - DNA, 39 bp, synthetic construct
  misc_feature 1..39: note = Synthetic
  source 1..39: mol_type = other DNA
gtaaccagaccacgaccagccagaattgccagatcgcta

SEQ ID NO: 70 - DNA, 39 bp, synthetic construct
  misc_feature 1..39: note = Synthetic
  source 1..39: mol_type = other DNA
tagcgatctggcaattctggctggtcgtggtctggttac

SEQ ID NO: 71 - DNA, 39 bp, synthetic construct
  misc_feature 1..39: note = Synthetic
  source 1..39: mol_type = other DNA
gtaaccagaccacgaccacacagaattgccagatcgcta

SEQ ID NO: 72 - DNA, 39 bp, synthetic construct
  misc_feature 1..39: note = Synthetic
  source 1..39: mol_type = other DNA
tagcgatctggcaattctgtgtggtcgtggtctggttac

SEQ ID NO: 73 - DNA, 33 bp, synthetic construct
  misc_feature 1..33: note = Synthetic
  source 1..33: mol_type = other DNA
accagaccacgaccatccagaattgccagatcg

SEQ ID NO: 74 - DNA, 33 bp, synthetic construct
  misc_feature 1..33: note = Synthetic
  source 1..33: mol_type = other DNA
cgatctggcaattctggatggtcgtggtctggt

SEQ ID NO: 75 - DNA, 37 bp, synthetic construct
  misc_feature 1..37: note = Synthetic
  source 1..37: mol_type = other DNA
gtaaccagaccacgaccctccagaattgccagatcgc

SEQ ID NO: 76 - DNA, 37 bp, synthetic construct
  misc_feature 1..37: note = Synthetic
  source 1..37: mol_type = other DNA
gcgatctggcaattctggagggtcgtggtctggttac

SEQ ID NO: 77 - DNA, 39 bp, synthetic construct
  misc_feature 1..39: note = Synthetic
  source 1..39: mol_type = other DNA
gtaaccagaccacgaccaaacagaattgccagatcgcta

SEQ ID NO: 78 - DNA, 39 bp, synthetic construct
  misc_feature 1..39: note = Synthetic
  source 1..39: mol_type = other DNA
tagcgatctggcaattctgtttggtcgtggtctggttac

SEQ ID NO: 79 - DNA, 39 bp, synthetic construct
  misc_feature 1..39: note = Synthetic
  source 1..39: mol_type = other DNA
gtaaccagaccacgaccacccagaattgccagatcgcta

SEQ ID NO: 80 - DNA, 39 bp, synthetic construct
  misc_feature 1..39: note = Synthetic
  source 1..39: mol_type = other DNA
tagcgatctggcaattctgggtggtcgtggtctggttac

SEQ ID NO: 81 - DNA, 33 bp, synthetic construct
  misc_feature 1..33: note = Synthetic
  source 1..33: mol_type = other DNA
accagaccacgaccatgcagaattgccagatcg

SEQ ID NO: 82 - DNA, 33 bp, synthetic construct
  misc_feature 1..33: note = Synthetic
  source 1..33: mol_type = other DNA
cgatctggcaattctgcatggtcgtggtctggt

SEQ ID NO: 83 - DNA, 36 bp, synthetic construct
  misc_feature 1..36: note = Synthetic
  source 1..36: mol_type = other DNA
agaccacgaccaatcagaattgccagatcgctatac

SEQ ID NO: 84 - DNA, 36 bp, synthetic construct
  misc_feature 1..36: note = Synthetic
  source 1..36: mol_type = other DNA
gtatagcgatctggcaattctgattggtcgtggtct

SEQ ID NO: 85 - DNA, 34 bp, synthetic construct
  misc_feature 1..34: note = Synthetic
  source 1..34: mol_type = other DNA
aaccagaccacgacccttcagaattgccagatcg

SEQ ID NO: 86 - DNA, 34 bp, synthetic construct
  misc_feature 1..34: note = Synthetic
  source 1..34: mol_type = other DNA
cgatctggcaattctgaagggtcgtggtctggtt

SEQ ID NO: 87 - DNA, 43 bp, synthetic construct
  misc_feature 1..43: note = Synthetic
  source 1..43: mol_type = other DNA
gtggtaaccagaccacgacctagcagaattgccagatcgctat

SEQ ID NO: 88 - DNA, 43 bp, synthetic construct
  misc_feature 1..43: note = Synthetic
  source 1..43: mol_type = other DNA
atagcgatctggcaattctgctaggtcgtggtctggttaccac

SEQ ID NO: 89 - DNA, 40 bp, synthetic construct
  misc_feature 1..40: note = Synthetic
  source 1..40: mol_type = other DNA
ccagaccacgacccatcagaattgccagatcgctatacac

SEQ ID NO: 90 - DNA, 40 bp, synthetic construct
  misc_feature 1..40: note = Synthetic
  source 1..40: mol_type = other DNA
gtgtatagcgatctggcaattctgatgggtcgtggtctgg

SEQ ID NO: 91 - DNA, 39 bp, synthetic construct
  misc_feature 1..39: note = Synthetic
  source 1..39: mol_type = other DNA
gtaaccagaccacgaccaggcagaattgccagatcgcta

SEQ ID NO: 92 - DNA, 39 bp, synthetic construct
  misc_feature 1..39: note = Synthetic
  source 1..39: mol_type = other DNA
tagcgatctggcaattctgcctggtcgtggtctggttac

SEQ ID NO: 93 - DNA, 37 bp, synthetic construct
  misc_feature 1..37: note = Synthetic
  source 1..37: mol_type = other DNA
gtaaccagaccacgaccctgcagaattgccagatcgc

SEQ ID NO: 94 - DNA, 37 bp, synthetic construct
  misc_feature 1..37: note = Synthetic
  source 1..37: mol_type = other DNA
gcgatctggcaattctgcagggtcgtggtctggttac

SEQ ID NO: 95 - DNA, 40 bp, synthetic construct
  misc_feature 1..40: note = Synthetic
  source 1..40: mol_type = other DNA
ccagaccacgacccctcagaattgccagatcgctatacac

SEQ ID NO: 96 - DNA, 40 bp, synthetic construct
  misc_feature 1..40: note = Synthetic
  source 1..40: mol_type = other DNA
gtgtatagcgatctggcaattctgaggggtcgtggtctgg

SEQ ID NO: 97 - DNA, 36 bp, synthetic construct
  misc_feature 1..36: note = Synthetic
  source 1..36: mol_type = other DNA
agaccacgaccactcagaattgccagatcgctatac

SEQ ID NO: 98 - DNA, 36 bp, synthetic construct
  misc_feature 1..36: note = Synthetic
  source 1..36: mol_type = other DNA
gtatagcgatctggcaattctgagtggtcgtggtct

SEQ ID NO: 99 - DNA, 36 bp, synthetic construct
  misc_feature 1..36: note = Synthetic
  source 1..36: mol_type = other DNA
agaccacgaccagtcagaattgccagatcgctatac

SEQ ID NO: 100 - DNA, 36 bp, synthetic construct
  misc_feature 1..36: note = Synthetic
  source 1..36: mol_type = other DNA
gtatagcgatctggcaattctgactggtcgtggtct

SEQ ID NO: 101 - DNA, 39 bp, synthetic construct
  misc_feature 1..39: note = Synthetic
  source 1..39: mol_type = other DNA
gtaaccagaccacgaccaaccagaattgccagatcgcta

SEQ ID NO: 102 - DNA, 39 bp, synthetic construct
  misc_feature 1..39: note = Synthetic
  source 1..39: mol_type = other DNA
tagcgatctggcaattctggttggtcgtggtctggttac

SEQ ID NO: 103 - DNA, 43 bp, synthetic construct
  misc_feature 1..43: note = Synthetic
  source 1..43: mol_type = other DNA
gtggtaaccagaccacgaccccacagaattgccagatcgctat

SEQ ID NO: 104 - DNA, 43 bp, synthetic construct
  misc_feature 1..43: note = Synthetic
  source 1..43: mol_type = other DNA
atagcgatctggcaattctgtggggtcgtggtctggttaccac

SEQ ID NO: 105 - DNA, 33 bp, synthetic construct
  misc_feature 1..33: note = Synthetic
  source 1..33: mol_type = other DNA
accagaccacgaccatacagaattgccagatcg

SEQ ID NO: 106 - DNA, 33 bp, synthetic construct
  misc_feature 1..33: note = Synthetic
  source 1..33: mol_type = other DNA
cgatctggcaattctgtatggtcgtggtctggt

SEQ ID NO: 107 - DNA, 29 bp, synthetic construct
  misc_feature 1..29: note = Synthetic
  source 1..29: mol_type = other DNA
cggtactggtggtagccagaccacgacca

SEQ ID NO: 108 - DNA, 29 bp, synthetic construct
  misc_feature 1..29: note = Synthetic
  source 1..29: mol_type = other DNA
tggtcgtggtctggctaccaccagtaccg

SEQ ID NO: 109 - DNA, 29 bp, synthetic construct
  misc_feature 1..29: note = Synthetic
  source 1..29: mol_type = other DNA
cggtactggtggtatccagaccacgacca

SEQ ID NO: 110 - DNA, 29 bp, synthetic construct
  misc_feature 1..29: note = Synthetic
  source 1..29: mol_type = other DNA
tggtcgtggtctggataccaccagtaccg

SEQ ID NO: 111 - DNA, 29 bp, synthetic construct
  misc_feature 1..29: note = Synthetic
  source 1..29: mol_type = other DNA
cggtactggtggtacccagaccacgacca

SEQ ID NO: 112 - DNA, 29 bp, synthetic construct
  misc_feature 1..29: note = Synthetic
  source 1..29: mol_type = other DNA
tggtcgtggtctgggtaccaccagtaccg

SEQ ID NO: 113 - DNA, 31 bp, synthetic construct
  misc_feature 1..31: note = Synthetic
  source 1..31: mol_type = other DNA
cggtactggtggtaagcagaccacgaccatt

SEQ ID NO: 114 - DNA, 31 bp, synthetic construct
  misc_feature 1..31: note = Synthetic
  source 1..31: mol_type = other DNA
aatggtcgtggtctgcttaccaccagtaccg

SEQ ID NO: 115 - DNA, 39 bp, synthetic construct
  misc_feature 1..39: note = Synthetic
  source 1..39: mol_type = other DNA
atctgcggtactggtggtctgcagaccacgaccattcag

SEQ ID NO: 116 - DNA, 39 bp, synthetic construct
  misc_feature 1..39: note = Synthetic
  source 1..39: mol_type = other DNA
ctgaatggtcgtggtctgcagaccaccagtaccgcagat

SEQ ID NO: 117 - DNA, 36 bp, synthetic construct
  misc_feature 1..36: note = Synthetic
  source 1..36: mol_type = other DNA
ctgcggtactggtggtactcagaccacgaccattca

SEQ ID NO: 118 - DNA, 36 bp, synthetic construct
  misc_feature 1..36: note = Synthetic
  source 1..36: mol_type = other DNA
tgaatggtcgtggtctgagtaccaccagtaccgcag

SEQ ID NO: 119 - DNA, 36 bp, synthetic construct
  misc_feature 1..36: note = Synthetic
  source 1..36: mol_type = other DNA
ctgcggtactggtggtagtcagaccacgaccattca

SEQ ID NO: 120 - DNA, 36 bp, synthetic construct
  misc_feature 1..36: note = Synthetic
  source 1..36: mol_type = other DNA
tgaatggtcgtggtctgactaccaccagtaccgcag

SEQ ID NO: 121 - DNA, 29 bp, synthetic construct
  misc_feature 1..29: note = Synthetic
  source 1..29: mol_type = other DNA
cggtactggtggcaaccagaccacgacca

SEQ ID NO: 122 - DNA, 29 bp, synthetic construct
  misc_feature 1..29: note = Synthetic
  source 1..29: mol_type = other DNA
tggtcgtggtctggttgccaccagtaccg

SEQ ID NO: 123 - DNA, 38 bp, synthetic construct
  misc_feature 1..38: note = Synthetic
  source 1..38: mol_type = other DNA
gatctgcggtactggtgtcaaccagaccacgaccattc

SEQ ID NO: 124 - DNA, 38 bp, synthetic construct
  misc_feature 1..38: note = Synthetic
  source 1..38: mol_type = other DNA
gaatggtcgtggtctggttgacaccagtaccgcagatc

SEQ ID NO: 125 - DNA, 32 bp, synthetic construct
  misc_feature 1..32: note = Synthetic
  source 1..32: mol_type = other DNA
ctgcggtactggtgccaaccagaccacgacca

SEQ ID NO: 126 - DNA, 32 bp, synthetic construct
  misc_feature 1..32: note = Synthetic
  source 1..32: mol_type = other DNA
tggtcgtggtctggttggcaccagtaccgcag

SEQ ID NO: 127 - DNA, 44 bp, synthetic construct
  misc_feature 1..44: note = Synthetic
  source 1..44: mol_type = other DNA
tctgcggtactggttagaaccagaccacgaccattcagaattgc

SEQ ID NO: 128 - DNA, 44 bp, synthetic construct
  misc_feature 1..44: note = Synthetic
  source 1..44: mol_type = other DNA
gcaattctgaatggtcgtggtctggttctaaccagtaccgcaga

SEQ ID NO: 129 - DNA, 44 bp, synthetic construct
  misc_feature 1..44: note = Synthetic
  source 1..44: mol_type = other DNA
tctgcggtactggtctgaaccagaccacgaccattcagaattgc

SEQ ID NO: 130 - DNA, 44 bp, synthetic construct
  misc_feature 1..44: note = Synthetic
  source 1..44: mol_type = other DNA
gcaattctgaatggtcgtggtctggttcagaccagtaccgcaga

SEQ ID NO: 131 - DNA, 31 bp, synthetic construct
  misc_feature 1..31: note = Synthetic
  source 1..31: mol_type = other DNA
cggtactggtgctaaccagaccacgaccatt

SEQ ID NO: 132 - DNA, 31 bp, synthetic construct
  misc_feature 1..31: note = Synthetic
  source 1..31: mol_type = other DNA
aatggtcgtggtctggttagcaccagtaccg

SEQ ID NO: 133 - DNA, 38 bp, synthetic construct
  misc_feature 1..38: note = Synthetic
  source 1..38: mol_type = other DNA
atactctgaacataggccgccgcaacggtaaacggatg

SEQ ID NO: 134 - DNA, 38 bp, synthetic construct
  misc_feature 1..38: note = Synthetic
  source 1..38: mol_type = other DNA
catccgtttaccgttgcggcggcctatgttcagagtat

SEQ ID NO: 135 - DNA, 45 bp, synthetic construct
  misc_feature 1..45: note = Synthetic
  source 1..45: mol_type = other DNA
gatcatactctgaacataggcatccgcaacggtaaacggatgacg

SEQ ID NO: 136 - DNA, 45 bp, synthetic construct
  misc_feature 1..45: note = Synthetic
  source 1..45: mol_type = other DNA
cgtcatccgtttaccgttgcggatgcctatgttcagagtatgatc

SEQ ID NO: 137 - DNA, 31 bp, synthetic construct
  misc_feature 1..31: note = Synthetic
  source 1..31: mol_type = other DNA
ctctgaacataggcctgcgcaacggtaaacg

SEQ ID NO: 138 - DNA, 31 bp, synthetic construct
  misc_feature 1..31: note = Synthetic
  source 1..31: mol_type = other DNA
cgtttaccgttgcgcaggcctatgttcagag

SEQ ID NO: 139 - DNA, 40 bp, synthetic construct
  misc_feature 1..40: note = Synthetic
  source 1..40: mol_type = other DNA
gtttcatgatcatactcgcaacataggccagcgcaacggt

SEQ ID NO: 140 - DNA, 40 bp, synthetic construct
  misc_feature 1..40: note = Synthetic
  source 1..40: mol_type = other DNA
accgttgcgctggcctatgttgcgagtatgatcatgaaac

SEQ ID NO: 141 - DNA, 41 bp, synthetic construct
  misc_feature 1..41: note = Synthetic
  source 1..41: mol_type = other DNA
cgtttcatgatcatactatcaacataggccagcgcaacggt

SEQ ID NO: 142 - DNA, 41 bp, synthetic construct
  misc_feature 1..41: note = Synthetic
  source 1..41: mol_type = other DNA
accgttgcgctggcctatgttgatagtatgatcatgaaacg

SEQ ID NO: 143 - DNA, 37 bp, synthetic construct
  misc_feature 1..37: note = Synthetic
  source 1..37: mol_type = other DNA
gtttcatgatcatactttgaacataggccagcgcaac

SEQ ID NO: 144 - DNA, 37 bp, synthetic construct
  misc_feature 1..37: note = Synthetic
  source 1..37: mol_type = other DNA
gttgcgctggcctatgttcaaagtatgatcatgaaac

SEQ ID NO: 145 - DNA, 40 bp, synthetic construct
  misc_feature 1..40: note = Synthetic
  source 1..40: mol_type = other DNA
ggtgcttgcacgtttcatggccatactctgaacataggcc

SEQ ID NO: 146 - DNA, 40 bp, synthetic construct
  misc_feature 1..40: note = Synthetic
  source 1..40: mol_type = other DNA
ggcctatgttcagagtatggccatgaaacgtgcaagcacc

SEQ ID NO: 147 - DNA, 40 bp, synthetic construct
  misc_feature 1..40: note = Synthetic
  source 1..40: mol_type = other DNA
ggtgcttgcacgtttcatgtccatactctgaacataggcc

SEQ ID NO: 148 - DNA, 40 bp, synthetic construct
  misc_feature 1..40: note = Synthetic
  source 1..40: mol_type = other DNA
ggcctatgttcagagtatggacatgaaacgtgcaagcacc

SEQ ID NO: 149 - DNA, 45 bp, synthetic construct
  misc_feature 1..45: note = Synthetic
  source 1..45: mol_type = other DNA
cagggtgcttgcacgtttcatctgcatactctgaacataggccag

SEQ ID NO: 150 - DNA, 45 bp, synthetic construct
  misc_feature 1..45: note = Synthetic
  source 1..45: mol_type = other DNA
ctggcctatgttcagagtatgcagatgaaacgtgcaagcaccctg

SEQ ID NO: 151 - DNA, 40 bp, synthetic construct
  misc_feature 1..40: note = Synthetic
  source 1..40: mol_type = other DNA
acctgcgctcagaaaggtagcgatgttatcacgaatcagg

SEQ ID NO: 152 - DNA, 40 bp, synthetic construct
  misc_feature 1..40: note = Synthetic
  source 1..40: mol_type = other DNA
cctgattcgtgataacatcgctacctttctgagcgcaggt

SEQ ID NO: 153 - DNA, 40 bp, synthetic construct
  misc_feature 1..40: note = Synthetic
  source 1..40: mol_type = other DNA
acctgcgctcagaaaggtatcgatgttatcacgaatcagg

SEQ ID NO: 154 - DNA, 40 bp, synthetic construct
  misc_feature 1..40: note = Synthetic
  source 1..40: mol_type = other DNA
cctgattcgtgataacatcgatacctttctgagcgcaggt

SEQ ID NO: 155 - DNA, 45 bp, synthetic construct
  misc_feature 1..45: note = Synthetic
  source 1..45: mol_type = other DNA
atgacctgcgctcagaaaggtctggatgttatcacgaatcaggct

SEQ ID NO: 156 - DNA, 45 bp, synthetic construct
  misc_feature 1..45: note = Synthetic
  source 1..45: mol_type = other DNA
agcctgattcgtgataacatccagacctttctgagcgcaggtcat

SEQ ID NO: 157 - DNA, 36 bp, synthetic construct
  misc_feature 1..36: note = Synthetic
  source 1..36: mol_type = other DNA
cgctcagaaaggcaatgatgttatcacgaatcaggc

SEQ ID NO: 158 - DNA, 36 bp, synthetic construct
  misc_feature 1..36: note = Synthetic
  source 1..36: mol_type = other DNA
gcctgattcgtgataacatcattgcctttctgagcg

SEQ ID NO: 159 - DNA, 45 bp, synthetic construct
  misc_feature 1..45: note = Synthetic
  source 1..45: mol_type = other DNA
ttatgacctgcgctcagaaagtcaatgatgttatcacgaatcagg

SEQ ID NO: 160 - DNA, 45 bp, synthetic construct
  misc_feature 1..45: note = Synthetic
  source 1..45: mol_type = other DNA
cctgattcgtgataacatcattgactttctgagcgcaggtcataa

SEQ ID NO: 161 - DNA, 48 bp, synthetic construct
  misc_feature 1..48: note = Synthetic
  source 1..48: mol_type = other DNA
gacctgcgctcagaaactgaatgatgttatcacgaatcaggctatcgt

SEQ ID NO: 162 - DNA, 48 bp, synthetic construct
  misc_feature 1..48: note = Synthetic
  source 1..48: mol_type = other DNA
acgatagcctgattcgtgataacatcattcagtttctgagcgcaggtc

SEQ ID NO: 163 - DNA, 44 bp, synthetic construct
  misc_feature 1..44: note = Synthetic
  source 1..44: mol_type = other DNA
gaggtggtattatgacctgcggccagaaaggtaatgatgttatc

SEQ ID NO: 164 - DNA, 44 bp, synthetic construct
  misc_feature 1..44: note = Synthetic
  source 1..44: mol_type = other DNA
gataacatcattacctttctggccgcaggtcataataccacctc

SEQ ID NO: 165 - DNA, 39 bp, synthetic construct
  misc_feature 1..39: note = Synthetic
  source 1..39: mol_type = other DNA
gtggtattatgacctgcgcacagaaaggtaatgatgtta

SEQ ID NO: 166 - DNA, 39 bp, synthetic construct
  misc_feature 1..39: note = Synthetic
  source 1..39: mol_type = other DNA
taacatcattacctttctgtgcgcaggtcataataccac

SEQ ID NO: 167 - DNA, 44 bp, synthetic construct
  misc_feature 1..44: note = Synthetic
  source 1..44: mol_type = other DNA
gaggtggtattatgacctgcgtccagaaaggtaatgatgttatc

SEQ ID NO: 168 - DNA, 44 bp, synthetic construct
  misc_feature 1..44: note = Synthetic
  source 1..44: mol_type = other DNA
gataacatcattacctttctggacgcaggtcataataccacctc

SEQ ID NO: 169 - DNA, 49 bp, synthetic construct
  misc_feature 1..49: note = Synthetic
  source 1..49: mol_type = other DNA
gctgaggtggtattatgacctgcctccagaaaggtaatgatgttatcac

SEQ ID NO: 170 - DNA, 49 bp, synthetic construct
  misc_feature 1..49: note = Synthetic
  source 1..49: mol_type = other DNA
gtgataacatcattacctttctggaggcaggtcataataccacctcagc

SEQ ID NO: 171 - DNA, 44 bp, synthetic construct
  misc_feature 1..44: note = Synthetic
  source 1..44: mol_type = other DNA
gaggtggtattatgacctgcgaacagaaaggtaatgatgttatc

SEQ ID NO: 172 - DNA, 44 bp, synthetic construct
  misc_feature 1..44: note = Synthetic
  source 1..44: mol_type = other DNA
gataacatcattacctttctgttcgcaggtcataataccacctc

SEQ ID NO: 173 - DNA, 39 bp, synthetic construct
  misc_feature 1..39: note = Synthetic
  source 1..39: mol_type = other DNA
gtggtattatgacctgcgcccagaaaggtaatgatgtta

SEQ ID NO: 174 - DNA, 39 bp, synthetic construct
  misc_feature 1..39: note = Synthetic
  source 1..39: mol_type = other DNA
taacatcattacctttctgggcgcaggtcataataccac

SEQ ID NO: 175 - DNA, 44 bp, synthetic construct
  misc_feature 1..44: note = Synthetic
  source 1..44: mol_type = other DNA
gaggtggtattatgacctgcgtgcagaaaggtaatgatgttatc

SEQ ID NO: 176 - DNA, 44 bp, synthetic construct
  misc_feature 1..44: note = Synthetic
  source 1..44: mol_type = other DNA
gataacatcattacctttctgcacgcaggtcataataccacctc

SEQ ID NO: 177 - DNA, 45 bp, synthetic construct
  misc_feature 1..45: note = Synthetic
  source 1..45: mol_type = other DNA
gtattatgacctgcgatcagaaaggtaatgatgttatcacgaatc

SEQ ID NO: 178 - DNA, 45 bp, synthetic construct
  misc_feature 1..45: note = Synthetic
  source 1..45: mol_type = other DNA
gattcgtgataacatcattacctttctgatcgcaggtcataatac

SEQ ID NO: 179 - DNA, 46 bp, synthetic construct
  misc_feature 1..46: note = Synthetic
  source 1..46: mol_type = other DNA
ggtattatgacctgccttcagaaaggtaatgatgttatcacgaatc

SEQ ID NO: 180 - DNA, 46 bp, synthetic construct
  misc_feature 1..46: note = Synthetic
  source 1..46: mol_type = other DNA
gattcgtgataacatcattacctttctgaaggcaggtcataatacc

SEQ ID NO: 181 - DNA, 49 bp, synthetic construct
  misc_feature 1..49: note = Synthetic
  source 1..49: mol_type = other DNA
gctgaggtggtattatgacctgctagcagaaaggtaatgatgttatcac

SEQ ID NO: 182 - DNA, 49 bp, synthetic construct
  misc_feature 1..49: note = Synthetic
  source 1..49: mol_type = other DNA
gtgataacatcattacctttctgctagcaggtcataataccacctcagc

SEQ ID NO: 183 - DNA, 46 bp, synthetic construct
  misc_feature 1..46: note = Synthetic
  source 1..46: mol_type = other DNA
ggtattatgacctgccatcagaaaggtaatgatgttatcacgaatc

SEQ ID NO: 184 - DNA, 46 bp, synthetic construct
  misc_feature 1..46: note = Synthetic
  source 1..46: mol_type = other DNA
gattcgtgataacatcattacctttctgatggcaggtcataatacc

SEQ ID NO: 185 - DNA, 45 bp, synthetic construct
  misc_feature 1..45: note = Synthetic
  source 1..45: mol_type = other DNA
gtattatgacctgcgttcagaaaggtaatgatgttatcacgaatc

SEQ ID NO: 186 - DNA, 45 bp, synthetic construct
  misc_feature 1..45: note = Synthetic
  source 1..45: mol_type = other DNA
gattcgtgataacatcattacctttctgaacgcaggtcataatac

SEQ ID NO: 187 - DNA, 44 bp, synthetic construct
  misc_feature 1..44: note = Synthetic
  source 1..44: mol_type = other DNA
gaggtggtattatgacctgcgggcagaaaggtaatgatgttatc

SEQ ID NO: 188 - DNA, 44 bp, synthetic construct
  misc_feature 1..44: note = Synthetic
  source 1..44: mol_type = other DNA
gataacatcattacctttctgcccgcaggtcataataccacctc

SEQ ID NO: 189 - DNA, 49 bp, synthetic construct
  misc_feature 1..49: note = Synthetic
  source 1..49: mol_type = other DNA
gctgaggtggtattatgacctgcctgcagaaaggtaatgatgttatcac

SEQ ID NO: 190 - DNA, 49 bp, synthetic construct
  misc_feature 1..49: note = Synthetic
  source 1..49: mol_type = other DNA
gtgataacatcattacctttctgcaggcaggtcataataccacctcagc

SEQ ID NO: 191 - DNA, 37 bp, synthetic construct
  misc_feature 1..37: note = Synthetic
  source 1..37: mol_type = other DNA
ggtggtattatgacctgccctcagaaaggtaatgatg

SEQ ID NO: 192 - DNA, 37 bp, synthetic construct
  misc_feature 1..37: note = Synthetic
  source 1..37: mol_type = other DNA
catcattacctttctgagggcaggtcataataccacc

SEQ ID NO: 193 - DNA, 39 bp, synthetic construct
  misc_feature 1..39: note = Synthetic
  source 1..39: mol_type = other DNA
attatgacctgcggtcagaaaggtaatgatgttatcacg

SEQ ID NO: 194 - DNA, 39 bp, synthetic construct
  misc_feature 1..39: note = Synthetic
  source 1..39: mol_type = other DNA
cgtgataacatcattacctttctgaccgcaggtcataat

SEQ ID NO: 195 - DNA, 44 bp, synthetic construct
  misc_feature 1..44: note = Synthetic
  source 1..44: mol_type = other DNA
gaggtggtattatgacctgcgaccagaaaggtaatgatgttatc

SEQ ID NO: 196 - DNA, 44 bp, synthetic construct
  misc_feature 1..44: note = Synthetic
  source 1..44: mol_type = other DNA
gataacatcattacctttctggtcgcaggtcataataccacctc

SEQ ID NO: 197 - DNA, 43 bp, synthetic construct
  misc_feature 1..43: note = Synthetic
  source 1..43: mol_type = other DNA
gaggtggtattatgacctgcccacagaaaggtaatgatgttat

SEQ ID NO: 198 - DNA, 43 bp, synthetic construct
  misc_feature 1..43: note = Synthetic
  source 1..43: mol_type = other DNA
ataacatcattacctttctgtgggcaggtcataataccacctc

SEQ ID NO: 199 - DNA, 49 bp, synthetic construct
  misc_feature 1..49: note = Synthetic
  source 1..49: mol_type = other DNA
gctgaggtggtattatgacctgcatacagaaaggtaatgatgttatcac

SEQ ID NO: 200 - DNA, 49 bp, synthetic construct
  misc_feature 1..49: note = Synthetic
  source 1..49: mol_type = other DNA
gtgataacatcattacctttctgtatgcaggtcataataccacctcagc

SEQ ID NO: 201 - DNA, 42 bp, synthetic construct
  misc_feature 1..42: note = Synthetic
  source 1..42: mol_type = other DNA
gctgaggtggtattatgaccatcgctcagaaaggtaatgatg

SEQ ID NO: 202 - DNA, 42 bp, synthetic construct
  misc_feature 1..42: note = Synthetic
  source 1..42: mol_type = other DNA
catcattacctttctgagcgatggtcataataccacctcagc

SEQ ID NO: 203 - DNA, 35 bp, synthetic construct
  misc_feature 1..35: note = Synthetic
  source 1..35: mol_type = other DNA
aggtggtattatgacctccgctcagaaaggtaatg

SEQ ID NO: 204 - DNA, 35 bp, synthetic construct
  misc_feature 1..35: note = Synthetic
  source 1..35: mol_type = other DNA
cattacctttctgagcggaggtcataataccacct

SEQ ID NO: 205 - DNA, 51 bp, synthetic construct
  misc_feature 1..51: note = Synthetic
  source 1..51: mol_type = other DNA
aaatgctgaggtggtattatgaccctggctcagaaaggtaatgatgttatc

SEQ ID NO: 206 - DNA, 51 bp, synthetic construct
  misc_feature 1..51: note = Synthetic
  source 1..51: mol_type = other DNA
gataacatcattacctttctgagccagggtcataataccacctcagcattt

SEQ ID NO: 207 - DNA, 41 bp, synthetic construct
  misc_feature 1..41: note = Synthetic
  source 1..41: mol_type = other DNA
atgctgaggtggtagcatgacctgcgctcagaaaggtaatg

SEQ ID NO: 208 - DNA, 41 bp, synthetic construct
  misc_feature 1..41: note = Synthetic
  source 1..41: mol_type = other DNA
cattacctttctgagcgcaggtcatgctaccacctcagcat

SEQ ID NO: 209 - DNA, 41 bp, synthetic construct
  misc_feature 1..41: note = Synthetic
  source 1..41: mol_type = other DNA
atgctgaggtggtacaatgacctgcgctcagaaaggtaatg

SEQ ID NO: 210 - DNA, 41 bp, synthetic construct
  misc_feature 1..41: note = Synthetic
  source 1..41: mol_type = other DNA
cattacctttctgagcgcaggtcattgtaccacctcagcat

SEQ ID NO: 211 - DNA, 35 bp, synthetic construct
  misc_feature 1..35: note = Synthetic
  source 1..35: mol_type = other DNA
aaatgctgaggtggtatcatgacctgcgctcagaa

SEQ ID NO: 212 - DNA, 35 bp, synthetic construct
  misc_feature 1..35: note = Synthetic
  source 1..35: mol_type = other DNA
ttctgagcgcaggtcatgataccacctcagcattt

SEQ ID NO: 213 - DNA, 39 bp, synthetic construct
  misc_feature 1..39: note = Synthetic
  source 1..39: mol_type = other DNA
gaaatgctgaggtggtctcatgacctgcgctcagaaagg

SEQ ID NO: 214 - DNA, 39 bp, synthetic construct
  misc_feature 1..39: note = Synthetic
  source 1..39: mol_type = other DNA
cctttctgagcgcaggtcatgagaccacctcagcatttc

SEQ ID NO: 215 - DNA, 41 bp, synthetic construct
  misc_feature 1..41: note = Synthetic
  source 1..41: mol_type = other DNA
atgctgaggtggtaaaatgacctgcgctcagaaaggtaatg

SEQ ID NO: 216 - DNA, 41 bp, synthetic construct
  misc_feature 1..41: note = Synthetic
  source 1..41: mol_type = other DNA
cattacctttctgagcgcaggtcattttaccacctcagcat

SEQ ID NO: 217 - DNA, 41 bp, synthetic construct
  misc_feature 1..41: note = Synthetic
  source 1..41: mol_type = other DNA
atgctgaggtggtaccatgacctgcgctcagaaaggtaatg

SEQ ID NO: 218 - DNA, 41 bp, synthetic construct
  misc_feature 1..41: note = Synthetic
  source 1..41: mol_type = other DNA
cattacctttctgagcgcaggtcatggtaccacctcagcat

SEQ ID NO: 219 - DNA, 35 bp, synthetic construct
  misc_feature 1..35: note = Synthetic
  source 1..35: mol_type = other DNA
aaatgctgaggtggtatgatgacctgcgctcagaa

SEQ ID NO: 220 - DNA, 35 bp, synthetic construct
  misc_feature 1..35: note = Synthetic
  source 1..35: mol_type = other DNA
ttctgagcgcaggtcatcataccacctcagcattt

SEQ ID NO: 221 - DNA, 35 bp, synthetic construct
  misc_feature 1..35: note = Synthetic
  source 1..35: mol_type = other DNA
aaatgctgaggtggtaatatgacctgcgctcagaa

SEQ ID NO: 222 - DNA, 35 bp, synthetic construct
  misc_feature 1..35: note = Synthetic
  source 1..35: mol_type = other DNA
ttctgagcgcaggtcatattaccacctcagcattt

SEQ ID NO: 223 - DNA, 35 bp, synthetic construct
  misc_feature 1..35: note = Synthetic
  source 1..35: mol_type = other DNA
atgctgaggtggtcttatgacctgcgctcagaaag

SEQ ID NO: 224 - DNA, 35 bp, synthetic construct
  misc_feature 1..35: note = Synthetic
  source 1..35: mol_type = other DNA
ctttctgagcgcaggtcataagaccacctcagcat

SEQ ID NO: 225 - DNA, 45 bp, synthetic construct
  misc_feature 1..45: note = Synthetic
  source 1..45: mol_type = other DNA
tcagaaatgctgaggtggttagatgacctgcgctcagaaaggtaa

SEQ ID NO: 226 - DNA, 45 bp, synthetic construct
  misc_feature 1..45: note = Synthetic
  source 1..45: mol_type = other DNA
ttacctttctgagcgcaggtcatctaaccacctcagcatttctga

SEQ ID NO: 227 - DNA, 40 bp, synthetic construct
  misc_feature 1..40: note = Synthetic
  source 1..40: mol_type = other DNA
atgctgaggtggtcatatgacctgcgctcagaaaggtaat

SEQ ID NO: 228 - DNA, 40 bp, synthetic construct
  misc_feature 1..40: note = Synthetic
  source 1..40: mol_type = other DNA
attacctttctgagcgcaggtcatatgaccacctcagcat

SEQ ID NO: 229 - DNA, 41 bp, synthetic construct
  misc_feature 1..41: note = Synthetic
  source 1..41: mol_type = other DNA
atgctgaggtggtaggatgacctgcgctcagaaaggtaatg

SEQ ID NO: 230 - DNA, 41 bp, synthetic construct
  misc_feature 1..41: note = Synthetic
  source 1..41: mol_type = other DNA
cattacctttctgagcgcaggtcatcctaccacctcagcat

SEQ ID NO: 231 - DNA, 39 bp, synthetic construct
  misc_feature 1..39: note = Synthetic
  source 1..39: mol_type = other DNA
gaaatgctgaggtggtctgatgacctgcgctcagaaagg

SEQ ID NO: 232 - DNA, 39 bp, synthetic construct
  misc_feature 1..39: note = Synthetic
  source 1..39: mol_type = other DNA
cctttctgagcgcaggtcatcagaccacctcagcatttc

SEQ ID NO: 233 - DNA, 40 bp, synthetic construct
  misc_feature 1..40: note = Synthetic
  source 1..40: mol_type = other DNA
atgctgaggtggtcctatgacctgcgctcagaaaggtaat

SEQ ID NO: 234 - DNA, 40 bp, synthetic construct
  misc_feature 1..40: note = Synthetic
  source 1..40: mol_type = other DNA
attacctttctgagcgcaggtcataggaccacctcagcat

SEQ ID NO: 235 - DNA, 35 bp, synthetic construct
  misc_feature 1..35: note = Synthetic
  source 1..35: mol_type = other DNA
aaatgctgaggtggtactatgacctgcgctcagaa

SEQ ID NO: 236 - DNA, 35 bp, synthetic construct
  misc_feature 1..35: note = Synthetic
  source 1..35: mol_type = other DNA
ttctgagcgcaggtcatagtaccacctcagcattt

SEQ ID NO: 237 - DNA, 35 bp, synthetic construct
  misc_feature 1..35: note = Synthetic
  source 1..35: mol_type = other DNA
aaatgctgaggtggtagtatgacctgcgctcagaa

SEQ ID NO: 238 - DNA, 35 bp, synthetic construct
  misc_feature 1..35: note = Synthetic
  source 1..35: mol_type = other DNA
ttctgagcgcaggtcatactaccacctcagcattt

SEQ ID NO: 239 - DNA, 41 bp, synthetic construct
  misc_feature 1..41: note = Synthetic
  source 1..41: mol_type = other DNA
atgctgaggtggtaacatgacctgcgctcagaaaggtaatg

SEQ ID NO: 240 - DNA, 41 bp, synthetic construct
  misc_feature 1..41: note = Synthetic
  source 1..41: mol_type = other DNA
cattacctttctgagcgcaggtcatgttaccacctcagcat

SEQ ID NO: 241 - DNA, 45 bp, synthetic construct
  misc_feature 1..45: note = Synthetic
  source 1..45: mol_type = other DNA
tcagaaatgctgaggtggtccaatgacctgcgctcagaaaggtaa

SEQ ID NO: 242 - DNA, 45 bp, synthetic construct
  misc_feature 1..45: note = Synthetic
  source 1..45: mol_type = other DNA
ttacctttctgagcgcaggtcattggaccacctcagcatttctga

SEQ ID NO: 243 - DNA, 35 bp, synthetic construct
  misc_feature 1..35: note = Synthetic
  source 1..35: mol_type = other DNA
aaatgctgaggtggtataatgacctgcgctcagaa

SEQ ID NO: 244 - DNA, 35 bp, synthetic construct
  misc_feature 1..35: note = Synthetic
  source 1..35: mol_type = other DNA
ttctgagcgcaggtcattataccacctcagcattt

SEQ ID NO: 245 - DNA, 36 bp, synthetic construct
  misc_feature 1..36: note = Synthetic
  source 1..36: mol_type = other DNA
atgctgaggtggcattatgacctgcgctcagaaagg

SEQ ID NO: 246 - DNA, 36 bp, synthetic construct
  misc_feature 1..36: note = Synthetic
  source 1..36: mol_type = other DNA
cctttctgagcgcaggtcataatgccacctcagcat

SEQ ID NO: 247 - DNA, 41 bp, synthetic construct
  misc_feature 1..41: note = Synthetic
  source 1..41: mol_type = other DNA
cagaaatgctgaggtgtcattatgacctgcgctcagaaagg

SEQ ID NO: 248 - DNA, 41 bp, synthetic construct
  misc_feature 1..41: note = Synthetic
  source 1..41: mol_type = other DNA
cctttctgagcgcaggtcataatgacacctcagcatttctg

SEQ ID NO: 249 - DNA, 47 bp, synthetic construct
  misc_feature 1..47: note = Synthetic
  source 1..47: mol_type = other DNA
cagaaatgctgaggtctgattatgacctgcgctcagaaaggtaatga

SEQ ID NO: 250 - DNA, 47 bp, synthetic construct
  misc_feature 1..47: note = Synthetic
  source 1..47: mol_type = other DNA
tcattacctttctgagcgcaggtcataatcagacctcagcatttctg

SEQ ID NO: 251 - DNA, 40 bp, synthetic construct
  misc_feature 1..40: note = Synthetic
  source 1..40: mol_type = other DNA
gtatttcagaatgctggtagcaggcggatgaatacgcagg

SEQ ID NO: 252 - DNA, 40 bp, synthetic construct
  misc_feature 1..40: note = Synthetic
  source 1..40: mol_type = other DNA
cctgcgtattcatccgcctgctaccagcattctgaaatac

SEQ ID NO: 253 - DNA, 40 bp, synthetic construct
  misc_feature 1..40: note = Synthetic
  source 1..40: mol_type = other DNA
gtatttcagaatgctggtatcaggcggatgaatacgcagg

SEQ ID NO: 254 - DNA, 40 bp, synthetic construct
  misc_feature 1..40: note = Synthetic
  source 1..40: mol_type = other DNA
cctgcgtattcatccgcctgataccagcattctgaaatac

SEQ ID NO: 255 - DNA, 48 bp, synthetic construct
  misc_feature 1..48: note = Synthetic
  source 1..48: mol_type = other DNA
atttcagaatgctggtctgaggcggatgaatacgcaggctttctttga

SEQ ID NO: 256 - DNA, 48 bp, synthetic construct
  misc_feature 1..48: note = Synthetic
  source 1..48: mol_type = other DNA
tcaaagaaagcctgcgtattcatccgcctcagaccagcattctgaaat

SEQ ID NO: 257 - DNA, 49 bp, synthetic construct
  misc_feature 1..49: note = Synthetic
  source 1..49: mol_type = other DNA
tcttttttacagtatttcagaatggcggtaataggcggatgaatacgca

SEQ ID NO: 258 - DNA, 49 bp, synthetic construct
  misc_feature 1..49: note = Synthetic
  source 1..49: mol_type = other DNA
tgcgtattcatccgcctattaccgccattctgaaatactgtaaaaaaga

SEQ ID NO: 259 - DNA, 43 bp, synthetic construct
  misc_feature 1..43: note = Synthetic
  source 1..43: mol_type = other DNA
ttttacagtatttcagaatgcaggtaataggcggatgaatacg

SEQ ID NO: 260 - DNA, 43 bp, synthetic construct
  misc_feature 1..43: note = Synthetic
  source 1..43: mol_type = other DNA
cgtattcatccgcctattacctgcattctgaaatactgtaaaa

SEQ ID NO: 261 - DNA, 50 bp, synthetic construct
  misc_feature 1..50: note = Synthetic
  source 1..50: mol_type = other DNA
tcttttttacagtatttcagaatgtcggtaataggcggatgaatacgcag

SEQ ID NO: 262 - DNA, 50 bp, synthetic construct
  misc_feature 1..50: note = Synthetic
  source 1..50: mol_type = other DNA
ctgcgtattcatccgcctattaccgacattctgaaatactgtaaaaaaga

SEQ ID NO: 263 - DNA, 53 bp, synthetic construct
  misc_feature 1..53: note = Synthetic
  source 1..53: mol_type = other DNA
cgtcttttttacagtatttcagaatctcggtaataggcggatgaatacgcagg

SEQ ID NO: 264 - DNA, 53 bp, synthetic construct
  misc_feature 1..53: note = Synthetic
  source 1..53: mol_type = other DNA
cctgcgtattcatccgcctattaccgagattctgaaatactgtaaaaaagacg

SEQ ID NO: 265 - DNA, 50 bp, synthetic construct
  misc_feature 1..50: note = Synthetic
  source 1..50: mol_type = other DNA
tcttttttacagtatttcagaatgaaggtaataggcggatgaatacgcag

SEQ ID NO: 266 - DNA, 50 bp, synthetic construct
  misc_feature 1..50: note = Synthetic
  source 1..50: mol_type = other DNA
ctgcgtattcatccgcctattaccttcattctgaaatactgtaaaaaaga

SEQ ID NO: 267 - DNA, 43 bp, synthetic construct
  misc_feature 1..43: note = Synthetic
  source 1..43: mol_type = other DNA
ttttacagtatttcagaatgccggtaataggcggatgaatacg

SEQ ID NO: 268 - DNA, 43 bp, synthetic construct
  misc_feature 1..43: note = Synthetic
  source 1..43: mol_type = other DNA
cgtattcatccgcctattaccggcattctgaaatactgtaaaa

SEQ ID NO: 269 - DNA, 50 bp, synthetic construct
  misc_feature 1..50: note = Synthetic
  source 1..50: mol_type = other DNA
tcttttttacagtatttcagaatgtgggtaataggcggatgaatacgcag

SEQ ID NO: 270 - DNA, 50 bp, synthetic construct
  misc_feature 1..50: note = Synthetic
  source 1..50: mol_type = other DNA
ctgcgtattcatccgcctattacccacattctgaaatactgtaaaaaaga

SEQ ID NO: 271 - DNA, 47 bp, synthetic construct
  misc_feature 1..47: note = Synthetic
  source 1..47: mol_type = other DNA
cttttttacagtatttcagaatgatggtaataggcggatgaatacgc

SEQ ID NO: 272 - DNA, 47 bp, synthetic construct
  misc_feature 1..47: note = Synthetic
  source 1..47: mol_type = other DNA
gcgtattcatccgcctattaccatcattctgaaatactgtaaaaaag

SEQ ID NO: 273 - DNA, 49 bp, synthetic construct
  misc_feature 1..49: note = Synthetic
  source 1..49: mol_type = other DNA
gtcttttttacagtatttcagaatcttggtaataggcggatgaatacgc

SEQ ID NO: 274 - DNA, 49 bp, synthetic construct
  misc_feature 1..49: note = Synthetic
  source 1..49: mol_type = other DNA
gcgtattcatccgcctattaccaagattctgaaatactgtaaaaaagac

SEQ ID NO: 275 - DNA, 53 bp, synthetic construct
  misc_feature 1..53: note = Synthetic
  source 1..53: mol_type = other DNA
cgtcttttttacagtatttcagaattagggtaataggcggatgaatacgcagg

SEQ ID NO: 276 - DNA, 53 bp, synthetic construct
  misc_feature 1..53: note = Synthetic
  source 1..53: mol_type = other DNA
cctgcgtattcatccgcctattaccctaattctgaaatactgtaaaaaagacg

SEQ ID NO: 277 - DNA, 49 bp, synthetic construct
  misc_feature 1..49: note = Synthetic
  source 1..49: mol_type = other DNA
gtcttttttacagtatttcagaatcatggtaataggcggatgaatacgc

SEQ ID NO: 278 - DNA, 49 bp, synthetic construct
  misc_feature 1..49: note = Synthetic
  source 1..49: mol_type = other DNA
gcgtattcatccgcctattaccatgattctgaaatactgtaaaaaagac

SEQ ID NO: 279 - DNA, 47 bp, synthetic construct
  misc_feature 1..47: note = Synthetic
  source 1..47: mol_type = other DNA
cttttttacagtatttcagaatgttggtaataggcggatgaatacgc

SEQ ID NO: 280 - DNA, 47 bp, synthetic construct
  misc_feature 1..47: note = Synthetic
  source 1..47: mol_type = other DNA
gcgtattcatccgcctattaccaacattctgaaatactgtaaaaaag

SEQ ID NO: 281 - DNA, 49 bp, synthetic construct
  misc_feature 1..49: note = Synthetic
  source 1..49: mol_type = other DNA
tcttttttacagtatttcagaatgggggtaataggcggatgaatacgca

SEQ ID NO: 282 - DNA, 49 bp, synthetic construct
  misc_feature 1..49: note = Synthetic
  source 1..49: mol_type = other DNA
tgcgtattcatccgcctattacccccattctgaaatactgtaaaaaaga

SEQ ID NO: 283 - DNA, 53 bp, synthetic construct
  misc_feature 1..53: note = Synthetic
  source 1..53: mol_type = other DNA
cgtcttttttacagtatttcagaatctgggtaataggcggatgaatacgcagg

SEQ ID NO: 284 - DNA, 53 bp, synthetic construct
  misc_feature 1..53: note = Synthetic
  source 1..53: mol_type = other DNA
cctgcgtattcatccgcctattacccagattctgaaatactgtaaaaaagacg

SEQ ID NO: 285 - DNA, 46 bp, synthetic construct
  misc_feature 1..46: note = Synthetic
  source 1..46: mol_type = other DNA
tcttttttacagtatttcagaatcctggtaataggcggatgaatac

SEQ ID NO: 286 - DNA, 46 bp, synthetic construct
  misc_feature 1..46: note = Synthetic
  source 1..46: mol_type = other DNA
gtattcatccgcctattaccaggattctgaaatactgtaaaaaaga

SEQ ID NO: 287 - DNA, 45 bp, synthetic construct
  misc_feature 1..45: note = Synthetic
  source 1..45: mol_type = other DNA
ttttttacagtatttcagaatggtggtaataggcggatgaatacg

SEQ ID NO: 288 - DNA, 45 bp, synthetic construct
  misc_feature 1..45: note = Synthetic
  source 1..45: mol_type = other DNA
cgtattcatccgcctattaccaccattctgaaatactgtaaaaaa

SEQ ID NO: 289 - DNA, 50 bp, synthetic construct
  misc_feature 1..50: note = Synthetic
  source 1..50: mol_type = other DNA
tcttttttacagtatttcagaatgacggtaataggcggatgaatacgcag

SEQ ID NO: 290 - DNA, 50 bp, synthetic construct
  misc_feature 1..50: note = Synthetic
  source 1..50: mol_type = other DNA
ctgcgtattcatccgcctattaccgtcattctgaaatactgtaaaaaaga

SEQ ID NO: 291 - DNA, 48 bp, synthetic construct
  misc_feature 1..48: note = Synthetic
  source 1..48: mol_type = other DNA
tcttttttacagtatttcagaatccaggtaataggcggatgaatacgc

SEQ ID NO: 292 - DNA, 48 bp, synthetic construct
  misc_feature 1..48: note = Synthetic
  source 1..48: mol_type = other DNA
gcgtattcatccgcctattacctggattctgaaatactgtaaaaaaga

SEQ ID NO: 293 - DNA, 53 bp, synthetic construct
  misc_feature 1..53: note = Synthetic
  source 1..53: mol_type = other DNA
cgtcttttttacagtatttcagaatataggtaataggcggatgaatacgcagg

SEQ ID NO: 294 - DNA, 53 bp, synthetic construct
  misc_feature 1..53: note = Synthetic
  source 1..53: mol_type = other DNA
cctgcgtattcatccgcctattacctatattctgaaatactgtaaaaaagacg

SEQ ID NO: 295 - DNA, 48 bp, synthetic construct
  misc_feature 1..48: note = Synthetic
  source 1..48: mol_type = other DNA
cgtcttttttacagtatttcagagcgctggtaataggcggatgaatac

SEQ ID NO: 296 - DNA, 48 bp, synthetic construct
  misc_feature 1..48: note = Synthetic
  source 1..48: mol_type = other DNA
gtattcatccgcctattaccagcgctctgaaatactgtaaaaaagacg

SEQ ID NO: 297 - DNA, 48 bp, synthetic construct
  misc_feature 1..48: note = Synthetic
  source 1..48: mol_type = other DNA
cgtcttttttacagtatttcagatcgctggtaataggcggatgaatac

SEQ ID NO: 298 - DNA, 48 bp, synthetic construct
  misc_feature 1..48: note = Synthetic
  source 1..48: mol_type = other DNA
gtattcatccgcctattaccagcgatctgaaatactgtaaaaaagacg

SEQ ID NO: 299 - DNA, 51 bp, synthetic construct
  misc_feature 1..51: note = Synthetic
  source 1..51: mol_type = other DNA
tgcgtcttttttacagtatttcagctggctggtaataggcggatgaatacg

SEQ ID NO: 300 - DNA, 51 bp, synthetic construct
  misc_feature 1..51: note = Synthetic
  source 1..51: mol_type = other DNA
cgtattcatccgcctattaccagccagctgaaatactgtaaaaaagacgca

SEQ ID NO: 301 - DNA, 46 bp, synthetic construct
  misc_feature 1..46: note = Synthetic
  source 1..46: mol_type = other DNA
gtcttttttacagtatttcagaacgctggtaataggcggatgaata

SEQ ID NO: 302 - DNA, 46 bp, synthetic construct
  misc_feature 1..46: note = Synthetic
  source 1..46: mol_type = other DNA
tattcatccgcctattaccagcgttctgaaatactgtaaaaaagac

SEQ ID NO: 303 - DNA, 41 bp, synthetic construct
  misc_feature 1..41: note = Synthetic
  source 1..41: mol_type = other DNA
caaccggtttggtgctaacagcaactgcataaccaattttc

SEQ ID NO: 304 - DNA, 41 bp, synthetic construct
  misc_feature 1..41: note = Synthetic
  source 1..41: mol_type = other DNA
gaaaattggttatgcagttgctgttagcaccaaaccggttg

SEQ ID NO: 305 - DNA, 41 bp, synthetic construct
  misc_feature 1..41: note = Synthetic
  source 1..41: mol_type = other DNA
caaccggtttggtgctaacatcaactgcataaccaattttc

SEQ ID NO: 306 - DNA, 41 bp, synthetic construct
  misc_feature 1..41: note = Synthetic
  source 1..41: mol_type = other DNA
gaaaattggttatgcagttgatgttagcaccaaaccggttg

SEQ ID NO: 307 - DNA, 41 bp, synthetic construct
  misc_feature 1..41: note = Synthetic
  source 1..41: mol_type = other DNA
tttggtgctaacaataactgcataaccaattttctggctcg

SEQ ID NO: 308 - DNA, 41 bp, synthetic construct
  misc_feature 1..41: note = Synthetic
  source 1..41: mol_type = other DNA
cgagccagaaaattggttatgcagttattgttagcaccaaa

SEQ ID NO: 309 - DNA, 47 bp, synthetic construct
  misc_feature 1..47: note = Synthetic
  source 1..47: mol_type = other DNA
aaccaaccggtttggtgctaacctgaactgcataaccaattttctgg

SEQ ID NO: 310 - DNA, 47 bp, synthetic construct
  misc_feature 1..47: note = Synthetic
  source 1..47: mol_type = other DNA
ccagaaaattggttatgcagttcaggttagcaccaaaccggttggtt

SEQ ID NO: 311 - DNA, 35 bp, synthetic construct
  misc_feature 1..35: note = Synthetic
  source 1..35: mol_type = other DNA
caaccggtttggtgctagcaacaactgcataacca

SEQ ID NO: 312 - DNA, 35 bp, synthetic construct
  misc_feature 1..35: note = Synthetic
  source 1..35: mol_type = other DNA
tggttatgcagttgttgctagcaccaaaccggttg

SEQ ID NO: 313 - DNA, 35 bp, synthetic construct
  misc_feature 1..35: note = Synthetic
  source 1..35: mol_type = other DNA
caaccggtttggtgctatcaacaactgcataacca

SEQ ID NO: 314 - DNA, 35 bp, synthetic construct
  misc_feature 1..35: note = Synthetic
  source 1..35: mol_type = other DNA
tggttatgcagttgttgatagcaccaaaccggttg

SEQ ID NO: 315 - DNA, 39 bp, synthetic construct
  misc_feature 1..39: note = Synthetic
  source 1..39: mol_type = other DNA
ccaaccggtttggtgctaataacaactgcataaccaatt

SEQ ID NO: 316 - DNA, 39 bp, synthetic construct
  misc_feature 1..39: note = Synthetic
  source 1..39: mol_type = other DNA
aattggttatgcagttgttattagcaccaaaccggttgg

SEQ ID NO: 317 - DNA, 47 bp, synthetic construct
  misc_feature 1..47: note = Synthetic
  source 1..47: mol_type = other DNA
aaccggtttggtgctctgaacaactgcataaccaattttctggctcg

SEQ ID NO: 318 - DNA, 47 bp, synthetic construct
  misc_feature 1..47: note = Synthetic
  source 1..47: mol_type = other DNA
cgagccagaaaattggttatgcagttgttcagagcaccaaaccggtt

(19) United States
(12) Patent Application Publication
(10) Pub. No.: US 2024/0327368 A1
(43) Pub. Date: Oct. 3, 2024

(54) BIOSYNTHETIC PRODUCTION OF GAMMA- OR DELTA-LACTONES USING CYTOCHROME P450 HYDROXYLASE ENZYMES OR MUTANTS THEREOF

(71) Applicant: Conagen Inc., Bedford, MA (US)

(72) Inventors: Haiyun Pan, Bedford, MA (US); Hui Chen, North Billerica, MA (US); Oliver Yu, Lexington, MA (US); Xiaoran Fu, Lincoln, MA (US)

(73) Assignee: Conagen Inc., Bedford, MA (US)

(21) Appl. No.: 18/492,054

(22) Filed: Oct. 23, 2023

Related U.S. Application Data
(63) Continuation of application No. PCT/US2022/025550, filed on Apr. 20, 2022.
(60) Provisional application No. 63/237,520, filed on Aug. 26, 2021.
(60) Provisional application No. 63/177,426, filed on Apr. 21, 2021.

(51) Int. Cl. C07D 309/30; C07D 307/32; C12N 9/02; C12R 1/19

(52) U.S. Cl. CPC C07D 309/30; C07D 307/32; C12N 9/0071; C12R 2001/19

(57) ABSTRACT

The present disclosure relates, at least in part, to the production of delta-lactones and gamma-lactones from fatty acid substrates (e.g., fatty acid substrates of C16-C25) via a batch fermentation method in an engineered microbe using a recombinant cytochrome P450 monooxygenase.

### RELATED APPLICATIONS

This application claims the benefit under 35 U.S.C. § 119(e) to U.S. Provisional Application No. 63/177,426 filed on Apr. 21, 2021 and entitled “BIOSYNTHETIC PRODUCTION OF DELTA-LACTONES USING CYTOCHROME P450 HYDROXYLASE MUTANT ENZYMES” and to U.S. Provisional Application No. 62/237,520 filed on Aug. 26, 2021 and entitled “BIOSYNTHETIC PRODUCTION OF GAMMA- OR DELTA-LACTONES USING CYTOCHROME P450 HYDROXYLASE ENZYMES OR MUTANTS THEREOF,” the entire contents of each of which are incorporated herein by reference.

### REFERENCE TO A SEQUENCE LISTING SUBMITTED AS A TEXT FILE

The instant application contains a Sequence Listing which has been submitted in ASCII format and is hereby incorporated by reference in its entirety. Said ASCII copy, created on Apr. 20, 2022, is named C149770079WO00-SEQ-ZJG and is 142,813 bytes in size.

### FIELD OF THE INVENTION

The present disclosure relates to methods and processes useful in the production of flavor- and fragrance-bearing compounds and specifically in the production of delta-lactone compounds via a one-step batch fermentation process. More specifically, the present disclosure provides for mutant enzymes that are regioselective to perform δ-hydroxylation on fatty acids, and the use of such mutant enzymes to convert fatty acids to delta-lactones. The present disclosure also relates to methods and processes useful in the production of flavor- and fragrance-bearing compounds and specifically in the production of gamma-lactone compounds via a one-step batch fermentation process. More specifically, the present disclosure provides for mutant enzymes that are regioselective to perform δ-hydroxylation on fatty acids, and the use of such mutant enzymes to convert fatty acids to gamma-lactones.

### BACKGROUND OF THE INVENTION

Interest in the production of flavor and fragrance compounds is widespread. The use of these compounds in food, detergents, cosmetics and pharmaceuticals is global. The world market was estimated to be close to $24 billion in 2013 (www.leffingwell.com), so the economic importance of these compounds is significant. The concepts of flavor and fragrance are complex and involves most of our senses (Barham P. et al., Molecular gastronomy: a new emerging scientific discipline, (2010) CHEM REV 110: 2313-65). However, the central component most often discussed for flavor is, of course, taste, which is sensed by receptors on the tongue. The human tongue is capable of distinguishing salt, sweet, bitter, sour, and umami. Smells are detected by sometimes amazingly sensitive receptors in the olfactory system in the nose. Many of these same receptors are in play for sensing fragrance. The chemical diversity in flavor and fragrance compositions is quite large, but in order to generate a smell or a taste, the compound must be sufficiently volatile that it can reach the sensory system in the upper part of the nose (Buck L., and Axel R., A novel multigene family may encode odorant receptors: a molecular basis for odor recognition (1991) CELL 65: 175-87; Lundström J. N. et al., Central processing of the chemical senses: an overview, (2011) ACS CHEM NEUROSCI 2:5). What has been reported includes sensory-directed identification of creaminess-enhancing volatiles and semivolatiles in full-fat cream (Schlutt et al., J. Agric. Food Chem. 2007, 55, 23, 9634-9645). What has been reported also includes a fat and oil enhancer that can be added to food and drink to enhance the fat and oil feeling and reduce the amount of fat and oil used (Japanese Patent Application Publication No. JP 2011083264 A). Chemical synthesis and extraction processes from plants and plant cells are the most common procedures for producing compounds that are important for flavor and fragrance compositions.

Plant extraction-based production has significant disadvantages, such as weather effects on the strength and abundance of the compounds of interest, risk of plant diseases and/or poor harvest, stability of the compound, environmental impact of increased production and trade restrictions. A longstanding alternative route is provided by chemical synthesis. Artificial synthetic processes suffer from few of the limitations present in plant-based extraction but yield compounds that, according to EU regulation (EC 1334/2008), are necessarily termed “flavoring substances” but are not viewed as “nature-identical” compounds, as prescribed in EC Directive 88/388. Since consumers are more and more strongly favoring ‘natural’ compounds, the price levels are substantially higher for those compounds that can be termed to be “nature-identical” (Schrader J. 2007. “Microbial flavour production” in FLAVOURS AND FRAGRANCES, Berger R G (ed.). Springer-Verlag: Berlin; 507-74) and the market has disfavored chemical synthesis-based approaches.

Lactones are important constituents contributing to aromas of various foods, such as fruits and dairy products. They occur, in low quantities, in fruits, like peach and coconut, but they bring about an important contribution to the typical taste of these products and confer their natural taste (An, J. U. and Oh, D. K. Increased production of γ-lactones from hydroxy fatty acids by whole Waltomyces lipofer cells induced with oleic acid. Appl Microbiol Biotechnol 97, 8265-8272 (2013)). A number of lactones exhibit antimicrobial, anticancer, and antiviral activities (Yang E. J., Kim Y. S. and Chang H. C. Purification and Characterization of Antifungal δ-Dodecalactone from Lactobacillus plantarum AF1 Isolated from Kimchi, Journal of Food Protection. 651-657 (2011)).

Odd- and even-numbered hydroxylated fatty acids are metabolized in the β-oxidation cycles of yeast and other fungi to 5-hydroxy and 4-hydroxy fatty acids, respectively, which may be further converted to delta-lactones and gamma-lactones (An & Oh, 2013). Example fatty acid substrates are illustrated in FIG. 5. Product delta-Lactones include δ-dodecalactone, δ-decalactone, δ-nonalactone, δ-undecalactone, etc., and gamma-lactones include γ-dodecalactone, γ-decalactone, γ-nonalactone, etc. (An & Oh, 2013). γ-Dodecalactone and δ-dodecalactone are two different types of lactones and can be used as precursors to different medicinal and flavoring compounds. Most lactones have been obtained directly from fruits or by chemical methods, but the increased demand for natural flavors in the marketplace has encouraged the development of processes that lead to natural flavoring substances.

Enzyme bioconversion is a good way for producing natural flavoring substances by converting a natural substrate into the desired materials. Many microbial processes have been described in the prior art that are able to produce interesting flavors, fragrances and aromas using lactone compounds. The primary issues in such production are that the compounds of interest are produced in extremely small amounts, cannot be produced reliably over time and can only be produced at high cost and/or require expensive procedures to acquire from naturally existing sources. That is, the compounds of interest are often present only in yields that are generally lower than needed to allow commercial success and exploitation. Therefore, the development of enhanced specific fermentation techniques and recovery methods may allow fragrances of interest to have much wider application in the food, fragrance and beverage industry while acting to provide cheaper prices for the general consumer as and when needed.

Unfortunately, traditional beta-oxidation processes suffered from low conversion yields that are believed to stem from the barrier effect of the cell wall or membrane. Cell permeabilization is believed to improve the transfer of the reaction substrate and product across the cell membrane and thus increases the production of metabolites, but reported titers available from traditional biosynthetic technologies are still low.

It is known that certain fungi can make various gamma-lactones de novo or upon the feeding of regular carboxylic acids without the involvement of beta-oxidation. This is believed to occur because the fungi have a built-in fatty acid 4-hydroxylase. For example, PCT International Publication No. WO 2020/018729 to Chen et al. discloses that a cytochrome P450 monooxygenase (GenBank No: GAN03094.1) from Mucor ambiguus can function on lauric acid as substrate to produce γ-dodecalactone. However, making delta-lactones from fatty acids via enzyme bioconversion at cost-effective, commercially viable rates and yields are not known.

Accordingly, a need exists for the development of a novel method of producing a delta-lactone economically and conveniently to further enable human and animal consumption.

### SUMMARY OF THE INVENTION

In one aspect, the present disclosure is focused on the conversion of a carboxylic acid into its corresponding delta-lactone (also referred to herein as “δ-lactone” or “delta lactone”), e. g., lauric acid to δ-dodecalactone by novel biosynthetic pathways, for instance via a microbial host expressing novel fatty acid 5-hydroxylase enzymes. In a representative embodiment, the present disclosure relates to the enzymatic conversion of lauric acid into δ-dodecalactone in recombinant bacteria.

It was previously shown that a cytochrome MaP450 monooxygenase (GenBank: GAN03094.1) from Mucor ambiguous can function on lauric acid substrate and produce γ-Dodecalactone (e.g., in WO2020/018727, incorporated herein by reference). The cytochrome MaP450 monooxygenase is also referred to herein as “cytochrome MaP450 hydroxylase.” The present disclosure, in some aspects, relate to variants of a cytochrome MaP450 monooxygenase that can efficiently convert fatty acids (e.g., lauric acid) to delta lactones (e.g., δ-Dodecalactone). In some embodiments, the variant comprises one or more amino acid substations at positions N86, S272 and S341 of SEQ ID NO: 1.

In some aspects of the present disclosure, a method (e.g., bioconversion method) for the production of a delta-lactone is provided, comprising growing a cellular system in a culture medium, wherein the cellular system comprises a host cell which has been modified to express a recombinant cytochrome P450 hydroxylase polypeptide comprising one or more amino acid substitutions at positions N86, S272 and S341 in SEQ ID NO: 1; expressing the recombinant cytochrome P450 hydroxylase polypeptide in the cellular system; exposing the cellular system to a substrate and NADPH, wherein said substrate is a carboxylic acid comprising a linear or branched, alkyl, alkenyl, or alkynyl moiety comprising five to thirty-four carbon atoms, a salt thereof, an alkyl ester thereof, a mono, di or triglyceride thereof or an unsubstituted monoalkyl or dialkyl amide thereof, thereby producing the delta-lactone in a recoverable amount.

In certain embodiments, the recoverable amount is at least 1 mg. In certain embodiments, the recoverable amount is at least 10 mg. In certain embodiments, the recoverable amount is between 1 mg and 100 mg, between 100 mg and 10 g, or between 10 g and 1 kg, inclusive. Some aspects of the present disclosure provide methods for the production of a delta-lactone, the method comprising:
growing a cellular system in a culture medium, wherein the cellular system comprises a host cell which has been modified to express a recombinant cytochrome P450 hydroxylase polypeptide comprising one or more amino acid substitutions at positions N86, S272 and S341 in SEQ ID NO: 1;
expressing the recombinant cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof, thereby producing the delta-lactone in a recoverable amount.

In some embodiments, the hydroxylase polypeptide converts a carboxylic acid substrate into a delta-hydroxy fatty acid. In some embodiments, the method further comprises acidifying the culture medium to convert the delta-hydroxylated fatty acid to a delta-lactone.

Some aspects of the present disclosure provide methods for the production of a delta-lactone, the method comprising:
incubating a cytochrome P450 polypeptide comprising one or more amino acid substitutions at positions N86, S272 and S341 in SEQ ID NO: 1, with a substrate that is a carboxylic acid and NADPH for a sufficient time to convert the substrate to a hydroxylated fatty acid composition comprising one or more hydroxylated fatty acids, wherein a delta-hydroxylated fatty acid is present at a ratio of at least 20% of all hydroxylated fatty acids present in the hydroxylated fatty acid composition; and
acidifying the hydroxylated fatty acid composition to convert the delta-hydroxylated fatty acid to a delta-lactone.

In some embodiments, the substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof.

In some embodiments, the fatty acid substrate is represented by Formula (I):

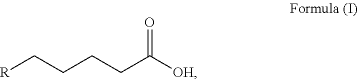

wherein R is a C₁₁₋₂₀ alkyl group, a C₁₁₋₂₀ alkenyl, or a C₁₁₋₂₀ alkynyl group.

In some embodiments, the delta-hydroxylated fatty acid is represented by Formula (II):

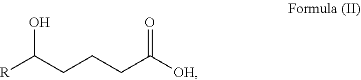

and the delta-lactone is represented by Formula (III):

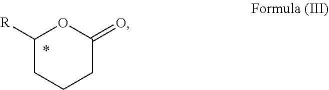

wherein R is a C₁₁₋₂₀ alkyl group, a C₁₁₋₂₀ alkenyl group, or a C₁₁₋₂₀ alkynyl group, and wherein * indicates a chiral carbon.

In some embodiments, R does not comprise a double bond. In some embodiments, R comprises one, two, three, or four double bonds. In some embodiments, each double bond is a Z double bond. In some embodiments, the delta-lactones do not comprise C═C═C. In some embodiments, the delta-lactones do not comprise C≡C.

In some embodiments, the substrate is a carboxylic acid comprising a linear alkyl, alkenyl, or alkynyl moiety comprising twenty to twenty-two carbon atoms. In some embodiments, the delta-lactone is of the formula:

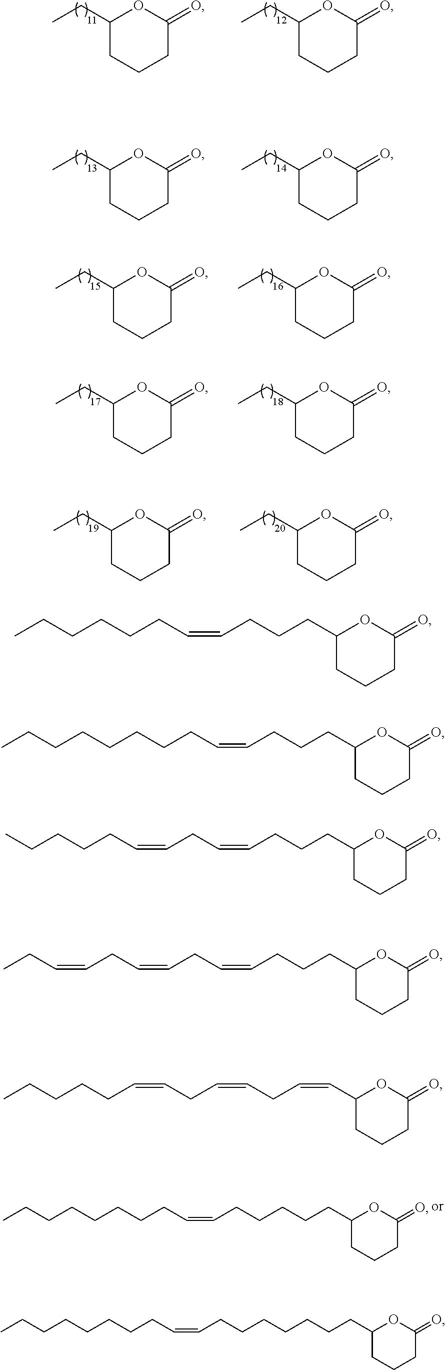

or a tautomer, isotopically labeled compound, solvate, polymorph, or co-crystal thereof; or a mixture thereof. In some embodiments, the chiral carbon atom is of the S configuration. In some embodiments, the chiral carbon atom is of the R configuration.
In some embodiments, the recombinant cytochrome P450 hydroxylase polypeptide comprises an amino acid substitution selected from S272I, S272L, S272M, S272N, S272T and N276T. In some embodiments, the recombinant cytochrome P450 hydroxylase polypeptide comprises amino acid substitutions selected from S272N/N86E, S272N/N86M, S272N/S341G, S272N/S341H, S272N/S341N, S272T/N86F, S272T/N86I, S272T/N86V. In some embodiments, the recombinant cytochrome P450 hydroxylase polypeptide comprises amino acid substitutions selected from S272N/N86M/S341D, S272N/N86M/S341H, S272T/N86F/S341A, S272T/N86F/S341C, S272T/N86F/S341H, S272T/N86F/S341M, S272T/N86F/S341Q. In some embodiments, the recombinant cytochrome P450 hydroxylase polypeptide comprises an amino acid sequence at least 70% (e.g., at least 70%, at least 75%, at least 80%, at least 85%, at least 90%, at least 95%, at least 98%, or at least 99%) identical to that of SEQ ID NOs: 3, 5, 7, 9, or 11. In some embodiments, the recombinant cytochrome P450 hydroxylase polypeptide comprises the amino acid sequence of any one of SEQ ID NOs: 3, 5, 7, 9, or 11.

In some embodiments, said host cell is a bacterium, a yeast cell, a fungal cell, an alga cell, or a plant cell. In some embodiments, the host cell is bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium. In some embodiments, the host cell is a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys. In some embodiments, the host cell is E. coli.

In some embodiments, the delta-lactone has a purity of not less than 50% (e.g., not less than 50%, not less than 60%, not less than 70%, not less than 80%, not less than 90%, or not less than 99%). In some embodiments, the delta-lactone has a purity of not less than 75% (e.g., not less than 75%, not less than 85%, not less than 95%).

Further provided herein are delta-lactones, or a tautomer, isotopically labeled compound, solvate, polymorph, or co-crystal thereof, produced by the method described herein. Mixtures of two or more lactones are also provided, wherein each lactone is independently a delta-lactone produced by the method described herein, or tautomer, isotopically labeled compound, solvate, polymorph, or co-crystal thereof. Further provided herein are compositions comprising the delta-lactone or a tautomer, isotopically labeled compound, solvate, polymorph, or co-crystal thereof. In some embodiments, the composition further comprises a pharmaceutically acceptable excipient, cosmetically acceptable excipient, or nutraceutically acceptable excipient.

Other aspects of the present disclosure provide methods for the production of a gamma-lactone, the method comprising:
growing a cellular system in a culture medium, wherein the cellular system comprises a host cell which has been modified to express a recombinant cytochrome P450 hydroxylase polypeptide comprising an amino acid sequence that is at least 80% identical to SEQ ID NO: 1;
expressing the recombinant cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof, thereby producing the gamma-lactone in a recoverable amount. In some embodiments, the carboxylic acid comprising a linear or branched, alkyl, alkenyl, or alkynyl moiety comprising twenty-one to twenty-five carbon atoms.

In some embodiments, the hydroxylase polypeptide converts a carboxylic acid substrate into a gamma-hydroxy fatty acid. In some embodiments, the method further comprises acidifying the culture medium to convert the gamma-hydroxylated fatty acid to a gamma-lactone.

Further provided herein are methods for the production of a gamma-lactone, the method comprising:
incubating a cytochrome P450 polypeptide comprising an amino acid sequence that is at least 80% identical to SEQ ID NO: 1, with a substrate that is a carboxylic acid and NADPH for a sufficient time to convert the substrate to a hydroxylated fatty acid composition comprising one or more hydroxylated fatty acids, wherein a gamma-hydroxylated fatty acid is present at a ratio of at least 20% of all hydroxylated fatty acids present in the hydroxylated fatty acid composition; and
acidifying the hydroxylated fatty acid composition to convert the gamma-hydroxylated fatty acid to a gamma-lactone.

In some embodiments, the substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof. In some embodiments, the carboxylic acid comprising a linear or branched, alkyl, alkenyl, or alkynyl moiety comprising twenty-one to twenty-five carbon atoms.

In some embodiments, the gamma-lactone is represented by Formula (IV):

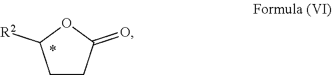

wherein R² is a C₁₂₋₂₁ alkyl group, a C₁₂₋₂₁ alkenyl group, or a C₁₂₋₂₁ alkynyl group, and wherein * indicates a chiral carbon.

In some embodiments, R² is does not comprise a double bond. In some embodiments, R² comprises one, two, three, or four double bonds. In some embodiments, each double bond is a Z double bond. In some embodiments, the gamma-lactones do not comprise C═C═C. In some embodiments, the gamma-lactones do not comprise C≡C. In some embodiments, the substrate is a carboxylic acid comprising a linear alkyl, alkenyl, or alkynyl moiety comprising twenty to twenty-two carbon atoms.

In some embodiments, the gamma-lactone is of the formula:

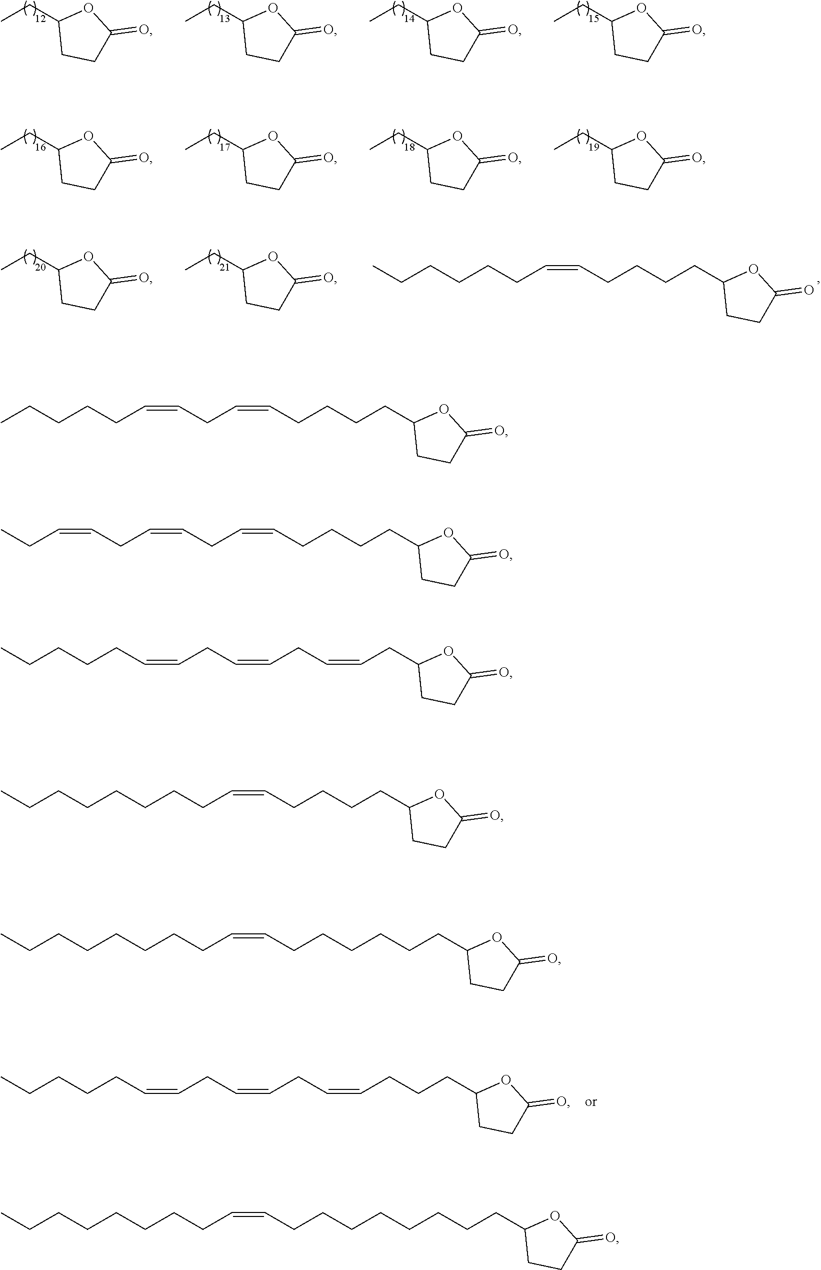

or a tautomer, isotopically labeled compound, solvate, polymorph, or co-crystal thereof; or a mixture thereof. In some embodiments, the chiral carbon atom is of the S configuration. In some embodiments, the chiral carbon atom is of the R configuration.

In some embodiments, the recombinant cytochrome P450 hydroxylase polypeptide comprises an amino acid sequence at least 90% identical to that of SEQ ID NO: 1. In some embodiments, wherein the recombinant cytochrome P450 hydroxylase polypeptide comprises the amino acid sequence of SEQ ID NO: 1.

In some embodiments, said host cell is a bacterium, a yeast cell, a fungal cell, an alga cell, or a plant cell. In some embodiments, the host cell is bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium. In some embodiments, the host cell is a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys. In some embodiments, the host cell is E. coli.

In some embodiments, the gamma-lactone has a purity of not less than 50% (e.g., not less than 50%, not less than 60%, not less than 70%, not less than 80%, not less than 90%, or not less than 99%). In some embodiments, the gamma-lactone has a purity of not less than 75% (e.g., not less than 75%, not less than 85%, not less than 95%).

Further provided herein are gamma-lactones, or a tautomer, isotopically labeled compound, solvate, polymorph, or co-crystal thereof, produced by the method described herein. Mixtures of two or more lactones are also provided, wherein each lactone is independently a delta-lactone produced by the method described herein, or tautomer, isotopically labeled compound, solvate, polymorph, or co-crystal thereof. Further provided herein are compositions comprising the gamma-lactone or a tautomer, isotopically labeled compound, solvate, polymorph, or co-crystal thereof. In some embodiments, the composition further comprises a pharmaceutically acceptable excipient, cosmetically acceptable excipient, or nutraceutically acceptable excipient.

Further provided herein are delta lactones represented by the formula:

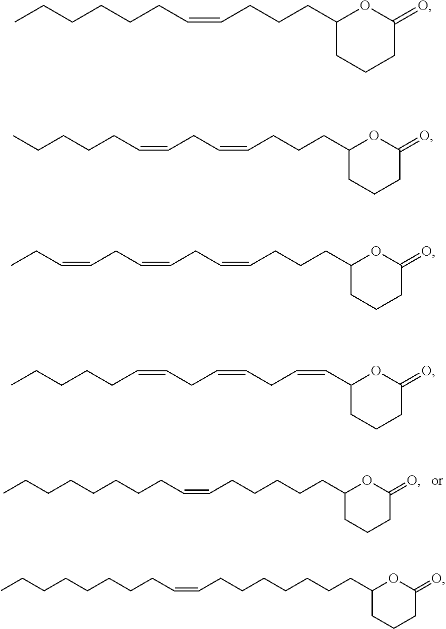 [SMILES: CCCCCC/C=C\CCCC1CCCC(=O)O1 ; CCCCC/C=C\C/C=C\CCCC1CCCC(=O)O1 ; CC/C=C\C/C=C\C/C=C\CCCC1CCCC(=O)O1 ; CCCCC/C=C\C/C=C\C/C=C\C1CCCC(=O)O1 ; CCCCCCCC/C=C\CCCCCC1CCCC(=O)O1 ; CCCCCCCC/C=C\CCCCCCCC1CCCC(=O)O1]

or a tautomer, isotopically labeled compound, solvate, polymorph, or co-crystal thereof.

Further provided herein are gamma lactones represented by the formula:

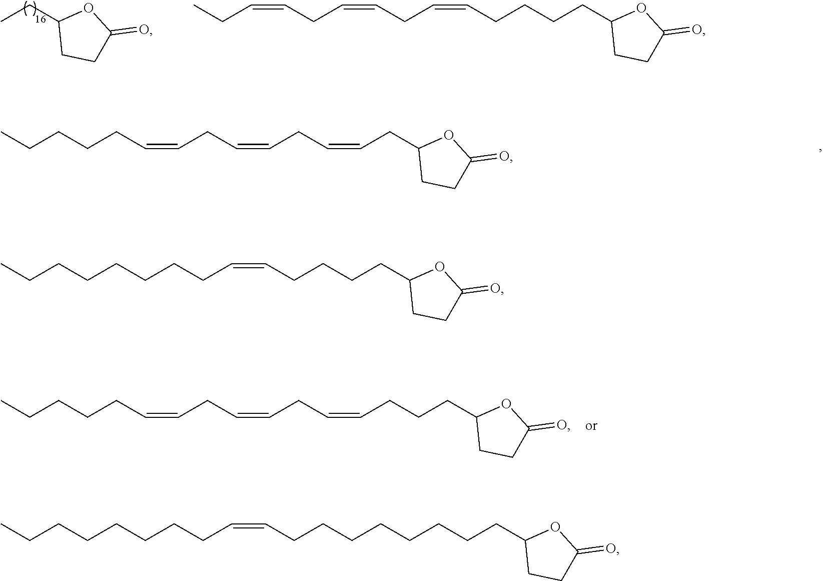

or a tautomer, isotopically labeled compound, solvate, polymorph, or co-crystal thereof.

Composition comprising any one of the delta lactones and/or any one of the gamma lactones provided herein are provided. In some embodiments, the composition further comprises a pharmaceutically acceptable excipient, cosmetically acceptable excipient, or nutraceutically acceptable excipient.

### BRIEF DESCRIPTION OF THE DRAWINGS

FIG. 1 illustrates a docking study where a lauric acid molecule was docked into a modeling structure of a MaP450 enzyme from Mucor ambiguus.

FIG. 2 compares GC/MS chromatograms of the hydroxylation products obtained with a wild-type MaP450 enzyme from Mucor ambiguus and its mutant S272N.

FIG. 3 illustrates biosynthetic steps in the production of γ- and δ-dodecalactone from lauric acid.

FIG. 4A illustrates example fatty acid precursors. FIG. 4B illustrates the cyclization of fatty acid precursors into corresponding delta-lactones formed by the action of a novel fatty acid C5-hydroxylase.

FIG. 5 includes the structures of exemplary carboxylic acids used as substrates for delta-lactone production.

FIG. 6 Residues targeted are depicted in MaP450 triple mutant S272T/N86F/S341H with bound lauric acid FIG. 7 shows GC-MS analysis of wild type and triple mutant S272T/N86F/S341H enzyme activities.

FIG. 8 shows additional exemplary fatty acids.

FIG. 9 shows a GC/MS analysis of gamma-lactones derived from different fatty acids.

The identity of each gamma-lactone was confirmed by its molecular weight and retention time when a standard was available. The initial concentration of fatty acid was 1 g/L in the bioconversion mixture. Samples were taken 5 h after the reaction. GC/MS Method 1 was used for the analysis.

FIG. 10 shows a GC/MS analysis of delta-lactones derived from different fatty acids. The identity of each delta-lactone was confirmed by its molecular weight and retention time when a standard was available. The initial concentration of fatty acid was 2 g/L in the bioconversion mixture. Samples were taken 5 h after the reaction. GC/MS Method 2 was used for the analysis of DC12 to DC16, and GC/MS Method 1 was used for the analysis of DC17 to DC22:1.

### Definitions

When a range of values (“range”) is listed, it is intended to encompass each value and subrange within the range. A range is inclusive of the values at the two ends of the range unless otherwise provided. For example, “C₇₋₁₃ alkyl” encompasses, e.g., C₇ alkyl, C₁₃ alkyl, and C₈₋₁₀ alkyl.

The term “alkyl” refers to a radical of a branched or unbranched, saturated acyclic hydrocarbon group. In certain embodiments, the alkyl has between 4 and 30 carbon atoms “C₄₋₃₀ alkyl.”

The term “alkenyl” refers to a radical of a branched or unbranched, acyclic hydrocarbon group having one or more carbon-carbon double bonds (C═C bonds; e.g., 1, 2, 3, 4, 5, or 6 C═C bonds), as valency permits. In alkenyl groups,

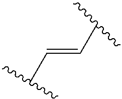

is an E double bond, and

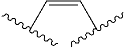 [SMILES: */C=C\*]

is an Z double bond. Other situations involving an E or Z double bond are as known in the art. In an alkenyl group, a C═C bond for which the stereochemistry is not specified (e.g., —CH═CH— or

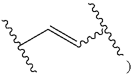

may be a E or Z double bond. In certain embodiments, the alkenyl has between 4 and 30 carbon atoms (“C₄₋₃₀ alkenyl”). Alkenyl may further include one or more carbon-carbon triple bonds (C≡C bonds).

The term “alkynyl” refers to a radical of a branched or unbranched, acyclic hydrocarbon group having one or more carbon-carbon triple bonds (e.g., 1, 2, 3, or 4 carbon-carbon triple bonds), as valency permits. In certain embodiments, the alkynyl has between 4 and 30 carbon atoms (“C₄₋₃₀ alkynyl”). Alkynyl may further include one or more C═C bonds.

Affixing the suffix “ene” to a group indicates the group is a divalent moiety, e.g., alkylene is a divalent moiety of alkyl, alkenylene is a divalent moiety of alkenyl, and alkynylene is a divalent moiety of alkynyl.

A “lactone” is a monocyclic compound where the moiety —C(═O)—O— is part of the monocyclic ring, and the remaining part of the monocyclic compound is alkylene, alkenylene, or alkynylene. When the alkylene, alkenylene, or alkynylene is branched, the lactone also includes the branch(es) of the alkylene, alkenylene, or alkynylene. A delta-lactone is a compound of the formula:

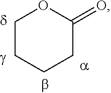

or a tautomer, isotopically labeled compound, solvate, polymorph, or co-crystal thereof, wherein the carbon atoms at the α, β, γ, and δ positions may be independently substituted or unsubstituted. A “γ-lactone,” “gamma-lactone,” or “gamma lactone” is a compound of the formula:

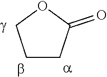

or a tautomer, isotopically labeled compound, solvate, polymorph, or co-crystal thereof, wherein the carbon atoms at the α, β, and γ positions may be independently substituted or unsubstituted.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol tautomerization.

Compounds (e.g., lactones) that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3-, 10-, 30-, 100-, 300-, 1,000-, 3,000- or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C or ¹⁸O.

The term “salt” refers to ionic compounds that result from the neutralization reaction of an acid (e.g., a fatty acid) and a base. A salt is composed of one or more cations (positively charged ions) and one or more anions (negative ions) so that the salt is electrically neutral (without a net charge). The salt may be an alkali metal salt, alkaline earth metal salt, ammonium salt, and N⁺(C₁₋₄ alkyl)₄ salt. Alkali metals and alkaline earth metals include, for example, sodium, potassium, lithium, calcium, and magnesium.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates and ethanolates.

The term “polymorph” refers to a crystalline form of a compound (or a solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a provided compound and another substance), wherein each of the components is independently an atom, ion, or molecule. In certain embodiments, none of the components is a solvent. In certain embodiments, at least one of the components is a solvent. A co-crystal of a provided compound and another substance is different from a salt formed from a provided compound and another substance. In the salt, a provided compound is complexed with another substance in a way that proton transfer (e.g., a complete proton transfer) between another substance and the provided compound easily occurs at room temperature. In the co-crystal, however, a provided compound is complexed with another substance in a way that proton transfer between another substance to the provided herein does not easily occur at room temperature. In certain embodiments, in the co-crystal, there is substantially no proton transfer from another substance to a provided compound. In certain embodiments, in the co-crystal, there is partial proton transfer from another substance to a provided compound. Co-crystals may be useful to improve the properties (e.g., solubility, stability, and ease of formulation) of a provided compound.

“Cellular system” are any cells that provide for the expression of ectopic proteins. It includes bacteria, yeast, plant cells and animal cells. It may include prokaryotic or eukaryotic host cells which are modified to express a recombinant protein and cultivated in an appropriate culture medium. It also includes the in vitro expression of proteins based on cellular components, such as ribosomes.

“Coding sequence” is to be given its ordinary and customary meaning to a person of ordinary skill in the art, and is used without limitation to refer to a DNA sequence that encodes for a specific amino acid sequence.

“Growing the Cellular System”. Growing includes providing an appropriate medium that would allow cells to multiply and divide, to form a cell culture. It also includes providing resources so that cells or cellular components can translate and make recombinant proteins.

“Protein Expression”. Protein production can occur after gene expression. It consists of the stages after DNA has been transcribed to messenger RNA (mRNA). The mRNA is then translated into polypeptide chains, which are ultimately folded into proteins. DNA or RNA may be present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

“Yeast”. According to the current disclosure a yeast are eukaryotic, single-celled microorganisms classified as members of the fungus kingdom. Yeasts are unicellular organisms which are believed to have evolved from multicellular ancestors.

As used herein, the singular forms “a, an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subject technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. Unless otherwise indicated, a particular nucleic acid sequence also implicitly encompasses conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing a delta- or gamma-lactone composition.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,” and “peptide” are to be given their respective ordinary and customary meanings to a person of ordinary skill in the art; the three terms are sometimes used interchangeably, and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides, and proteins, unless otherwise noted. The terms “protein,” “polypeptide,” and “peptide” are used interchangeably herein when referring to a polyaminoacid product. Thus, exemplary polypeptides include polyaminoacid products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another; the substitution of one charged or polar (hydrophilic) residue for another such as between arginine and lysine, between glutamine and asparagine, between threonine and serine; the substitution of one basic residue such as lysine or arginine for another; or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide.

The term “homologous” in all its grammatical forms and spelling variations refers to the relationship between polynucleotides or polypeptides that possess a “common evolutionary origin,” including polynucleotides or polypeptides from super-families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96%, at least 97%, at least 98%, at least 99%, and even 100% identical.

“Suitable regulatory sequences” is to be given its ordinary and customary meaning to a person of ordinary skill in the art, and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art, and is used without limitation to refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art, and is used without limitation to refer to the transcription and stable accumulation of sense (mRNA) or antisense RNA derived from the nucleic acid fragment of the subject technology. “Over-expression” refers to the production of a gene product in transgenic or recombinant organisms that exceeds levels of production in normal or non-transformed organisms.

“Transformation” is to be given its ordinary and customary meaning to a person of reasonable skill in the field, and is used without limitation to refer to the transfer of a polynucleotide into a target cell for further expression by that cell. The transferred polynucleotide can be incorporated into the genome or chromosomal DNA of a target cell, resulting in genetically stable inheritance, or it can replicate independent of the host chromosomal DNA. Host organisms containing the transformed nucleic acid fragments are referred to as “transgenic” or “recombinant” or “transformed” organisms.

The terms “transformed,” “transgenic,” and “recombinant,” when used herein in connection with host cells, are to be given their respective ordinary and customary meanings to a person of ordinary skill in the art, and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art, and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

### DETAILED DESCRIPTION

In one aspect, provided herein are methods that utilize fatty acids or their derivatives as substrates for recombinant cell systems and/or enzymes to produce delta-lactones. A solution to the problems associated with synthetic chemistry-based approaches is exemplified in the present disclosure, that is, through the use of genetically modified enzymes and cell cultures to prepare/convert or create the substances of interest. The methods, enzymes, and cell cultures of the present disclosure can do so in controlled environments with a smaller environmental footprint while consistently delivering compounds via fermentation processes that can be identified as “nature, identical” pursuant to EU regulations and free of the limitations of plant-based extraction or synthetic chemistry.

According to one embodiment, delta-lactones may be reliably produced at high yields by gene modification and fermentation technologies using cell systems, e.g., bacterial cultures. These microorganisms are able to synthesize delta-lactones de novo or by biotransformation of fatty acids to provide commercially significant yields. New production methods are provided to reduce costs of delta-lactone production and lessen the environmental impact of large-scale cultivation and processing (Yao et al., 1994) of natural sources for this type of molecule. The use of a cell culture-based approach to produce lactones has advantages over synthetic methods because a cell culture-based process typically combines into a single step the multiple reactions required by a synthetic method. Moreover, the biosynthetic process would satisfy the desire to obtain flavor, fragrance, and pharmaceutical materials from natural sources without the associated detrimental environmental impact.

In a first set of exemplary embodiments, the present disclosure relates to the biosynthetic production of a delta-lactone from a carboxylic acid substrate through the use of a novel, recombinant P450 hydroxylase enzyme. Hence, the recombinant polypeptide of the subject technology is useful for the biosynthesis of delta-lactone compounds. The substrate may be a linear or branched carboxylic acid comprising six to thirty-five carbon atoms (including the carbon atom of the carbonyl moiety). The substrate may be a linear or branched carboxylic acid comprising nine to thirty-five carbon atoms, including the carbon atom of the carbonyl moiety. The substrate may be a linear or branched carboxylic acid comprising five to fifteen carbon atoms. Typical substrates include fatty acids featuring alkyl moieties or alkenyl moieties bearing one, two, or three unsaturations. In certain embodiments, the fatty acid is naturally occurring. Also included are fatty acid derivatives, such as their salts, esters, mono, di, and triglycerides, monoalkyl and dialkyl amides. In an embodiment, a carboxylic (—C(O)O—) group of the substrate is covalently linked to a carbon atom of a linear alkyl or alkenyl chain featuring at least five carbon atoms to not less than fifteen carbon atoms. The substrates may be transformed into delta-lactones or, for instance, those lactone derivatives that are made through compound desaturation, branching, hydroxylation, esterification or saponification. The substrates may also be transformed into gamma-lactones.

Without being bound to any theory, it is believed that the carboxylic acids and the corresponding derivatives are hydoxylated at their C5 position by a recombinant cell, e.g., a modified microbial host expressing a recombinant P450 cyctochrome hydroxylase according to the present disclosure. The resulting 5-hydroxyacids are then cyclized, usually upon acidification, to form the corresponding delta-lactones or delta-lactones substituted with desired functional groups.

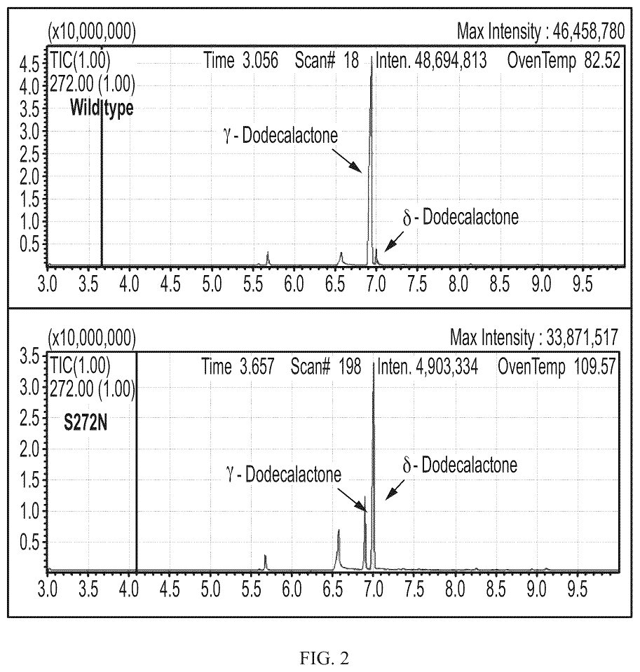

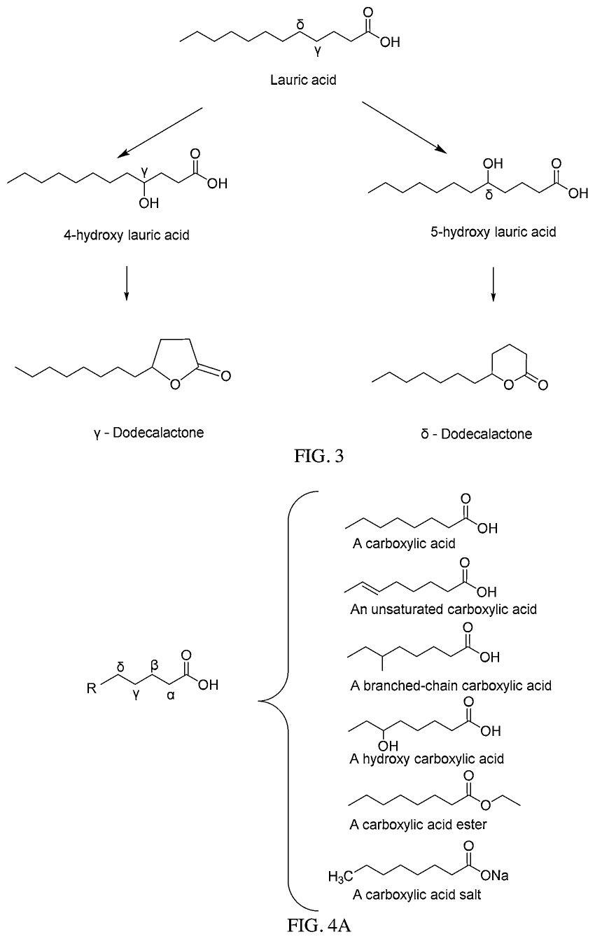

The aforementioned WO 2020/018729 discloses a wild type cytochrome P450 monooxygenase (GenBank: GAN03094.1) from Mucor ambiguus which can catalyze the C4 hydroxylation of lauric acid and produce γ-dodecalactone. Reported herein is the discovery that the wild type enzyme can also produce small amounts of δ-dodecalactone. Also reported herein is the discovery that the wild type enzyme can also catalyze the C5 hydroxylation of a fatty acid to produce the corresponding 5-hydroxy fatty acid, which can undergo lactonization (e.g., under acidic conditions) to produce delta-lactones. Specifically, when lauric acid was used as the substrate, gas chromatography-mass spectrometry (GC-MS) analysis of the products revealed the presence of δ-dodecalactone. As illustrated in FIG. 2, the relative amounts of γ-dodecalactone to δ-dodecalactone were produced at a ratio of 98.1:1.9. A comparison of the putative biosynthetic pathways leading to either γ- or δ-cyclization of the substrate lauric acid is provided in FIG. 3.

In addition, and also as illustrated in FIG. 2, reported herein are mutagenesis studies that produced a P450 mutant enzyme S272N (SEQ ID NO: 3) which is capable of increasing the yield in δ-dodecalactone by 21-fold as compared to the wild type enzyme, thereby changing the ratio of product γ-dodecalactone to δ-dodecalactone to 23.4:76.6. The mutagenesis studies identified residues that may be mutated to control fatty acid rotation, bend and motion and to produce different ratios of product gamma-lactone to delta-lactone, the residues including one or more of N86, S272, N276, and S341. In one representative embodiment, the recombinant P450 hydroxylase enzyme of SEQ ID NO: 3 and its variants allow for the biosynthesis of large amounts of delta-lactones of interest. In one specific embodiment, the present disclosure provides for the production of δ-dodecalactone from n-dodecanoic acid (also known as lauric acid) via an enzymatic conversion step catalyzed by the aforementioned recombinant P450 hydroxylase enzyme of SEQ ID NO: 3 or its variants.

In some embodiments, the cytochrome P450 hydroxylase polypeptide comprises one or more (e.g., 1, 2, or 3) amino acid substitutions at positions N86, S272 and S341 in SEQ ID NO: 1. In some embodiments, the cytochrome P450 hydroxylase polypeptide comprises an amino acid substitution selected from S272I, S272L, S272M, S272N, S272T, and N276T in SEQ ID NO: 1. In some embodiments, the cytochrome P450 hydroxylase polypeptide comprises an S272N substitution in SEQ ID NO: 1. In some embodiments, the cytochrome P450 hydroxylase polypeptide comprises an S272T substitution in SEQ ID NO 1.

In some embodiments, the cytochrome P450 hydroxylase polypeptide comprises two amino acid substitutions selected from S272N/N86E, S272N/N86M, S272N/S341G, S272N/S341H, S272N/S341N, S272T/N86F, S272T/N86I, and S272T/N86V in SEQ ID NO: 1. “/” indicates more than one mutation. In some embodiments, the cytochrome P450 hydroxylase polypeptide comprises three amino acid substitutions selected from S272N/N86M/S341D, S272N/N86M/S341H, S272T/N86F/S341A, S272T/N86F/S341C, S272T/N86F/S341H, S272T/N86F/S341M, and S272T/N86F/S341Q in SEQ ID NO: 1. In some embodiments, the cytochrome P450 hydroxylase polypeptide comprises S272N/N86M/S341D substitutions in SEQ ID NO: 1.

In some embodiments, the cytochrome P450 hydroxylase polypeptide comprises an amino acid sequence that is at least 70% (e.g., at least 70%, at least 75%, at least 80%, at least 85%, at least 90%, at least 95%, or at least 99%) identical to SEQ ID NO: 1 and comprises one or more (e.g., 1, 2, or 3) amino acid substitutions at positions N86, S272 and S341. In some embodiments, the cytochrome P450 hydroxylase polypeptide comprises an amino acid sequence that is at least 70% (e.g., at least 70%, at least 75%, at least 80%, at least 85%, at least 90%, at least 95%, or at least 99%) identical to SEQ ID NO: 1 and comprises an amino acid substitution selected from S272I, S272L, S272M, S272N, S272T, and N276T (e.g., S272N or S272T). In some embodiments, the cytochrome P450 hydroxylase polypeptide comprises an amino acid sequence that is at least 70% (e.g., at least 70%, at least 75%, at least 80%, at least 85%, at least 90%, at least 95%, or at least 99%) identical to SEQ ID NO: 1 and comprises two amino acid substitutions selected from S272N/N86E, S272N/N86M, S272N/S341G, S272N/S341H, S272N/S341N, S272T/N86F, S272T/N86I, and S272T/N86V. In some embodiments, the cytochrome P450 hydroxylase polypeptide comprises an amino acid sequence that is at least 70% (e.g., at least 70%, at least 75%, at least 80%, at least 85%, at least 90%, at least 95%, or at least 99%) identical to SEQ ID NO: 1 and comprises three amino acid substitutions selected from selected from S272N/N86M/S341D, S272N/N86M/S341H, S272T/N86F/S341A, S272T/N86F/S341C, S272T/N86F/S341H, S272T/N86F/S341M, and S272T/N86F/S341Q (e.g., S272N/N86M/S341D).

In some embodiments, the cytochrome P450 hydroxylase polypeptide comprises the amino acid sequence of any one of SEQ ID NOs: 3, 5, 7, 9, or 11.

The foregoing results are important in that different mutants capable of producing different types and different yields of delta-lactone products are of high industrial interest. The 5-hydroxylase activity of the recombinant enzyme may be used either in vivo or in vitro for the production of a number of delta-lactones from various carboxylic acid substrates. In related embodiments, the novel enzymes find use in heterologous systems for the production delta-lactones for use in a variety of industries and may be introduced into recombinant host organisms for commercial production of these compounds. In some embodiments, the delta-lactones are C16-C25 (e.g., C16-C25, C16-C20, C25-C25) delta-lactones. In some embodiments, the delta-lactones are C16, C17, C18, C19, C20, C21, C22, C23, C24, or C25 delta-lactones. In some embodiments, the delta-lactones are C20-C22 (e.g., C20, C21, or C22) delta-lactones. Representative product lactones include δ-nepetalactone; δ-octalactone; δ-nonalactone; δ-decalactone; δ-undecalactone; δ-dodecalactone; δ-tridecalactone, δ-tetradecalactone, and δ-pentadecalactone.

As demonstrated herein, in some embodiments, the wild-type P450 enzyme can be used to produce C16-C25 (e.g., C16-C25, C16-C20, C25-C25) gamma-lactones. In some embodiments, the gamma-lactones are C16-C25 (e.g., C16-C25, C16-C20, C25-C25) gamma-lactones. In some embodiments, the gamma-lactones are C16, C17, C18, C19, C20, C21, C22, C23, C24, or C25 gamma-lactones. In some embodiments, the gamma-lactones are C20-C22 (e.g., C20, C21, or C22) gamma-lactones.

In certain embodiments, the product delta-lactone is represented by Formula (V):

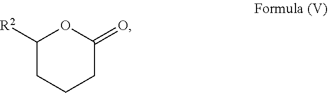

or a tautomer, isotopically labeled compound, solvate, polymorph, or co-crystal thereof, or a mixture thereof, wherein R² is unsubstituted, branched or unbranched, C₄₋₃₀ alkyl, C₄₋₃₀ alkenyl, or C₄₋₃₀ alkynyl. In some embodiments, R² is a hydrogen. In embodiments, R² is an unsubstituted, branched or unbranched, C₁₁₋₂₀ (e.g., C11, C12, C13, C14, C15, C16, C17, C18, C19, C20) alkyl, C₁₁₋₂₀ (e.g., C11, C12, C13, C14, C15, C16, C17, C18, C19, C20) alkenyl, or C₁₁₋₂₀ (e.g., C11, C12, C13, C14, C15, C16, C17, C18, C19, C20) alkynyl. In embodiments, R² is an unsubstituted unbranched, C₁₆₋₁₈ (e.g., C16, C17, or C18) alkyl, C₁₆₋₁₈ (e.g., C16, C17, or C18) alkenyl, or C₁₆₋₁₈ (e.g., C16, C17, or C18) alkynyl. When R² is C11-C20, the delta lactone is C16-C25. When R² is C16-C18, the delta lactone is C20-C22.

In embodiments, R² is an unsubstituted unbranched, C₁₁₋₂₀ (e.g., C11, C12, C13, C14, C15, C16, C17, C18, C19, C20) alkyl, C₁₁₋₂₀ (e.g., C11, C12, C13, C14, C15, C16, C17, C18, C19, C20) alkenyl, or C11-20 (e.g., C11, C12, C13, C14, C15, C16, C17, C18, C19, C20) alkynyl. In embodiments, R² is an unsubstituted, branched or unbranched, C₁₆₋₁₈ (e.g., C16, C17, or C18) alkyl, C₁₆₋₁₈ (e.g., C16, C17, or C18) alkenyl, or C₁₆₋₁₈ (e.g., C16, C17, or C18) alkynyl. When R² is C11-C20, the delta lactone is C16-C25. When R² is C16-C18, the delta lactone is C20-C22.

In certain embodiments, the product gamma-lactone is represented by Formula (VI):

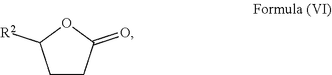

or a tautomer, isotopically labeled compound, solvate, polymorph, or co-crystal thereof, or a mixture thereof, wherein R² is unsubstituted, branched or unbranched, C₄₋₃₀ alkyl, C₄₋₃₀ alkenyl, or C₄₋₃₀ alkynyl. In some embodiments, wherein R² is unsubstituted, branched or unbranched, C₁₂₋₂₁ (e.g., C12, C13, C14, C15, C16, C17, C18, C19, C20, or C21) alkyl, C₁₂₋₂₁ (e.g., C12, C13, C14, C15, C16, C17, C18, C19, C20, or C21) alkenyl, or C₁₂₋₂₁ (e.g., C12, C13, C14, C15, C16, C17, C18, C19, C20, or C21) alkynyl. In some embodiments, wherein R² is unsubstituted, branched or unbranched, C₁₆₋₂₁ (e.g., C16, C17, C18, C19, C20, or C21) alkyl, C₁₆₋₂₁ (e.g., C16, C17, C18, C19, C20, or C21) alkenyl, or C₁₂₋₂₁ C₁₆₋₂₁ (e.g., C16, C17, C18, C19, C20, or C21) alkynyl. In some embodiments, wherein R² is unsubstituted, branched or unbranched, C₁₆₋₁₈ (e.g., C16, C17, C18) alkyl, C₁₆₋₁₈ (e.g., C16, C17, C18) alkenyl, or C₁₆₋₁₈ (e.g., C16, C17, C18) alkynyl. When R2 is C12-C21, the gamma lactone is C16-C25. When R2 is C16-C21, the gamma lactone is C20-C25. When R2 is C16-C18, the gamma lactone is C20-C22.

In certain embodiments, R² is unsubstituted, branched or unbranched, C₄₋₃₀ alkyl. In certain embodiments, R² is unsubstituted unbranched C₄₋₃₀ alkyl. In certain embodiments, R² is unsubstituted unbranched C₄₋₂₄ alkyl. In certain embodiments, R² is unsubstituted unbranched C₇₋₁₈ alkyl. In certain embodiments, R² is unsubstituted, branched or unbranched, C₄₋₃₀ alkenyl. In certain embodiments, R² is unsubstituted unbranched C₄₋₃₀ alkenyl. In certain embodiments, R² is unsubstituted unbranched C₆₋₂₄ alkenyl. In certain embodiments, R² is unsubstituted unbranched C₁₋₁₇ alkenyl. In certain embodiments, R² is unsubstituted, branched or unbranched, C₄₋₃₀ alkynyl. In certain embodiments, R² is unsubstituted unbranched C₄₋₃₀ alkynyl. In certain embodiments, R² is unsubstituted unbranched C₆₋₂₄ alkynyl. In certain embodiments, R² is unsubstituted unbranched C₁₋₁₇ alkynyl. In certain embodiments, R² is unsubstituted unbranched C₁₂₋₂₁ (e.g., C12, C13, C14, C15, C16, C17, C18, C19, C20, or C21) alkyl. In certain embodiments, R² is unsubstituted unbranched C₁₂₋₂₁ (e.g., C12, C13, C14, C15, C16, C17, C18, C19, C20, or C21) alkenyl. In certain embodiments, R² is unsubstituted unbranched C₁₂₋₂₁ (e.g., C12, C13, C14, C15, C16, C17, C18, C19, C20, or C21) alkynyl. In certain embodiments, R² is unsubstituted unbranched C₁₆₋₂₁ (e.g., C16, C17, C18, C19, C20, or C21) alkyl. In certain embodiments, R² is unsubstituted unbranched C₁₆₋₂₁ (e.g., C16, C17, C18, C19, C20, or C21) alkenyl. In certain embodiments, R² is unsubstituted unbranched C₁₆₋₂₁ (e.g., C16, C17, C18, C19, C20, or C21) alkynyl. In certain embodiments, R² is unsubstituted unbranched C₁₆₋₁₈ (e.g., C16, C17, C18) alkyl. In certain embodiments, R² is unsubstituted unbranched C₁₆₋₁₈ (e.g., C16, C17, C18) alkenyl. In certain embodiments, R² is unsubstituted unbranched C₁₆₋₁₈ (e.g., C16, C17, C18) alkynyl. When R² is C12-C21, the gamma lactone is C16-C25. When R² is C16-C21, the gamma lactone is C20-C25. When R² is C16-C18, the gamma lactone is C20-C22.

In certain embodiments, the product delta-lactone and/or product gamma-lactone do not comprise C═C═C. In certain embodiments, the product delta-lactone and/or product gamma-lactone do not comprise C≡C. In certain embodiments, the product delta-lactone and/or product gamma-lactone comprise only one C≡C. In certain embodiments, at least one double bond of the alkenyl is a Z double bond. In certain embodiments, each double bond of the alkenyl is a Z double bond. In certain embodiments, at least one double bond of the alkenyl is an E double bond. In certain embodiments, each double bond of the alkenyl is an E double bond. In certain embodiments, R² comprises only one double bond. In certain embodiments, R² comprises only two double bonds. In certain embodiments, R² comprises only three double bonds. In certain embodiments, R² comprises only four double bonds. In certain embodiments, R² comprises only five double bonds. In certain embodiments, R² comprises only six double bonds. In certain embodiments, each two double bonds of R², if present, are separated by two single bonds.

In each of Formulae (V) and (VI), the carbon atom marked with * as shown below is a chiral carbon atom:

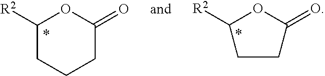

In certain embodiments, the chiral carbon atom is of the S configuration. In certain embodiments, the chiral carbon atom is of the R configuration.

In certain embodiments, the product delta-lactone is represented by the formula:

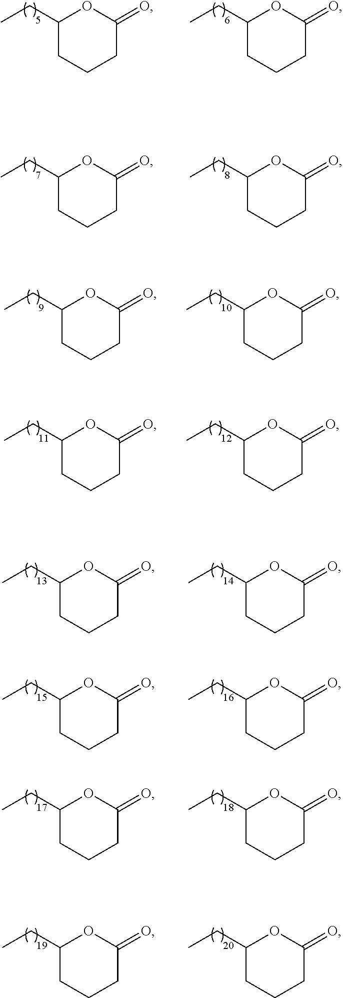

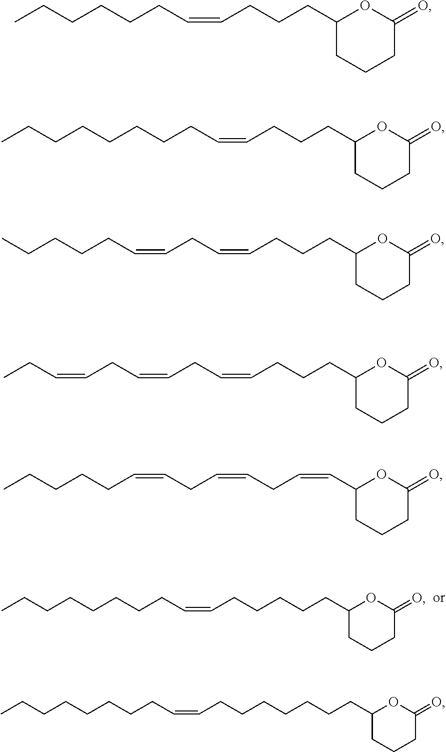

or a tautomer, isotopically labeled compound, solvate, polymorph, or co-crystal thereof; or a mixture thereof.

In certain embodiments, the product gamma-lactone is of the formula:

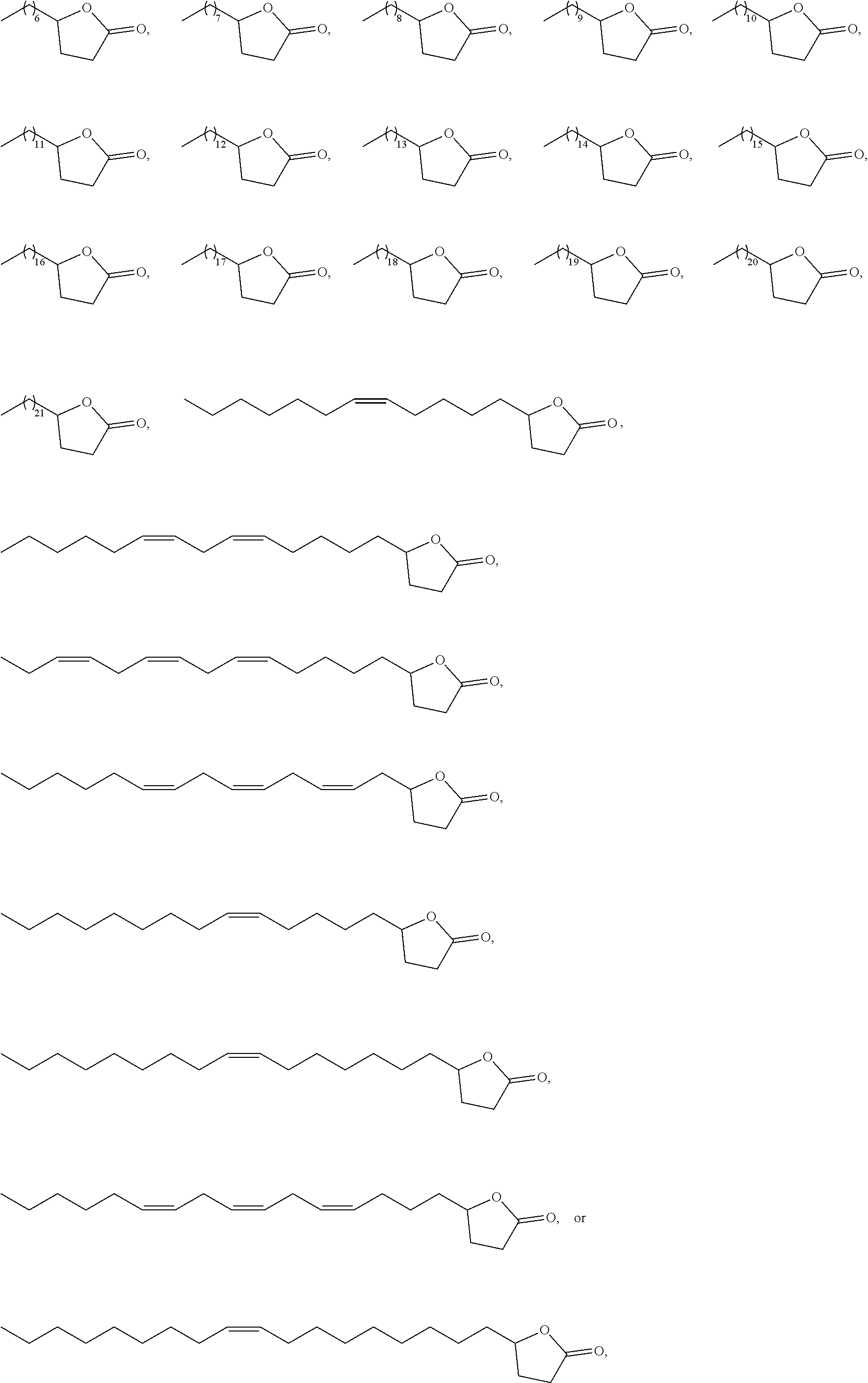

or a tautomer, isotopically labeled compound, solvate, polymorph, or co-crystal thereof; or a mixture thereof.

In certain embodiments, the product delta-lactone comprises a mixture of two or more delta-lactones described herein (e.g., delta-lactones of Formula (V) and/or delta-lactones of Formula (IV)), or tautomers, isotopically labeled compounds, solvates, polymorphs, or co-crystals thereof. In certain embodiments, the mixture is a mixture of the S- and R-enantiomers of otherwise the same delta-lactone, or tautomers, isotopically labeled compounds, solvates, polymorphs, or co-crystals thereof. In certain embodiments, the mixture is a racemic mixture of the S- and R-enantiomers of otherwise the same delta-lactone, or tautomers, isotopically labeled compounds, solvates, polymorphs, or co-crystals thereof.

In certain embodiments, the product gamma-lactone comprises a mixture of two or more gamma-lactones described herein (e.g., gamma-lactones of Formula (VI) and/or gamma-lactones of Formula (IV)), or tautomers, isotopically labeled compounds, solvates, polymorphs, or co-crystals thereof. In certain embodiments, the mixture is a mixture of the S- and R-enantiomers of otherwise the same gamma-lactone, or tautomers, isotopically labeled compounds, solvates, polymorphs, or co-crystals thereof. In certain embodiments, the mixture is a racemic mixture of the S- and R-enantiomers of otherwise the same gamma-lactone, or tautomers, isotopically labeled compounds, solvates, polymorphs, or co-crystals thereof.

In certain embodiments, the product delta-lactone is a delta-lactone of Formula (IV), or a tautomer, isotopically labeled compound, solvate, polymorph, or co-crystal thereof; or a mixture of two or more delta-lactones of Formula (IV), or tautomers, isotopically labeled compounds, solvates, polymorphs, or co-crystals thereof.

In certain embodiments, the product gamma-lactone is a gamma-lactone of Formula (IV), or a tautomer, isotopically labeled compound, solvate, polymorph, or co-crystal thereof; or a mixture of two or more gamma-lactones of Formula (IV), or tautomers, isotopically labeled compounds, solvates, polymorphs, or co-crystals thereof.

In some embodiments, there is provided a biosynthetic process yielding a product composition where the delta-lactone is not less than 50% (e.g., not less than 50%, not less than 55%, not less than 60%, not less than 65%, not less than 70%, not less than 75%, not less than 80%, not less than 85%, not less than 90%, not less than 95%, or not less than 99%) pure. Other components of the product composition may include additional lactones, for instance one or more gamma-lactones. In certain embodiments, the impurities in the product delta-lactone comprise one or more gamma-lactones. In certain embodiments, the impurities in the product gamma-lactone comprise one or more delta-lactones. In one non-limiting example, the substrate from which the delta-lactone is produced is lauric acid, a salt thereof, an alkyl ester thereof, a mono, di or triglyceride thereof or an unsubstituted monoalkyl or dialkyl amide thereof. In an embodiment of this example, the product delta-dodecalactone is at least 70% pure.

In representative embodiments, the biosynthetic process further comprises: (i) purifying a crude delta-lactone product; and, (ii) removing solvents under vacuum to provide a concentrated delta-lactone product. In representative embodiments, the biosynthetic process further comprises: (i) purifying a crude gamma-lactone product; and, (ii) removing solvents under vacuum to provide a concentrated gamma-lactone product. In one, non-limiting example, the crude product is purified by column chromatography. In another example, the crude product is purified by acid-base extraction. In a further example, said crude product is purified by vacuum distillation. In some embodiments, the method of production further comprises purifying the δ-dodecalactone using a semi-preparative high-pressure liquid chromatography (HPLC) process. In further embodiments, provided herein is a consumable item comprising a flavoring amount of one or more product delta-lactones. In further embodiments, provided herein is a consumable item comprising a flavoring amount of one or more product gamma-lactones. In exemplary embodiments, the consumable item is selected from the group consisting of beverages, confectioneries, bakery products, cookies, and chewing gums.

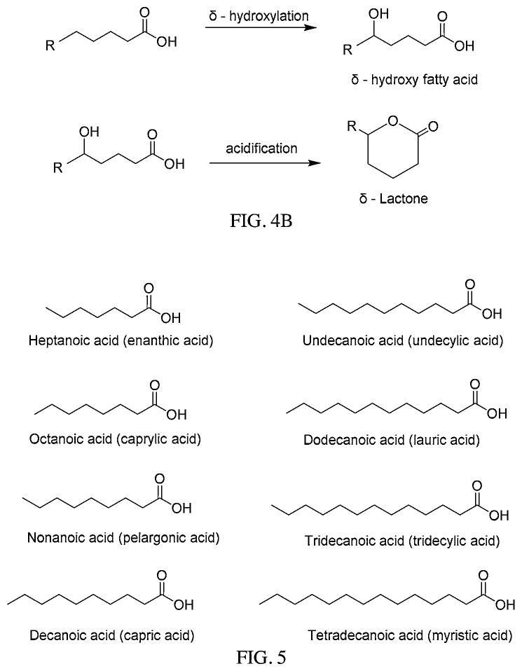

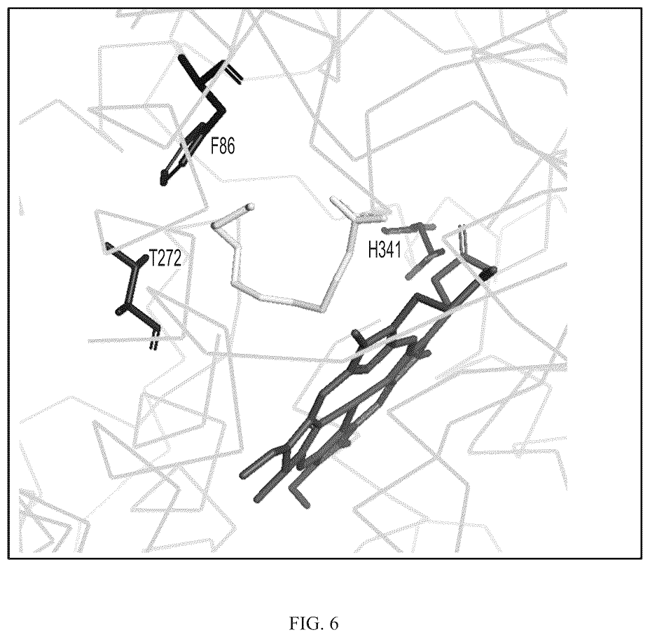

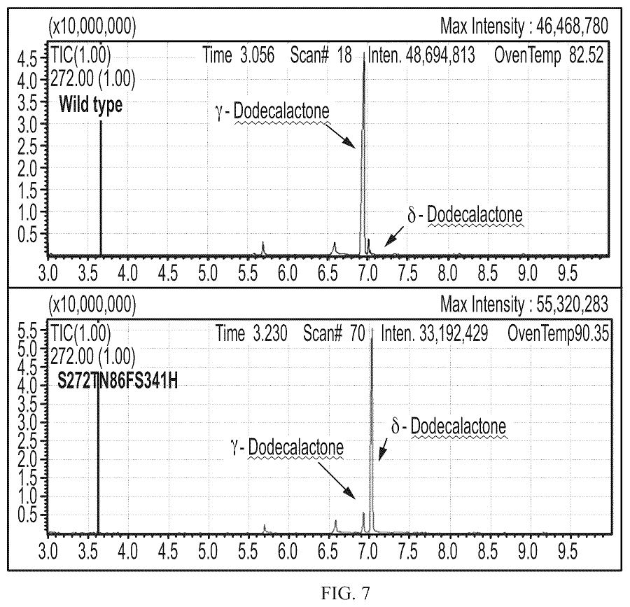

In one embodiment, the delta-lactone is produced by an in vivo bioconversion method. In some embodiments, the gamma-lactone is produced by an in vivo bioconversion method. A recombinant cellular system, for example E. coli cells hosting a mutant fungal P450 hydroxylase gene, is grown in a nutritious medium, then expression of the protein is induced by IPTG. After adding fatty acid or their precursors (FIG. 4A), the production of corresponding delta-lactones is detected by GC/MS. The lactones of interest are then formed and harvested (FIG. 4B). Besides E. coli, the cellular system may be formed from bacteria or yeasts belonging to any suitable genus of microorganisms which allows for the genetic transformation with the selected genes and thereafter the biosynthetic production of the desired delta-lactone from a substrate. Besides E. coli, the cellular system may be formed from bacteria or yeasts belonging to any suitable genus of microorganisms which allows for the genetic transformation with the selected genes and thereafter the biosynthetic production of the desired gamma-lactone from a substrate. Example bacterial genera include Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium, while typical yeast species include Saccharomyces; Zygosaccharomyces; Kluyveromyces; Candida; Streptomyces; Hansenula; Debaryomyces; Mucor; Pichia; Torulopsis; Aspergillus; and Arthrobotlys. Also contemplated are cellular systems formed from other organisms, e.g., recombinant algal or plant cells.

In other aspects, the disclosure provides a Cytochrome P450 recombinant gene comprising a DNA sequence having at least 80% (e.g., at least 80%, at least 85%, at least 90%, at least 95%, at least 95%, at least 97%, at least 98%, at least 99% or 100%) sequence identity to SEQ ID NO: 4, 6, 8, 10, or 12. In some embodiments, the amino acid sequence of the Cytochrome P450 recombinant polypeptide coded by the recombinant gene has at least 80% (e.g., at least 80%, at least 85%, at least 90%, at least 95%, at least 95%, at least 97%, at least 98%, at least 99% or 100%) identity to SEQ ID NO: 3, 5, 7, 9, or 11. The enzymatic product of the recombinant polypeptide includes a delta-lactone having a purity of not less than 50%, not less than 60%, not less than 65%, not less than 70%, or not less than 75%.

In terms of product/commercial utility many products containing delta-lactone are on the market in the United States and can be used in everything from perfumes, food and beverages, to pharmaceuticals. Products containing delta-lactones can be aerosols, liquids, or granular formulations. In terms of product/commercial utility many products containing gamma-lactone are on the market in the United States and can be used in everything from perfumes, food and beverages, to pharmaceuticals. Products containing gamma-lactones can be aerosols, liquids, or granular formulations.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

### Synthetic Biology

Standard recombinant DNA and molecular cloning techniques used here are well known in the art and are described, for example, by Sambrook, J., Fritsch, E. F. and Maniatis, T. MOLECULAR CLONING: A LABORATORY MANUAL, 2nd ed.; Cold Spring Harbor Laboratory: Cold Spring Harbor, N.Y., 1989 (hereinafter “Maniatis”); and by Silhavy, T. J., Bennan, M. L. and Enquist, L. W. EXPERIMENTS WITH GENE FUSIONS; Cold Spring Harbor Laboratory: Cold Spring Harbor, N.Y., 1984; and by Ausubel, F. M. et al., IN CURRENT PROTOCOLS IN MOLECULAR BIOLOGY, published by GREENE PUBLISHING AND WILEY-INTERSCIENCE, 1987; (the entirety of each of which is hereby incorporated herein by reference).

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

The disclosure will be more fully understood upon consideration of the following non-limiting Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Bacterial Production Systems

Expression of proteins in prokaryotes is most often carried out in a bacterial host cell with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such vectors are within the scope of the present disclosure.

In an embodiment, the expression vector includes those genetic elements for expression of the recombinant polypeptide in bacterial cells. The elements for transcription and translation in the bacterial cell can include a promoter, a coding region for the protein complex, and a transcriptional terminator.

A person of ordinary skill in the art will be aware of the molecular biology techniques available for the preparation of expression vectors. A polynucleotide used for incorporation into the expression vector of the subject technology, as described above, can be prepared by routine techniques such as polymerase chain reaction (PCR).

A number of molecular biology techniques have been developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In an alternative embodiment, synthetic linkers containing one or more restriction sites provide are used to operably link the polynucleotide of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill-in recessed 3′-ends with their polymerizing activities, thereby generating blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In an embodiment, in order to isolate and/or modify the polynucleotide of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In an embodiment, a polynucleotide for incorporation into an expression vector of the subject technology is prepared by the use of PCR using appropriate oligonucleotide primers. The coding region can be amplified, whilst the primers themselves become incorporated into the amplified sequence product. In an embodiment, the amplification primers contain restriction endonuclease recognition sites, which allow the amplified sequence product to be cloned into an appropriate vector.

The expression vectors can be introduced into plant or microbial host cells by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, cells transfected with an expression vector of the subject technology can be cultured to produce polypeptides described herein. Cells can be examined for the presence of the expression vector DNA by techniques well known in the art.

The host cells can contain a single copy of the expression vector described previously, or alternatively, multiple copies of the expression vector.

In some embodiments, the transformed cell is an animal cell, an insect cell, a plant cell, an algal cell, a fungal cell, or a yeast cell. In some embodiments, the cell is a plant cell selected from the group consisting of: canola plant cell, a rapeseed plant cell, a palm plant cell, a sunflower plant cell, a cotton plant cell, a corn plant cell, a peanut plant cell, a flax plant cell, a sesame plant cell, a soybean plant cell, and a petunia plant cell.

Microbial host cell expression systems and expression vectors containing regulatory sequences that direct high-level expression of foreign proteins are well known to those skilled in the art. Any of these could be used to construct vectors for expression of the recombinant polypeptide of the subjection technology in a microbial host cell. These vectors could then be introduced into appropriate microorganisms via transformation to allow for high level expression of the recombinant polypeptide of the subject technology.

Vectors or cassettes useful for the transformation of suitable microbial host cells are well known in the art. Typically the vector or cassette contains sequences directing transcription and translation of the relevant polynucleotide, a selectable marker, and sequences allowing autonomous replication or chromosomal integration. Suitable vectors comprise a region 5′ of the polynucleotide which harbors transcriptional initiation controls and a region 3′ of the DNA fragment which controls transcriptional termination. It is preferred for both control regions to be derived from genes homologous to the transformed host cell, although it is to be understood that such control regions need not be derived from the genes native to the specific species chosen as a host.

Initiation control regions or promoters, which are useful to drive expression of the recombinant polypeptide in the desired microbial host cell are numerous and familiar to those skilled in the art. Virtually any promoter capable of driving these genes is suitable for the subject technology including but not limited to CYCI, HIS3, GALI, GALIO, ADHI, PGK, PH05, GAPDH, ADCI, TRPI, URA3, LEU2, ENO, TPI (useful for expression in Saccharomyces); AOXI (useful for expression in Pichia); and lac, trp, JPL, IPR, T7, tac, and tre (useful for expression in Escherichia coli).

Termination control regions may also be derived from various genes native to the microbial hosts. A termination site optionally may be included for the microbial hosts described herein.

In plant cells, the expression vectors of the subject technology can include a coding region operably linked to promoters capable of directing expression of the recombinant polypeptide of the subject technology in the desired tissues at the desired stage of development. For reasons of convenience, the polynucleotides to be expressed may comprise promoter sequences and translation leader sequences derived from the same polynucleotide. 3′ non-coding sequences encoding transcription termination signals can also be present. The expression vectors may also comprise one or more introns in order to facilitate polynucleotide expression.

For plant host cells, any combination of any promoter and any terminator capable of inducing expression of a coding region may be used in the vector sequences of the subject technology. Some suitable examples of promoters and terminators include those from nopaline synthase (nos), octopine synthase (ocs) and cauliflower mosaic virus (CaMV) genes. One type of efficient plant promoter that may be used is a high-level plant promoter. Such promoters, in operable linkage with an expression vector of the subject technology should be capable of promoting the expression of the vector. High level plant promoters that may be used in the subject technology include the promoter of the small subunit (s) of the ribulose-1,5-bisphosphate carboxylase for example from soybean (Berry-Lowe et al., J. MOLECULAR AND APP. GEN., 1:483 498 (1982), the entirety of which is hereby incorporated herein to the extent it is consistent herewith), and the promoter of the chlorophyll binding protein. These two promoters are known to be light-induced in plant cells (see, for example, GENETIC ENGINEERING OF PLANTS, AN AGRICULTURAL PERSPECTIVE, A. Cashmore, Plenum, N.Y. (1983), pages 29 38; Coruzzi, G. et al., The Journal of Biological CHEMISTRY, 258: 1399 (1983), and Dunsmuir, P. et al., JOURNAL OF MOLECULAR AND APPLIED GENETICS, 2:285 (1983), each of which is hereby incorporated herein by reference to the extent they are consistent herewith).

One with skill in the art will recognize that the lactone composition(s) produced by the methods described herein can be further purified and mixed with other lactones, flavors, or scents to obtain a desired composition for use in a variety of consumer products or foods.

For example, the δ-dodecalactone composition described herein can be included in food products (such as beverages, soft drinks, ice cream, dairy products, confectioneries, cereals, chewing gum, baked goods, etc.), dietary supplements, medical nutrition, as well as pharmaceutical products to give desired flavor characteristics for a variety of desirable flavors. Other lactones produced by the methods herein or produced at the same time through the activity of the P450 hydroxylating enzyme of the present disclosure can be purified and provided alone or together for a defined flavor composition, food or fragrance.

### Analysis of Sequence Similarity Using Identity Scoring

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this disclosure “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10; Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981, Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the disclosure is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity of the Blu1 and Cytochrome P450 genes of the current disclosure are capable of directing the production of a variety of δ-dodecalactones and have a substantial percent sequence identity to the polynucleotide sequences provided herein and are encompassed within the scope of this disclosure.

### Identity and Similarity

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

As is evident from the foregoing description, certain aspects of the present disclosure are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present disclosure.

Moreover, unless defined otherwise, all technical and scientific terms used herein have the same meaning as commonly understood by one of ordinary skill in the art to which the disclosure belongs. Although any methods and materials similar to or equivalent to or those described herein can be used in the practice or testing of the present disclosure, the preferred methods and materials are described above.

In another aspect, the present disclosure provides a gamma- or delta-lactone represented by Formula (IV) or (1):

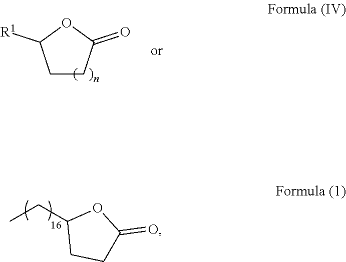

or a tautomer, isotopically labeled compound, solvate, polymorph, or co-crystal thereof, wherein:
n is 1 or 2; and
R¹ is unsubstituted unbranched C₇₋₁₈ alkenyl, wherein each double bond of the unsubstituted unbranched C₇₋₁₈ alkenyl is a Z double bond;
provided that the gamma- or delta-lactone does not comprise C═C═C or C≡C and is not represented by the formula:

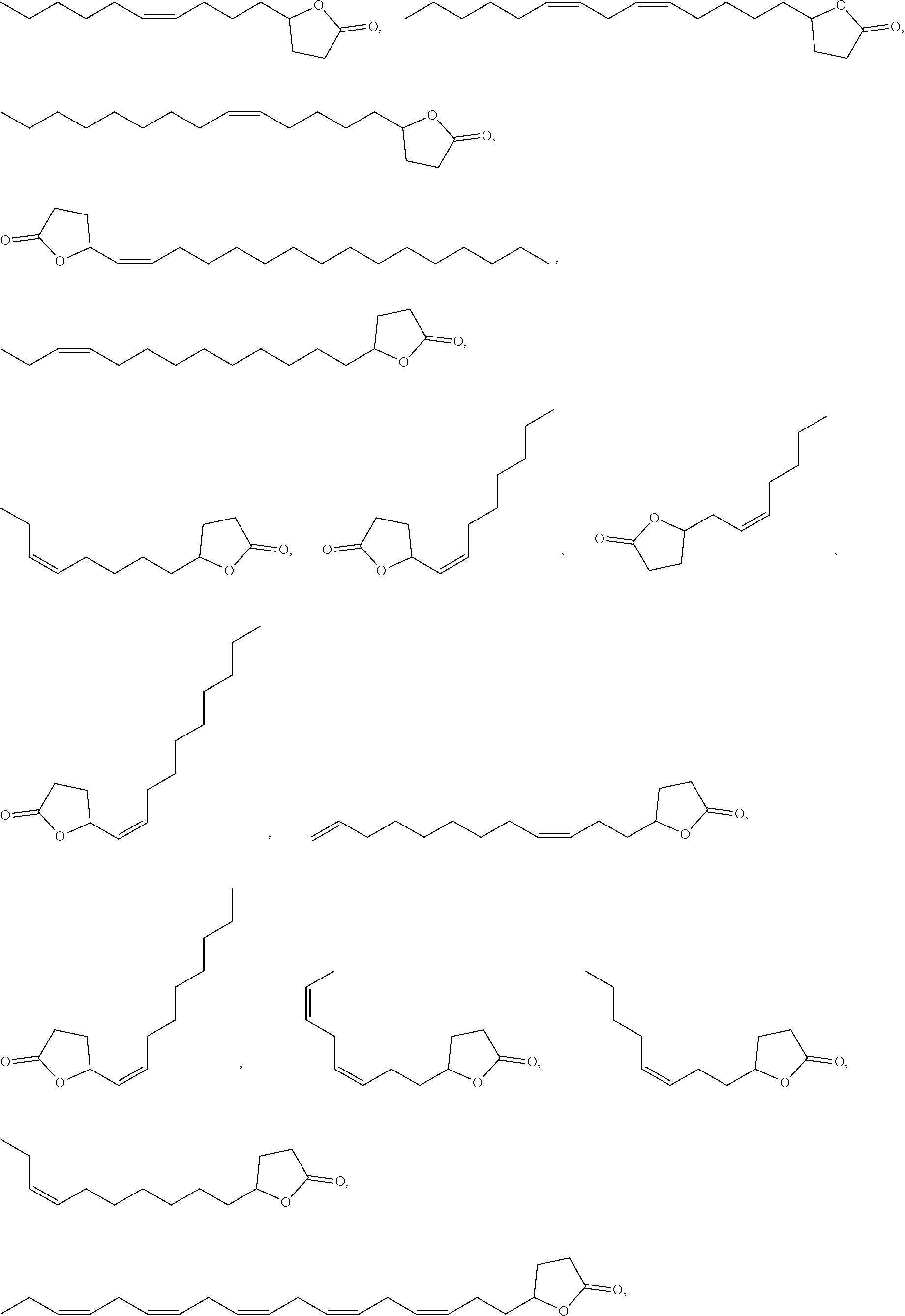

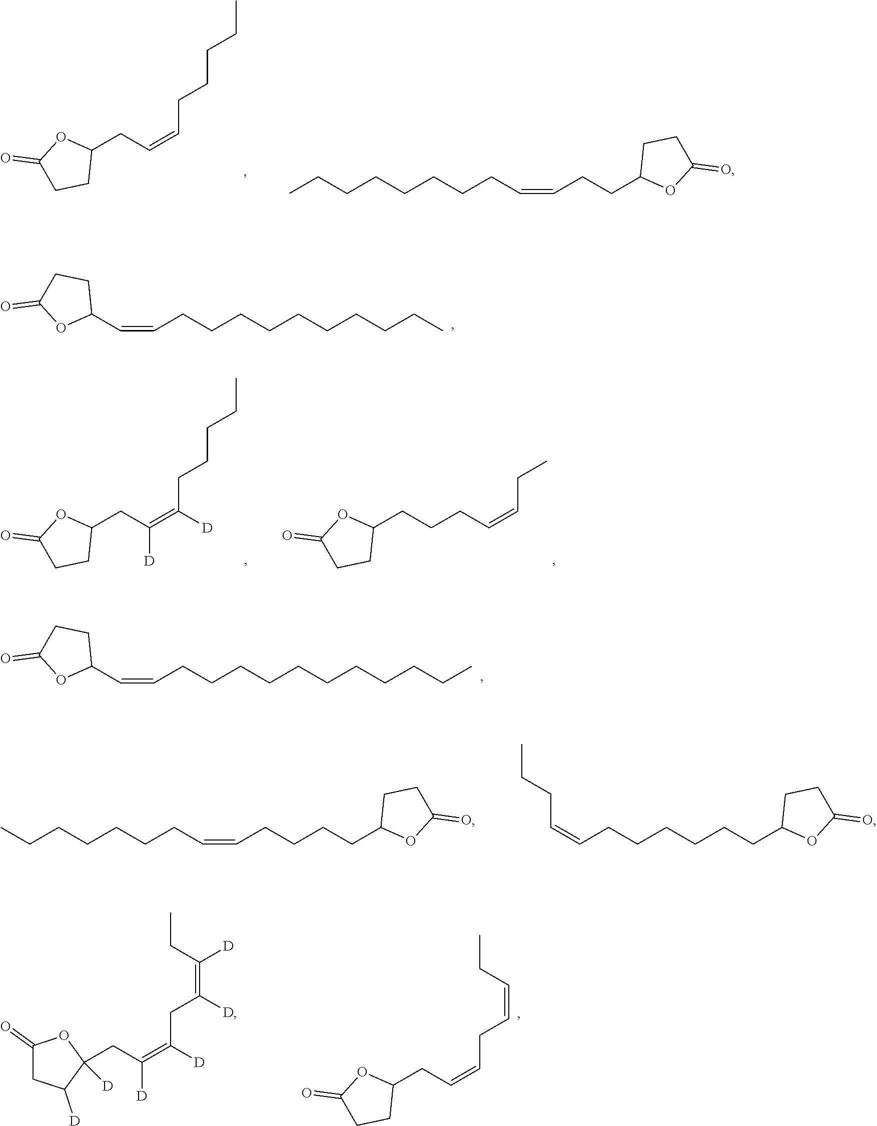

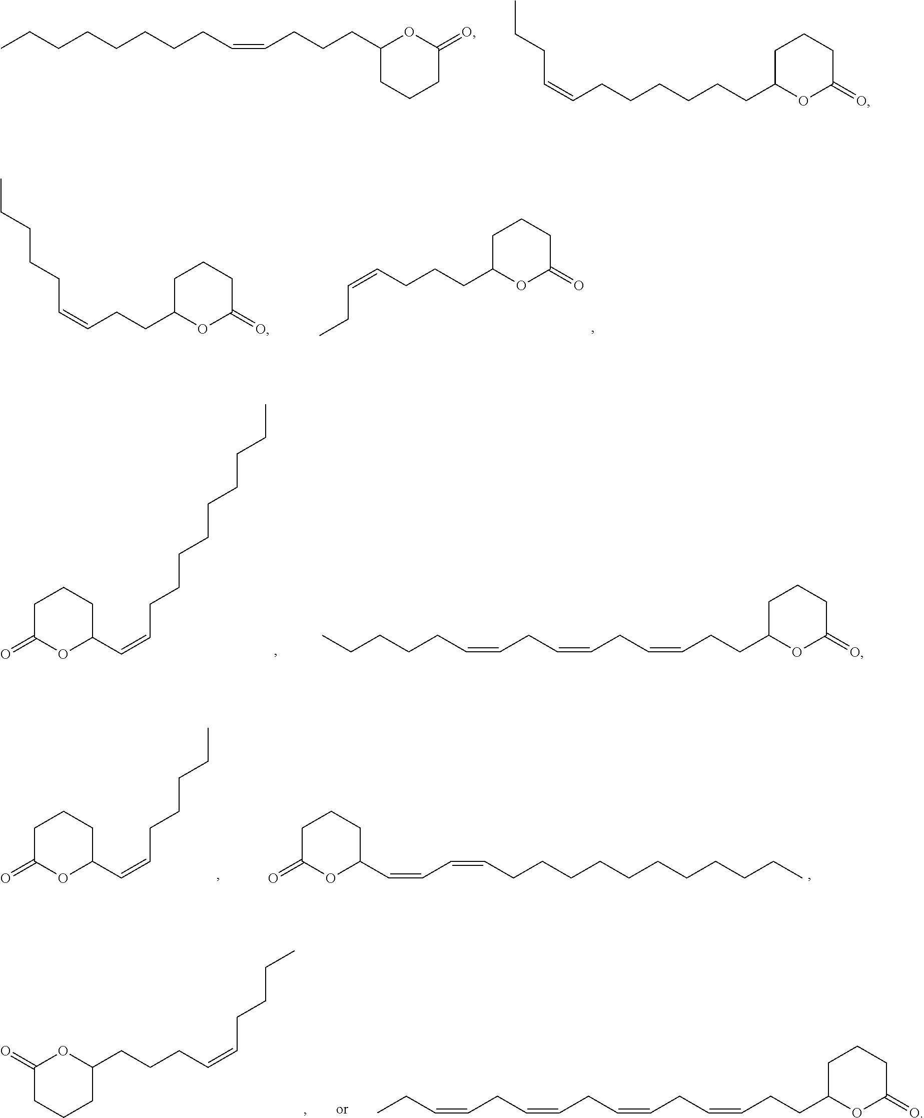

In certain embodiments, R¹ is unsubstituted unbranched C₇₋₉ alkenyl. In certain embodiments, R¹ is unsubstituted unbranched C₁₀₋₁₂ alkenyl. In certain embodiments, R¹ is unsubstituted unbranched C₁₃₋₁₅ alkenyl. In certain embodiments, R¹ is unsubstituted unbranched C₁₆₋₁₈ alkenyl. In certain embodiments, R¹ is unsubstituted unbranched C₁₋₇ alkenyl. In certain embodiments, R¹ comprises only one double bond. In certain embodiments, R¹ comprises only two double bonds. In certain embodiments, R¹ comprises only three double bonds. In certain embodiments, R¹ comprises only four double bonds. In certain embodiments, R¹ comprises only five double bonds. In certain embodiments, R¹ comprises only six double bonds. In certain embodiments, each two double bonds of R¹, if present, are separated by two single bonds.

In any one of Formulae (IV) and (1), the carbon atom marked with * as shown below is a chiral carbon atom:

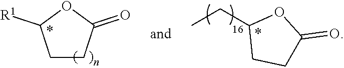

In certain embodiments, the chiral carbon atom is of the S configuration. In certain embodiments, the chiral carbon atom is of the R configuration.

In certain embodiments, n is 1. In certain embodiments, the gamma-lactone is represented by the formula:

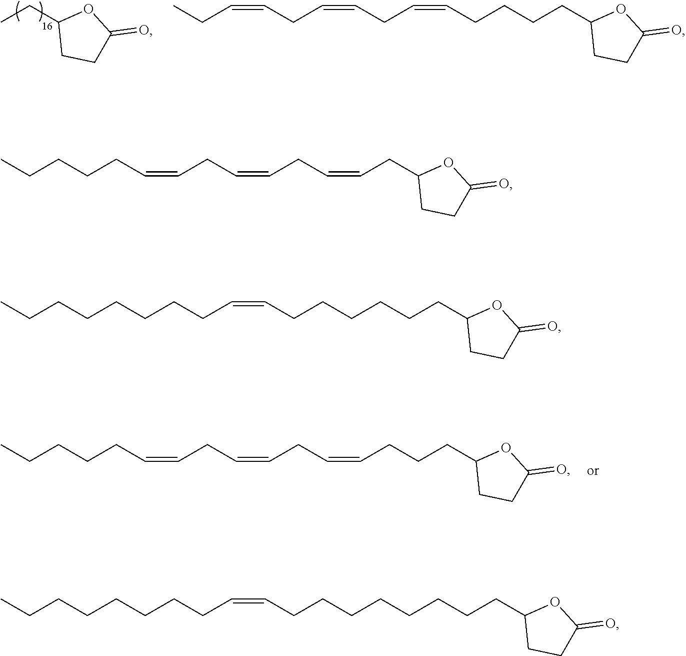

or a tautomer, isotopically labeled compound, solvate, polymorph, or co-crystal thereof.

In certain embodiments, n is 2. In certain embodiments, the delta-lactone is represented by the formula:

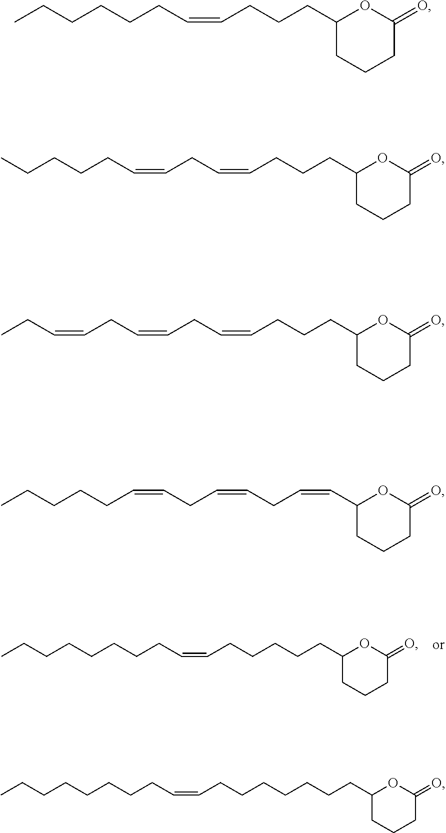 [SMILES: CCCCCC/C=C\CCCC1CCCC(=O)O1 ; CCCCC/C=C\C/C=C\CCCC1CCCC(=O)O1 ; CC/C=C\C/C=C\C/C=C\CCCC1CCCC(=O)O1 ; CCCCC/C=C\C/C=C\C/C=C\C1CCCC(=O)O1 ; CCCCCCCC/C=C\CCCCCC1CCCC(=O)O1 ; CCCCCCCC/C=C\CCCCCCCC1CCCC(=O)O1]

or a tautomer, isotopically labeled compound, solvate, polymorph, or co-crystal thereof.

In certain embodiments, the delta- or gamma-lactone, or a tautomer, isotopically labeled compound, solvate, polymorph, or co-crystal thereof, having a purity between 50% and 60%, between 60% and 70%, between 70% and 80%, between 80% and 90%, between 90% and 95%, between 95% and 99%, or between 99% and 99.9%. In certain embodiments, an impurity in the delta- or gamma-lactone, or a tautomer, isotopically labeled compound, solvate, polymorph, or co-crystal thereof, is the opposite enantiomer of the delta- or gamma-lactone, or a tautomer, isotopically labeled compound, solvate, polymorph, or co-crystal thereof. In certain embodiments, the opposite enantiomer of the delta- or gamma-lactone, or a tautomer, isotopically labeled compound, solvate, polymorph, or co-crystal thereof, is not considered to be an impurity in the delta- or gamma-lactone, or a tautomer, isotopically labeled compound, solvate, polymorph, or co-crystal thereof.

In another aspect, the present disclosure provides a mixture of two or more delta- or gamma-lactones, or tautomers, isotopically labeled compounds, solvates, polymorphs, or co-crystals thereof. In certain embodiments, the mixture is a mixture of the S- and R-enantiomers of otherwise the same delta- or gamma-lactone, or tautomers, isotopically labeled compounds, solvates, polymorphs, or co-crystals thereof. In certain embodiments, the mixture is a racemic mixture of the S- and R-enantiomers of otherwise the same delta- or gamma-lactone, or tautomers, isotopically labeled compounds, solvates, polymorphs, or co-crystals thereof. In certain embodiments, the mixture is a mixture of the S- and R-enantiomers of

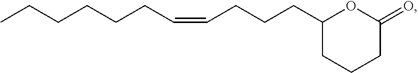 [SMILES: CCCCCC/C=C\CCCC1CCCC(=O)O1]

or tautomers, isotopically labeled compounds, solvates, polymorphs, or co-crystals thereof. In certain embodiments, the mixture is a mixture of the S- and R-enantiomers of

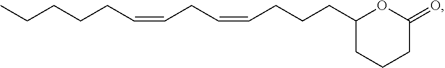 [SMILES: CCCCC/C=C\C/C=C\CCCC1CCCC(=O)O1]

or tautomers, isotopically labeled compounds, solvates, polymorphs, or co-crystals thereof. In certain embodiments, the mixture is a mixture of the S- and R-enantiomers of

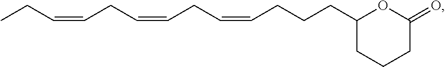 [SMILES: CC/C=C\C/C=C\C/C=C\CCCC1CCCC(=O)O1]

or tautomers, isotopically labeled compounds, solvates, polymorphs, or co-crystals thereof. In certain embodiments, the mixture is a mixture of the S- and R-enantiomers of

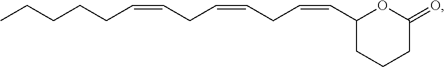 [SMILES: CCCCC/C=C\C/C=C\C/C=C\C1CCCC(=O)O1]

or tautomers, isotopically labeled compounds, solvates, polymorphs, or co-crystals thereof. In certain embodiments, the mixture is a mixture of the S- and R-enantiomers of

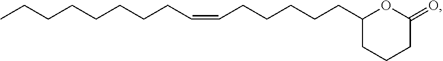 [SMILES: CCCCCCCC/C=C\CCCCCC1CCCC(=O)O1]

or tautomers, isotopically labeled compounds, solvates, polymorphs, or co-crystals thereof. In certain embodiments, the mixture is a mixture of the S- and R-enantiomers of

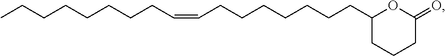 [SMILES: CCCCCCCC/C=C\CCCCCCCC1CCCC(=O)O1]

or tautomers, isotopically labeled compounds, solvates, polymorphs, or co-crystals thereof. In certain embodiments, the mixture is a mixture of the S- and R-enantiomers of

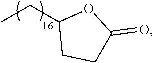

or tautomers, isotopically labeled compounds, solvates, polymorphs, or co-crystals thereof.

In certain embodiments, the mixture is a mixture of the S- and R-enantiomers of

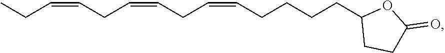 [SMILES: CC/C=C\C/C=C\C/C=C\CCCCC1CCC(=O)O1]

or tautomers, isotopically labeled compounds, solvates, polymorphs, or co-crystals thereof.

In certain embodiments, the mixture is a mixture of the S- and R-enantiomers of

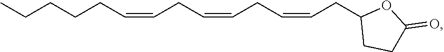 [SMILES: CCCCC/C=C\C/C=C\C/C=C\CC1CCC(=O)O1]

or tautomers, isotopically labeled compounds, solvates, polymorphs, or co-crystals thereof.

In certain embodiments, the mixture is a mixture of the S- and R-enantiomers of

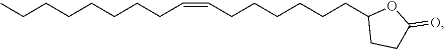 [SMILES: CCCCCCCC/C=C\CCCCCCC1CCC(=O)O1]

or tautomers, isotopically labeled compounds, solvates, polymorphs, or co-crystals thereof.

In certain embodiments, the mixture is a mixture of the S- and R-enantiomers of

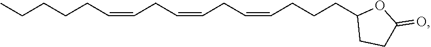 [SMILES: CCCCC/C=C\C/C=C\C/C=C\CCCC1CCC(=O)O1]

or tautomers, isotopically labeled compounds, solvates, polymorphs, or co-crystals thereof.

In certain embodiments, the mixture is a mixture of the S- and R-enantiomers of

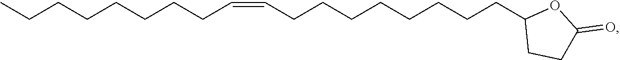 [SMILES: CCCCCCCC/C=C\CCCCCCCCC1CCC(=O)O1]

or tautomers, isotopically labeled compounds, solvates, polymorphs, or co-crystals thereof.

In another aspect, the present disclosure provides a composition comprising the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; or the mixture.

In certain embodiments, the product delta-lactone comprised in the composition is the product delta-lactone represented by Formula (V). In certain embodiments, the delta-lactone comprised in the composition is the delta-lactone represented by Formula (IV). In certain embodiments, the product gamma-lactone comprised in the composition is the product gamma-lactone represented by Formula (VI). In certain embodiments, the gamma-lactone comprised in the composition is the gamma-lactone represented by Formula (IV). In certain embodiments, the composition further comprises an excipient. In certain embodiments, the excipient is a pharmaceutically acceptable excipient. In certain embodiments, the excipient is a cosmetically acceptable excipient. In certain embodiments, the excipient is a nutraceutically acceptable excipient. In certain embodiments, the composition further comprises a pharmaceutically acceptable excipient, cosmetically acceptable excipient, or nutraceutically acceptable excipient.

In another aspect, the present disclosure provides a delta-lactone, or a tautomer, isotopically labeled compound, solvate, polymorph, or co-crystal thereof, produced by the bioconversion method described herein. In another aspect, the present disclosure provides a delta-lactone, or a tautomer, isotopically labeled compound, solvate, polymorph, or co-crystal thereof, produced by the bioenzymatic method described herein. In another aspect, the present disclosure provides a gamma-lactone, or a tautomer, isotopically labeled compound, solvate, polymorph, or co-crystal thereof, produced by the bioconversion method described herein. In another aspect, the present disclosure provides a gamma-lactone, or a tautomer, isotopically labeled compound, solvate, polymorph, or co-crystal thereof, produced by the bioenzymatic method described herein.

In another aspect, the present disclosure also provides a kit comprising:
the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; the mixture; or the composition; and
instructions for using the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; the mixture; or the composition.

In certain embodiments, the kit comprises a first container, wherein the first container comprises the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; the mixture; or the composition. In some embodiments, the kit further comprises a second container. In certain embodiments, the second container comprises an excipient (e.g., pharmaceutically acceptable excipient, cosmetically acceptable excipient, or nutraceutically acceptable excipient). In certain embodiments, the second container comprises the instructions. In certain embodiments, each of the first and second containers is independently a vial, ampule, bottle, syringe, dispenser package, tube, or box.

In another aspect, the present disclosure also provides a method of altering the flavor of a food, drink, oral dietary supplement, or oral pharmaceutical product comprising adding an effective amount of: the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; the mixture; or the composition, to the food, drink, oral dietary supplement, or oral pharmaceutical product, or to a raw or intermediate material for producing the food, drink, oral dietary supplement, or oral pharmaceutical product. In certain embodiments, the food is a meat product. In certain embodiments, the meat product is a chicken product, turkey product, duck product, goose product, quill product, pheasant product, beef product, veal product, lamb product, mutton product, pork product, venison product, rabbit product, wild boar product, or bison product. In certain embodiments, the meat product is a processed meat product. In certain embodiments, the food or drink is a dairy product. In certain embodiments, the food or drink is milk, cheese, butter, cream, ice cream, or yogurt. In certain embodiments, the food is a sauce, cereal, chocolate, cocoa, fish product, potato, nut product, popcorn, confectionery, chewing gum, or baked product. In certain embodiments, the drink is a coffee, tea, liquor, wine, or beer. In certain embodiments, the oral pharmaceutical product is a therapeutical product, prophylactic product, or diagnostic product, each of which is suitable for oral administration. In certain embodiments, the effective amount is effective in enhancing fatty flavor.

A method of altering the fatty feeling of a cosmetic product comprising adding an effective amount of: the product delta-lactone, or a tautomer, isotopically labeled compound, solvate, polymorph, or co-crystal thereof; the product gamma-lactone, or a tautomer, isotopically labeled compound, solvate, polymorph, or co-crystal thereof; the delta-lactone, or a tautomer, isotopically labeled compound, solvate, polymorph, or co-crystal thereof; the gamma-lactone, or a tautomer, isotopically labeled compound, solvate, polymorph, or co-crystal thereof; the mixture; or the composition, to the cosmetic product, or to a raw or intermediate material for producing the cosmetic product. In certain embodiments, the cosmetic product is a baby product, bath preparation, eye makeup preparation, fragrance preparation, non-coloring hair preparation, hair coloring preparation, non-eye makeup preparation, manicuring preparation, oral hygiene product, personal cleanliness, shaving preparation, skin care preparation (e.g., cream, lotion, powder, or spray), or suntan preparation. In certain embodiments, the effective amount is effective in enhancing fatty feeling.

In certain embodiments, the product delta-lactone, or a tautomer, isotopically labeled compound, solvate, polymorph, or co-crystal thereof, is the product delta-lactone; the product gamma-lactone, or a tautomer, isotopically labeled compound, solvate, polymorph, or co-crystal thereof, is the product gamma-lactone; the delta-lactone, or a tautomer, isotopically labeled compound, solvate, polymorph, or co-crystal thereof, is the delta-lactone; and the gamma-lactone, or a tautomer, isotopically labeled compound, solvate, polymorph, or co-crystal thereof, is the gamma-lactone (e.g., not a tautomer, isotopically labeled compound, solvate, polymorph, or co-crystal thereof).

### EXAMPLES

The subject technology is further defined in the following Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Example 1: Modeling and Structure-Based Docking and Mutant Library Design

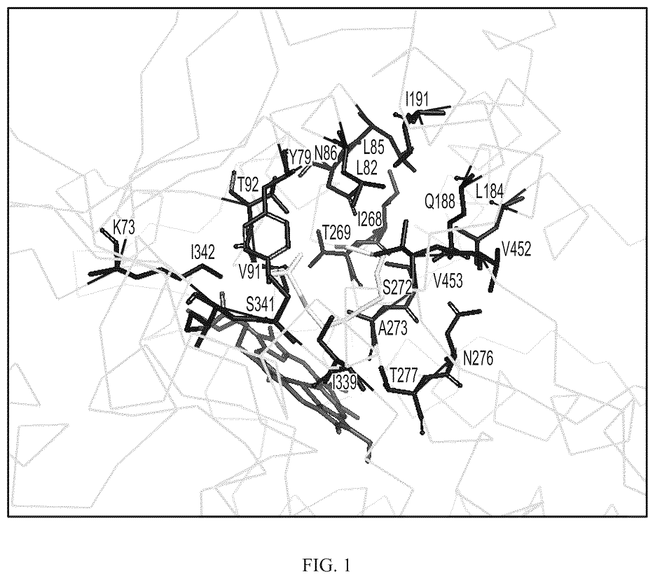

Modeling and docking experiments were carried out using ICM (integrated catchment modeling) modeling and docking software programs (Molsoft, San Diego, California). Multiple stack conformations were selected based on the docking energies and the rmsd values (root-mean-square deviation of atomic positions, or the average distance between backbone atoms of superimposed enzymes) of the enzyme-substrate complex, and binding energies were calculated using TCM script. For docking studies, the lauric acid substrate was docked into a Mucor ambiguus P450 modeling structure (FIG. 1). Based on the docking results, a total of 21 possible binding sites, including K73, Y79, L82, L85, N86, V91, T92, L184, Q188, I191, I268, T269, S272, A273, N276, T277, 1339, S341, 1342, V452, and V453 were identified as potential residues that form a binding pocket for lauric acid and for further investigation.

### Example 2: Cloning of Wild Type Enzyme and Variants

The wild type MaP450 gene from Mucor ambiguus (SEQ ID NO: 1) was cloned into a pET-16b-(+) vector (Novagen, Madison, Wisconsin). Based on the docking results described in Example 1, rational-design based mutagenesis was performed at sites K73, Y79, L82, L85, N86, V91, T92, L184, Q188, I191, I268, T269, S272, A273, N276, T277, 1339, S341, 1342, V452, and V453 of MaP450 by following the QuikChange site-directed mutagenesis strategy (STRATAgene, La Jolla, CA) using different primers (see Table 1). The QuikChange PCR products were examined by agarose gel electrophoresis and then 20 μl of PCR products were digested with 1 μl Dpnl (New England Biolabs, Ipswich, Massachusetts) at 37° C. for 1 hour to remove the template plasmids. Aliquots of 2 μl of digestive products were transformed into BL21(DE3)-competent E. coli cells (New England Biolabs) and inoculated on Luria-Bertani (LB) agar plates containing carbenicillin. The quality of the library was confirmed by DNA sequencing; a total of 221 mutants were screened.

TABLE 1
Primers

| Site | Primer | SEQ ID NO: |
|---|---|---|
| K73A_p1 | atcgctatacacgctttcgatttctgcggtgaaatattcattatcttcacaa | 13 |
| K73A_p2 | ttgtgaagataatgaatatttcaccgcagaaatcgaaagcgtgtatagcgat | 14 |
| K73C_p1 | cagatcgctatacacgctttcgatttcgcaggtgaaatattcattatcttcacaaat | 15 |
| K73C_p2 | atttgtgaagataatgaatatttcacctgcgaaatcgaaagcgtgtatagcgatctg | 16 |
| K73D_p1 | atcgctatacacgctttcgatttcatcggtgaaatattcattatcttcaca | 17 |
| K73D_p2 | tgtgaagataatgaatatttcaccgatgaaatcgaaagcgtgtatagcgat | 18 |
| K73G_p1 | atcgctatacacgctttcgatttctccggtgaaatattcattatcttcacaa | 19 |
| K73G_p2 | ttgtgaagataatgaatatttcaccggagaaatcgaaagcgtgtatagcgat | 20 |
| K73L_p1 | atcgctatacacgctttcgatttctaaggtgaaatattcattatcttcacaa | 21 |
| K73L_p2 | ttgtgaagataatgaatatttcaccttagaaatcgaaagcgtgtatagcgat | 22 |
| K73N_p1 | tcgctatacacgctttcgatttcattggtgaaatattcattatcttc | 23 |
| K73N_p2 | gaagataatgaatatttcaccaatgaaatcgaaagcgtgtatagcga | 24 |
| K73Q_p1 | atcgctatacacgctttcgatttcctgggtgaaatattcattatcttcaca | 25 |
| K73Q_p2 | tgtgaagataatgaatatttcacccaggaaatcgaaagcgtgtatagcgat | 26 |
| K73R_p1 | atacacgctttcgatttctctggtgaaatattcattatcttcacaaatg | 27 |
| K73R_p2 | catttgtgaagataatgaatatttcaccagagaaatcgaaagcgtgtat | 28 |
| K73S_p1 | gatcgctatacacgctttcgatttcgctggtgaaatattcattatcttcaca | 29 |
| K73S_p2 | tgtgaagataatgaatatttcaccagcgaaatcgaaagcgtgtatagcgatc | 30 |
| K73T_p1 | gatcgctatacacgctttcgatttccgtggtgaaatattcattatcttcaca | 31 |
| K73T_p2 | tgtgaagataatgaatatttcaccacggaaatcgaaagcgtgtatagcgatc | 32 |
| K73Y_p1 | atcgctatacacgctttcgatttcataggtgaaatattcattatcttcaca | 33 |
| K73Y_p2 | tgtgaagataatgaatatttcacctatgaaatcgaaagcgtgtatagcgat | 34 |
| Y79A_p1 | tcagaattgccagatcgctagccacgctttcgatttctttgg | 35 |
| Y79A_p2 | ccaaagaaatcgaaagcgtggctagcgatctggcaattctga | 36 |
| Y79C_p1 | cagaattgccagatcgctacacacgctttcgatttcttt | 37 |
| Y79C_p2 | aaagaaatcgaaagcgtgtgtagcgatctggcaattctg | 38 |
| Y79D_p1 | agaattgccagatcgctatccacgctttcgatttctttg | 39 |
| Y79D_p2 | caaagaaatcgaaagcgtggatagcgatctggcaattct | 40 |
| Y79G_p1 | tcagaattgccagatcgctacccacgctttcgatttctttgg | 41 |
| Y79G_p2 | ccaaagaaatcgaaagcgtgggtagcgatctggcaattctga | 42 |
| Y79K_p1 | ttcagaattgccagatcgctcttcacgctttcgatttctttgg | 43 |
| Y79K_p2 | ccaaagaaatcgaaagcgtgaagagcgatctggcaattctgaa | 44 |
| Y79L_p1 | gaattgccagatcgcttaacacgctttcgatttctttggtgaaatattc | 45 |
| Y79L_p2 | gaatatttcaccaaagaaatcgaaagcgtgttaagcgatctggcaattc | 46 |
| Y79N_p1 | agaattgccagatcgctattcacgctttcgatttctttg | 47 |
| Y79N_p2 | caaagaaatcgaaagcgtgaatagcgatctggcaattct | 48 |
| Y79Q_p1 | ttcagaattgccagatcgctctgcacgctttcgatttctttgg | 49 |
| Y79Q_p2 | ccaaagaaatcgaaagcgtgcagagcgatctggcaattctgaa | 50 |
| Y79R_p1 | tcagaattgccagatcgctacgcacgctttcgatttctttgg | 51 |
| Y79R_p2 | ccaaagaaatcgaaagcgtgcgtagcgatctggcaattctga | 52 |
| Y79S_p1 | tcagaattgccagatcgctactcacgctttcgatttctttgg | 53 |
| Y79S_p2 | ccaaagaaatcgaaagcgtgagtagcgatctggcaattctga | 54 |
| Y79T_p1 | tcagaattgccagatcgctagtcacgctttcgatttctttgg | 55 |
| Y79T_p2 | ccaaagaaatcgaaagcgtgactagcgatctggcaattctga | 56 |
| L82A_p1 | gaccattcagaattgccgcatcgctatacacgctttcgattt | 57 |
| L82A_p2 | aaatcgaaagcgtgtatagcgatgcggcaattctgaatggtc | 58 |
| L82D_p1 | cacgaccattcagaattgcatcatcgctatacacgctttcgatttc | 59 |
| L82D_p2 | gaaatcgaaagcgtgtatagcgatgatgcaattctgaatggtcgtg | 60 |
| L82Q_p1 | gaccattcagaattgcctgatcgctatacacgctt | 61 |
| L82Q_p2 | aagcgtgtatagcgatcaggcaattctgaatggtc | 62 |
| L85A_p1 | gaccacgaccattcgcaattgccagatcgctatacacgct | 63 |
| L85A_p2 | agcgtgtatagcgatctggcaattgcgaatggtcgtggtc | 64 |
| L85D_p1 | gtaaccagaccacgaccattatcaattgccagatcgctatacacg | 65 |
| L85D_p2 | cgtgtatagcgatctggcaattgataatggtcgtggtctggttac | 66 |
| L85Q_p1 | cagaccacgaccattctgaattgccagatcgct | 67 |
| L85Q_p2 | agcgatctggcaattcagaatggtcgtggtctg | 68 |
| N86A_p1 | gtaaccagaccacgaccagccagaattgccagatcgcta | 69 |
| N86A_p2 | tagcgatctggcaattctggctggtcgtggtctggttac | 70 |
| N86C_p1 | gtaaccagaccacgaccacacagaattgccagatcgcta | 71 |
| N86C_p2 | tagcgatctggcaattctgtgtggtcgtggtctggttac | 72 |
| N86D_p1 | accagaccacgaccatccagaattgccagatcg | 73 |
| N86D_p2 | cgatctggcaattctggatggtcgtggtctggt | 74 |
| N86E_p1 | gtaaccagaccacgaccctccagaattgccagatcgc | 75 |
| N86E_p2 | gcgatctggcaattctggagggtcgtggtctggttac | 76 |
| N86F_p1 | gtaaccagaccacgaccaaacagaattgccagatcgcta | 77 |
| N86F_p2 | tagcgatctggcaattctgtttggtcgtggtctggttac | 78 |
| N86G_p1 | gtaaccagaccacgaccacccagaattgccagatcgcta | 79 |
| N86G_p2 | tagcgatctggcaattctgggtggtcgtggtctggttac | 80 |
| N86H_p1 | accagaccacgaccatgcagaattgccagatcg | 81 |
| N86H_p2 | cgatctggcaattctgcatggtcgtggtctggt | 82 |
| N86I_p1 | agaccacgaccaatcagaattgccagatcgctatac | 83 |
| N86I_p2 | gtatagcgatctggcaattctgattggtcgtggtct | 84 |
| N86K_p1 | aaccagaccacgacccttcagaattgccagatcg | 85 |
| N86K_p2 | cgatctggcaattctgaagggtcgtggtctggtt | 86 |
| N86L_p1 | gtggtaaccagaccacgacctagcagaattgccagatcgctat | 87 |
| N86L_p2 | atagcgatctggcaattctgctaggtcgtggtctggttaccac | 88 |
| N86M_p1 | ccagaccacgacccatcagaattgccagatcgctatacac | 89 |
| N86M_p2 | gtgtatagcgatctggcaattctgatgggtcgtggtctgg | 90 |
| N86P_p1 | gtaaccagaccacgaccaggcagaattgccagatcgcta | 91 |
| N86P_p2 | tagcgatctggcaattctgcctggtcgtggtctggttac | 92 |
| N86Q_p1 | gtaaccagaccacgaccctgcagaattgccagatcgc | 93 |
| N86Q_p2 | gcgatctggcaattctgcagggtcgtggtctggttac | 94 |
| N86R_p1 | ccagaccacgacccctcagaattgccagatcgctatacac | 95 |
| N86R_p2 | gtgtatagcgatctggcaattctgaggggtcgtggtctgg | 96 |
| N86S_p1 | agaccacgaccactcagaattgccagatcgctatac | 97 |
| N86S_p2 | gtatagcgatctggcaattctgagtggtcgtggtct | 98 |
| N86T_p1 | agaccacgaccagtcagaattgccagatcgctatac | 99 |
| N86T_p2 | gtatagcgatctggcaattctgactggtcgtggtct | 100 |
| N86V_p1 | gtaaccagaccacgaccaaccagaattgccagatcgcta | 101 |
| N86V_p2 | tagcgatctggcaattctggttggtcgtggtctggttac | 102 |
| N86W_p1 | gtggtaaccagaccacgaccccacagaattgccagatcgctat | 103 |
| N86W_p2 | atagcgatctggcaattctgtggggtcgtggtctggttaccac | 104 |
| N86Y_p1 | accagaccacgaccatacagaattgccagatcg | 105 |
| N86Y_p2 | cgatctggcaattctgtatggtcgtggtctggt | 106 |
| V91A_p1 | cggtactggtggtagccagaccacgacca | 107 |
| V91A_p2 | tggtcgtggtctggctaccaccagtaccg | 108 |
| V91D_p1 | cggtactggtggtatccagaccacgacca | 109 |
| V91D_p2 | tggtcgtggtctggataccaccagtaccg | 110 |
| V91G_p1 | cggtactggtggtacccagaccacgacca | 111 |
| V91G_p2 | tggtcgtggtctgggtaccaccagtaccg | 112 |
| V91L_p1 | cggtactggtggtaagcagaccacgaccatt | 113 |
| V91L_p2 | aatggtcgtggtctgcttaccaccagtaccg | 114 |
| V91Q_p1 | atctgcggtactggtggtctgcagaccacgaccattcag | 115 |
| V91Q_p2 | ctgaatggtcgtggtctgcagaccaccagtaccgcagat | 116 |
| V91S_p1 | ctgcggtactggtggtactcagaccacgaccattca | 117 |
| V91S_p2 | tgaatggtcgtggtctgagtaccaccagtaccgcag | 118 |
| V91T_p1 | ctgcggtactggtggtagtcagaccacgaccattca | 119 |
| V91T_p2 | tgaatggtcgtggtctgactaccaccagtaccgcag | 120 |
| T92A_p1 | cggtactggtggcaaccagaccacgacca | 121 |
| T92A_p2 | tggtcgtggtctggttgccaccagtaccg | 122 |
| T92D_p1 | gatctgcggtactggtgtcaaccagaccacgaccattc | 123 |
| T92D_p2 | gaatggtcgtggtctggttgacaccagtaccgcagatc | 124 |
| T92G_p1 | ctgcggtactggtgccaaccagaccacgacca | 125 |
| T92G_p2 | tggtcgtggtctggttggcaccagtaccgcag | 126 |
| T92L_p1 | tctgcggtactggttagaaccagaccacgaccattcagaattgc | 127 |
| T92L_p2 | gcaattctgaatggtcgtggtctggttctaaccagtaccgcaga | 128 |
| T92Q_p1 | tctgcggtactggtctgaaccagaccacgaccattcagaattgc | 129 |
| T92Q_p2 | gcaattctgaatggtcgtggtctggttcagaccagtaccgcaga | 130 |
| T92S_p1 | cggtactggtgctaaccagaccacgaccatt | 131 |
| T92S_p2 | aatggtcgtggtctggttagcaccagtaccg | 132 |
| L184A_p1 | atactctgaacataggccgccgcaacggtaaacggatg | 133 |
| L184A_p2 | catccgtttaccgttgcggcggcctatgttcagagtat | 134 |
| L184D_p1 | gatcatactctgaacataggcatccgcaacggtaaacggatgacg | 135 |
| L184D_p2 | cgtcatccgtttaccgttgcggatgcctatgttcagagtatgatc | 136 |
| L184Q_p1 | ctctgaacataggcctgcgcaacggtaaacg | 137 |
| L184Q_p2 | cgtttaccgttgcgcaggcctatgttcagag | 138 |
| Q188A_p1 | gtttcatgatcatactcgcaacataggccagcgcaacggt | 139 |
| Q188A_p2 | accgttgcgctggcctatgttgcgagtatgatcatgaaac | 140 |
| Q188D_p1 | cgtttcatgatcatactatcaacataggccagcgcaacggt | 141 |
| Q188D_p2 | accgttgcgctggcctatgttgatagtatgatcatgaaacg | 142 |
| Q188Q_p1 | gtttcatgatcatactttgaacataggccagcgcaac | 143 |
| Q188Q_p2 | gttgcgctggcctatgttcaaagtatgatcatgaaac | 144 |
| I191A_p1 | ggtgcttgcacgtttcatggccatactctgaacataggcc | 145 |
| I191A_p2 | ggcctatgttcagagtatggccatgaaacgtgcaagcacc | 146 |
| I191D_p1 | ggtgcttgcacgtttcatgtccatactctgaacataggcc | 147 |
| I191D_p2 | ggcctatgttcagagtatggacatgaaacgtgcaagcacc | 148 |
| I191Q_p1 | cagggtgcttgcacgtttcatctgcatactctgaacataggccag | 149 |
| I191Q_p2 | ctggcctatgttcagagtatgcagatgaaacgtgcaagcaccctg | 150 |
| I268A_p1 | acctgcgctcagaaaggtagcgatgttatcacgaatcagg | 151 |
| I268A_p2 | cctgattcgtgataacatcgctacctttctgagcgcaggt | 152 |
| I268D_p1 | acctgcgctcagaaaggtatcgatgttatcacgaatcagg | 153 |
| I268D_p2 | cctgattcgtgataacatcgatacctttctgagcgcaggt | 154 |
| I268Q_p1 | atgacctgcgctcagaaaggtctggatgttatcacgaatcaggct | 155 |
| I268Q_p2 | agcctgattcgtgataacatccagacctttctgagcgcaggtcat | 156 |
| T269A_p1 | cgctcagaaaggcaatgatgttatcacgaatcaggc | 157 |
| T269A_p2 | gcctgattcgtgataacatcattgcctttctgagcg | 158 |
| T269D_p1 | ttatgacctgcgctcagaaagtcaatgatgttatcacgaatcagg | 159 |
| T269D_p2 | cctgattcgtgataacatcattgactttctgagcgcaggtcataa | 160 |
| T269Q_p1 | gacctgcgctcagaaactgaatgatgttatcacgaatcaggctatcgt | 161 |
| T269Q_p2 | acgatagcctgattcgtgataacatcattcagtttctgagcgcaggtc | 162 |
| S272A_p1 | gaggtggtattatgacctgcggccagaaaggtaatgatgttatc | 163 |
| S272A_p2 | gataacatcattacctttctggccgcaggtcataataccacctc | 164 |
| S272C_p1 | gtggtattatgacctgcgcacagaaaggtaatgatgtta | 165 |
| S272C_p2 | taacatcattacctttctgtgcgcaggtcataataccac | 166 |
| S272D_p1 | gaggtggtattatgacctgcgtccagaaaggtaatgatgttatc | 167 |
| S272D_p2 | gataacatcattacctttctggacgcaggtcataataccacctc | 168 |
| S272E_p1 | gctgaggtggtattatgacctgcctccagaaaggtaatgatgttatcac | 169 |
| S272E_p2 | gtgataacatcattacctttctggaggcaggtcataataccacctcagc | 170 |
| S272F_p1 | gaggtggtattatgacctgcgaacagaaaggtaatgatgttatc | 171 |
| S272F_p2 | gataacatcattacctttctgttcgcaggtcataataccacctc | 172 |
| S272G_p1 | gtggtattatgacctgcgcccagaaaggtaatgatgtta | 173 |
| S272G_p2 | taacatcattacctttctgggcgcaggtcataataccac | 174 |
| S272H_p1 | gaggtggtattatgacctgcgtgcagaaaggtaatgatgttatc | 175 |
| S272H_p2 | gataacatcattacctttctgcacgcaggtcataataccacctc | 176 |
| S272I_p1 | gtattatgacctgcgatcagaaaggtaatgatgttatcacgaatc | 177 |
| S272I_p2 | gattcgtgataacatcattacctttctgatcgcaggtcataatac | 178 |
| S272K_p1 | ggtattatgacctgccttcagaaaggtaatgatgttatcacgaatc | 179 |
| S272K_p2 | gattcgtgataacatcattacctttctgaaggcaggtcataatacc | 180 |
| S272L_p1 | gctgaggtggtattatgacctgctagcagaaaggtaatgatgttatcac | 181 |
| S272L_p2 | gtgataacatcattacctttctgctagcaggtcataataccacctcagc | 182 |
| S272M_p1 | ggtattatgacctgccatcagaaaggtaatgatgttatcacgaatc | 183 |
| S272M_p2 | gattcgtgataacatcattacctttctgatggcaggtcataatacc | 184 |
| S272N_p1 | gtattatgacctgcgttcagaaaggtaatgatgttatcacgaatc | 185 |
| S272N_p2 | gattcgtgataacatcattacctttctgaacgcaggtcataatac | 186 |
| S272P_p1 | gaggtggtattatgacctgcgggcagaaaggtaatgatgttatc | 187 |
| S272P_p2 | gataacatcattacctttctgcccgcaggtcataataccacctc | 188 |
| S272Q_p1 | gctgaggtggtattatgacctgcctgcagaaaggtaatgatgttatcac | 189 |
| S272Q_p2 | gtgataacatcattacctttctgcaggcaggtcataataccacctcagc | 190 |
| S272R_p1 | ggtggtattatgacctgccctcagaaaggtaatgatg | 191 |
| S272R_p2 | catcattacctttctgagggcaggtcataataccacc | 192 |
| S272T_p1 | attatgacctgcggtcagaaaggtaatgatgttatcacg | 193 |
| S272T_p2 | cgtgataacatcattacctttctgaccgcaggtcataat | 194 |
| S272V_p1 | gaggtggtattatgacctgcgaccagaaaggtaatgatgttatc | 195 |
| S272V_p2 | gataacatcattacctttctggtcgcaggtcataataccacctc | 196 |
| S272W_p1 | gaggtggtattatgacctgcccacagaaaggtaatgatgttat | 197 |
| S272W_p2 | ataacatcattacctttctgtgggcaggtcataataccacctc | 198 |
| S272Y_p1 | gctgaggtggtattatgacctgcatacagaaaggtaatgatgttatcac | 199 |
| S272Y_p2 | gtgataacatcattacctttctgtatgcaggtcataataccacctcagc | 200 |
| A273D_p1 | gctgaggtggtattatgaccatcgctcagaaaggtaatgatg | 201 |
| A273D_p2 | catcattacctttctgagcgatggtcataataccacctcagc | 202 |
| A273G_p1 | aggtggtattatgacctccgctcagaaaggtaatg | 203 |
| A273G_p2 | cattacctttctgagcggaggtcataataccacct | 204 |
| A273Q_p1 | aaatgctgaggtggtattatgaccctggctcagaaaggtaatgatgttatc | 205 |
| A273Q_p2 | gataacatcattacctttctgagccagggtcataataccacctcagcattt | 206 |
| N276A_p1 | atgctgaggtggtagcatgacctgcgctcagaaaggtaatg | 207 |
| N276A_p2 | cattacctttctgagcgcaggtcatgctaccacctcagcat | 208 |
| N276C_p1 | atgctgaggtggtacaatgacctgcgctcagaaaggtaatg | 209 |
| N276C_p2 | cattacctttctgagcgcaggtcattgtaccacctcagcat | 210 |
| N276D_p1 | aaatgctgaggtggtatcatgacctgcgctcagaa | 211 |
| N276D_p2 | ttctgagcgcaggtcatgataccacctcagcattt | 212 |
| N276E_p1 | gaaatgctgaggtggtctcatgacctgcgctcagaaagg | 213 |
| N276E_p2 | cctttctgagcgcaggtcatgagaccacctcagcatttc | 214 |
| N276F_p1 | atgctgaggtggtaaaatgacctgcgctcagaaaggtaatg | 215 |
| N276F_p2 | cattacctttctgagcgcaggtcattttaccacctcagcat | 216 |
| N276G_p1 | atgctgaggtggtaccatgacctgcgctcagaaaggtaatg | 217 |
| N276G_p2 | cattacctttctgagcgcaggtcatggtaccacctcagcat | 218 |
| N276H_p1 | aaatgctgaggtggtatgatgacctgcgctcagaa | 219 |
| N276H_p2 | ttctgagcgcaggtcatcataccacctcagcattt | 220 |
| N276I_p1 | aaatgctgaggtggtaatatgacctgcgctcagaa | 221 |
| N276I_p2 | ttctgagcgcaggtcatattaccacctcagcattt | 222 |
| N276K_p1 | atgctgaggtggtcttatgacctgcgctcagaaag | 223 |
| N276K_p2 | ctttctgagcgcaggtcataagaccacctcagcat | 224 |
| N276L_p1 | tcagaaatgctgaggtggttagatgacctgcgctcagaaaggtaa | 225 |
| N276L_p2 | ttacctttctgagcgcaggtcatctaaccacctcagcatttctga | 226 |
| N276M_p1 | atgctgaggtggtcatatgacctgcgctcagaaaggtaat | 227 |
| N276M_p2 | attacctttctgagcgcaggtcatatgaccacctcagcat | 228 |
| N276P_p1 | atgctgaggtggtaggatgacctgcgctcagaaaggtaatg | 229 |
| N276P_p2 | cattacctttctgagcgcaggtcatcctaccacctcagcat | 230 |
| N276Q_p1 | gaaatgctgaggtggtctgatgacctgcgctcagaaagg | 231 |
| N276Q_p2 | cctttctgagcgcaggtcatcagaccacctcagcatttc | 232 |
| N276R_p1 | atgctgaggtggtcctatgacctgcgctcagaaaggtaat | 233 |
| N276R_p2 | attacctttctgagcgcaggtcataggaccacctcagcat | 234 |
| N276S_p1 | aaatgctgaggtggtactatgacctgcgctcagaa | 235 |
| N276S_p2 | ttctgagcgcaggtcatagtaccacctcagcattt | 236 |
| N276T_p1 | aaatgctgaggtggtagtatgacctgcgctcagaa | 237 |
| N276T_p2 | ttctgagcgcaggtcatactaccacctcagcattt | 238 |
| N276V_p1 | atgctgaggtggtaacatgacctgcgctcagaaaggtaatg | 239 |
| N276V_p2 | cattacctttctgagcgcaggtcatgttaccacctcagcat | 240 |
| N276W_p1 | tcagaaatgctgaggtggtccaatgacctgcgctcagaaaggtaa | 241 |
| N276W_p2 | ttacctttctgagcgcaggtcattggaccacctcagcatttctga | 242 |
| N276Y_p1 | aaatgctgaggtggtataatgacctgcgctcagaa | 243 |
| N276Y_p2 | ttctgagcgcaggtcattataccacctcagcattt | 244 |
| T277A_p1 | atgctgaggtggcattatgacctgcgctcagaaagg | 245 |
| T277A_p2 | cctttctgagcgcaggtcataatgccacctcagcat | 246 |
| T277D_p1 | cagaaatgctgaggtgtcattatgacctgcgctcagaaagg | 247 |
| T277D_p2 | cctttctgagcgcaggtcataatgacacctcagcatttctg | 248 |
| T277Q_p1 | cagaaatgctgaggtctgattatgacctgcgctcagaaaggtaatga | 249 |
| T277Q_p2 | tcattacctttctgagcgcaggtcataatcagacctcagcatttctg | 250 |
| I339A_p1 | gtatttcagaatgctggtagcaggcggatgaatacgcagg | 251 |
| I339A_p2 | cctgcgtattcatccgcctgctaccagcattctgaaatac | 252 |
| I339D_p1 | gtatttcagaatgctggtatcaggcggatgaatacgcagg | 253 |
| I339D_p2 | cctgcgtattcatccgcctgataccagcattctgaaatac | 254 |
| I339Q_p1 | atttcagaatgctggtctgaggcggatgaatacgcaggctttctttga | 255 |
| I339Q_p2 | tcaaagaaagcctgcgtattcatccgcctcagaccagcattctgaaat | 256 |
| S341A_p1 | tcttttttacagtatttcagaatggcggtaataggcggatgaatacgca | 257 |
| S341A_p2 | tgcgtattcatccgcctattaccgccattctgaaatactgtaaaaaaga | 258 |
| S341C_p1 | ttttacagtatttcagaatgcaggtaataggcggatgaatacg | 259 |
| S341C_p2 | cgtattcatccgcctattacctgcattctgaaatactgtaaaa | 260 |
| S341D_p1 | tcttttttacagtatttcagaatgtcggtaataggcggatgaatacgcag | 261 |
| S341D_p2 | ctgcgtattcatccgcctattaccgacattctgaaatactgtaaaaaaga | 262 |
| S341E_p1 | cgtcttttttacagtatttcagaatctcggtaataggcggatgaatacgcagg | 263 |
| S341E_p2 | cctgcgtattcatccgcctattaccgagattctgaaatactgtaaaaaagacg | 264 |
| S341F_p1 | tcttttttacagtatttcagaatgaaggtaataggcggatgaatacgcag | 265 |
| S341F_p2 | ctgcgtattcatccgcctattaccttcattctgaaatactgtaaaaaaga | 266 |
| S341G_p1 | ttttacagtatttcagaatgccggtaataggcggatgaatacg | 267 |
| S341G_p2 | cgtattcatccgcctattaccggcattctgaaatactgtaaaa | 268 |
| S341H_p1 | tcttttttacagtatttcagaatgtgggtaataggcggatgaatacgcag | 269 |
| S341H_p2 | ctgcgtattcatccgcctattacccacattctgaaatactgtaaaaaaga | 270 |
| S341I_p1 | cttttttacagtatttcagaatgatggtaataggcggatgaatacgc | 271 |
| S341I_p2 | gcgtattcatccgcctattaccatcattctgaaatactgtaaaaaag | 272 |
| S341K_p1 | gtcttttttacagtatttcagaatcttggtaataggcggatgaatacgc | 273 |
| S341K_p2 | gcgtattcatccgcctattaccaagattctgaaatactgtaaaaaagac | 274 |
| S341L_p1 | cgtcttttttacagtatttcagaattagggtaataggcggatgaatacgcagg | 275 |
| S341L_p2 | cctgcgtattcatccgcctattaccctaattctgaaatactgtaaaaaagacg | 276 |
| S341M_p1 | gtcttttttacagtatttcagaatcatggtaataggcggatgaatacgc | 277 |
| S341M_p2 | gcgtattcatccgcctattaccatgattctgaaatactgtaaaaaagac | 278 |
| S341N_p1 | cttttttacagtatttcagaatgttggtaataggcggatgaatacgc | 279 |
| S341N_p2 | gcgtattcatccgcctattaccaacattctgaaatactgtaaaaaag | 280 |
| S341P_p1 | tcttttttacagtatttcagaatgggggtaataggcggatgaatacgca | 281 |
| S341P_p2 | tgcgtattcatccgcctattacccccattctgaaatactgtaaaaaaga | 282 |
| S341Q_p1 | cgtcttttttacagtatttcagaatctgggtaataggcggatgaatacgcagg | 283 |
| S341Q_p2 | cctgcgtattcatccgcctattacccagattctgaaatactgtaaaaaagacg | 284 |
| S341R_p1 | tcttttttacagtatttcagaatcctggtaataggcggatgaatac | 285 |
| S341R_p2 | gtattcatccgcctattaccaggattctgaaatactgtaaaaaaga | 286 |
| S341T_p1 | ttttttacagtatttcagaatggtggtaataggcggatgaatacg | 287 |
| S341T_p2 | cgtattcatccgcctattaccaccattctgaaatactgtaaaaaa | 288 |
| S341V_p1 | tcttttttacagtatttcagaatgacggtaataggcggatgaatacgcag | 289 |
| S341V_p2 | ctgcgtattcatccgcctattaccgtcattctgaaatactgtaaaaaaga | 290 |
| S341W_p1 | tcttttttacagtatttcagaatccaggtaataggcggatgaatacgc | 291 |
| S341W_p2 | gcgtattcatccgcctattacctggattctgaaatactgtaaaaaaga | 292 |
| S341Y_p1 | cgtcttttttacagtatttcagaatataggtaataggcggatgaatacgcagg | 293 |
| S341Y_p2 | cctgcgtattcatccgcctattacctatattctgaaatactgtaaaaaagacg | 294 |
| I342A_p1 | cgtcttttttacagtatttcagagcgctggtaataggcggatgaatac | 295 |
| I342A_p2 | gtattcatccgcctattaccagcgctctgaaatactgtaaaaaagacg | 296 |
| I342D_p1 | cgtcttttttacagtatttcagatcgctggtaataggcggatgaatac | 297 |
| I342D_p2 | gtattcatccgcctattaccagcgatctgaaatactgtaaaaaagacg | 298 |
| I342Q_p1 | tgcgtcttttttacagtatttcagctggctggtaataggcggatgaatacg | 299 |
| I342Q_p2 | cgtattcatccgcctattaccagccagctgaaatactgtaaaaaagacgca | 300 |
| I342V_p1 | gtcttttttacagtatttcagaacgctggtaataggcggatgaata | 301 |
| I342V_p2 | tattcatccgcctattaccagcgttctgaaatactgtaaaaaagac | 302 |
| V452A_p1 | caaccggtttggtgctaacagcaactgcataaccaattttc | 303 |
| V452A_p2 | gaaaattggttatgcagttgctgttagcaccaaaccggttg | 304 |
| V452D_p1 | caaccggtttggtgctaacatcaactgcataaccaattttc | 305 |
| V452D_p2 | gaaaattggttatgcagttgatgttagcaccaaaccggttg | 306 |
| V452I_p1 | tttggtgctaacaataactgcataaccaattttctggctcg | 307 |
| V452I_p2 | cgagccagaaaattggttatgcagttattgttagcaccaaa | 308 |
| V452Q_p1 | aaccaaccggtttggtgctaacctgaactgcataaccaattttctgg | 309 |
| V452Q_p2 | ccagaaaattggttatgcagttcaggttagcaccaaaccggttggtt | 310 |
| V453A_p1 | caaccggtttggtgctagcaacaactgcataacca | 311 |
| V453A_p2 | tggttatgcagttgttgctagcaccaaaccggttg | 312 |
| V453D_p1 | caaccggtttggtgctatcaacaactgcataacca | 313 |
| V453D_p2 | tggttatgcagttgttgatagcaccaaaccggttg | 314 |
| V453I_p1 | ccaaccggtttggtgctaataacaactgcataaccaatt | 315 |
| V453I_p2 | aattggttatgcagttgttattagcaccaaaccggttgg | 316 |
| V453Q_p1 | aaccggtttggtgctctgaacaactgcataaccaattttctggctcg | 317 |
| V453Q_p2 | cgagccagaaaattggttatgcagttgttcagagcaccaaaccggtt | 318 |

### Example 3: Cell Cultures and Product Analysis

Wild type or mutant plasmids were transferred into BL21(DE3) cells and were cultured overnight at a temperature of 37° C. On the morning of the following day, the overnight cultures were diluted at a ratio of 1:100 into 5 ml of LB medium and were cultured at 37° C. When the OD₆₀₀ reached a value of 1.2, isopropyl β-d-1-thiogalactopyranoside (IPTG) was added at a concentration 1.0 mM to induce expression of the wild-type MaP450 enzyme and the mutant enzymes in the different cell culture samples, respectively. Following overnight incubation at 16° C., cells were collected and re-suspended in a total solution of 0.5 ml, of a buffer containing 20 mM of Tris (pH 7.0) and 1 mM of NADPH, at a cell concentration of 50 g/L fresh weight in BD round tubes (14 ml). 2.0 g/L of lauric acid was added as the substrate and then the mixture was incubated at 30° C. and shaken at 150 rpm for 2 hours.

To compare the selectivity of the various mutants, GC/MS and GC/FID analyses were performed to analyze the distribution of the resulting hydroxylated fatty acid and lactone products. Specifically, 500 μl of each culture was transferred to 1.5 Eppendorf tubes and mixed with 500 μL ethyl acetate and 2 μL of 2N HCl. The acidified culture was extracted with 0.5 ml ethyl acetate by shaking at room temperature for 30 min. After centrifugation at 14,000 g for 15 minutes, the ethyl acetate phase was subjected to GC/MS and GC/FID analysis.

GC/MS analysis was carried out on a Shimadzu GC-2010 system coupled with a GCMS-QP2010S detector. The analytical column was a SHRXI-5MS (thickness 0.25 μm; length 30 m; diameter 0.25 mm) and the injection temperature was 265° C. under split mode. The temperature gradient was from 0 to 3 min at 80° C.; 3-8.7 min from 120° C. to 263° C., at a temperature gradient of about 25° C. per minute, then from 8.7 to 10.7 min at 263° C.

GC/FID analysis was conducted on Shimadzu GC-2014 system. The analytical column was Restek RXi-5 ms (thickness 0.25 μm; length 30 m; diameter 0.25 mm) and the injection temperature was 240° C. under split mode. The temperature gradient was 0 to 3 min at 100° C.; from 3 to 9 min at 100° C. to 280° C., at gradient of 30° C. per minute, then from 9 to 12 min at 280° C.

As illustrated in the GC/MS chromatograms in FIG. 2, while the wild-type MaP450 enzyme has a 98.1:1.9 formation ratio of γ-dodecalactone to δ-dodecalactone, out of the various mutants that were investigated, the mutant enzyme with the best selectivity, namely S272N (SEQ ID NO: 3), unexpectedly yielded a 23.4:76.6 formation ratio of γ-dodecalactone to δ-dodecalactone. In other words, the S272N mutant enzyme was able to improve the formation rate of δ-dodecalactone by a surprising 21 times as compared with the wild-type enzyme.

Several potential single mutants making higher δ-Dodecalactone were confirmed by GC-MS (Table 2). The mutants were identified (such as S272I, S272L, S272M, S272N, S272T and N276T) that produce significantly higher amount of δ-Dodecalactone, as compared to that of wild type (Table 2). Mutants S272T produced 33.7 times, and mutant N276T produced 17.7 times more δ-Dodecalactone, as compared to that of wild type.

TABLE 2
γ-Dodecalactone and δ-Dodecalactone formation
rates from lauric acid for pET16b expressing single
mutants of MaP450 as determined by GC-MS & GC-FID

| | C4—OH LA | C5—OH LA | | wild | |
|---|---|---|---|---|---|
| | (nmol/min/mg | (nmol/min/mg | C5/(C5 + | type | mutant |
| mutant | cells) | cells) | C4) (%) | DNA | DNA |
| wild type | 2.570 | 0.049 | 1.9 | AAA | |
| K73A | 0.011 | 0.003 | 21.4 | AAA | GCA |
| K73C | 0.072 | 0.000 | 0.0 | | TGC |
| K73G | 0.009 | 0.000 | 0.0 | | GGA |
| K73N | 0.038 | 0.000 | 0.0 | | AAT |
| K73R | 0.862 | 0.022 | 2.4 | | AGA |
| K73Y | 0.010 | 0.007 | 41.2 | | TAT |
| Y79A | 0.184 | 0.016 | 7.8 | TAT | GCT |
| Y79C | 0.352 | 0.005 | 1.3 | | TGT |
| Y79K | 0.031 | 0.004 | 11.4 | | AAG |
| Y79N | 0.454 | 0.031 | 6.4 | | AAT |
| Y79R | 0.012 | 0.003 | 20.7 | | CGT |
| Y79S | 0.287 | 0.005 | 1.5 | | AGT |
| L82A | 0.356 | 0.054 | 13.1 | CTG | GCG |
| L85A | 1.271 | 0.074 | 5.5 | CTG | GCG |
| N86A | 1.002 | 0.088 | 8.1 | AAT | GCT |
| V91A | 0.804 | 0.083 | 9.4 | GTT | GCT |
| T92A | 0.356 | 0.018 | 4.8 | ACC | GCC |
| L184A | 0.169 | 0.008 | 4.2 | CTG | GCG |
| Q188A | 0.365 | 0.035 | 8.6 | CAA | GCG |
| I191A | 1.301 | 0.068 | 5.0 | ATC | GCC |
| I268A | 0.522 | 0.026 | 4.7 | ATT | GCT |
| T269A | 0.658 | 0.028 | 4.1 | ACC | GCC |
| S272A | 0.524 | 0.285 | 35.2 | AGC | GCC |
| S272C | 0.767 | 0.156 | 16.9 | | TGC |
| S272D | 0.104 | 0.105 | 50.2 | | GAC |
| S272E | 0.438 | 0.158 | 26.5 | | GAG |
| S272F | 0.043 | 0.050 | 53.8 | | TTC |
| S272G | 1.065 | 0.858 | 44.6 | | GGC |
| S272H | 0.684 | 0.095 | 12.2 | | CAC |
| S272I | 0.746 | 1.293 | 63.4 | | ATC |
| S272K | 0.163 | 0.091 | 35.8 | | AAG |
| S272L | 0.490 | 1.253 | 71.9 | | CTA |
| S272M | 0.738 | 1.004 | 57.6 | | ATG |
| S272N | 0.314 | 1.029 | 76.6 | | AAC |
| S272P | 0.248 | 0.149 | 37.5 | | CCC |
| S272Q | 0.798 | 0.449 | 36.0 | | CAG |
| S272T | 0.931 | 1.653 | 64.0 | | ACC |
| S272V | 0.924 | 0.968 | 51.2 | | GTC |
| S272W | 0.000 | 0.113 | 100.0 | | TGG |
| S272Y | 0.000 | 0.021 | 100.0 | | TAT |
| A273G | 0.339 | 0.016 | 4.5 | GCA | GGA |
| N276A | 0.324 | 0.196 | 37.6 | AAT | GCT |
| N276D | 0.000 | 0.000 | 0.0 | | GAT |
| N276E | 0.273 | 0.136 | 33.3 | | GAG |
| N276I | 0.550 | 0.335 | 37.9 | | ATT |
| N276K | 0.166 | 0.154 | 48.1 | | AAG |
| N276L | 0.229 | 0.208 | 47.6 | | CTA |
| N276M | 0.589 | 0.293 | 33.2 | | ATG |
| N276P | 0.117 | 0.042 | 26.4 | | CCT |
| N276Q | 0.911 | 0.291 | 24.2 | | CAG |
| N276R | 0.204 | 0.062 | 23.3 | | AGG |
| N276T | 1.569 | 0.871 | 35.7 | | ACT |
| N276V | 0.725 | 0.320 | 30.6 | | GTT |
| N276W | 0.109 | 0.057 | 34.3 | | TGG |
| N276Y | 0.026 | 0.027 | 50.9 | | TAT |
| T277A | 0.234 | 0.021 | 8.1 | ACC | GCC |
| I339A | 1.916 | 0.254 | 11.7 | ATT | GCT |
| S341A | 2.187 | 0.111 | 4.8 | AGC | GCC |
| I342A | 2.076 | 0.068 | 3.2 | ATT | GCT |
| V452A | 0.765 | 0.061 | 7.4 | GTT | GCT |
| V453A | 0.789 | 0.069 | 8.0 | GTT | GCT |

Several potential double mutants showing higher activity are confirmed by GC-MS (Table 3). Mutagenesis at N86 and 341 may further increase the production of δ-Dodecalactone when single mutation S272N and S272T is as the first mutant site (FIG. 6). Double mutants, S272N/N86E, S272N/N86M, S272N/S341G, S272N/S341H, S272N/S341N, S272T/N86F, S272T/N86I, S272T/N86V and S272T/N86W produced 30 times more δ-Dodecalactone, as compared to that of wild type.

TABLE 3
γ-Dodecalactone and δ-Dodecalactone formation rates from lauric acid for pET16b
expressing double mutants of MaP450 as determined by GC-MS & GC-FID

| | C4-OH LA | C5-OH LA | | | |
|---|---|---|---|---|---|
| | (nmol/min/mg | (nmol/min/mg | C5/(C5 + C4) | wild type | mutant |
| mutant | cells) | cells) | (%) | DNA | DNA |
| S272A/K73G | 0.000 | 0.000 | 0.0 | AGC/AAA | GCC/GGA |
| S272A/K73L | 0.076 | 0.023 | 23.2 | | GCC/TTA |
| S272A/K73S | 0.000 | 0.000 | 0.0 | | GCC/AGC |
| S272A/K73T | 0.021 | 0.001 | 4.5 | | GCC/ACG |
| S272A/Y79G | 0.037 | 0.028 | 43.1 | AGC/TAT | GCC/GGT |
| S272A/Y79D | 0.055 | 0.028 | 33.7 | | GCC/GAT |
| S272A/Y79L | 1.291 | 0.664 | 34.0 | | GCC/TTA |
| S272A/Y79N | 0.315 | 0.151 | 32.4 | | GCC/AAT |
| S272A/Y79T | 0.077 | 0.037 | 32.5 | | GCC/ACT |
| S272A/L82A | 0.108 | 0.053 | 32.9 | AGC/CTG | GCC/GCG |
| S272A/L82Q | 0.270 | 0.191 | 41.4 | | GCC/CAG |
| S272A/L85A | 0.646 | 0.347 | 34.9 | AGC/CTG | GCC/GCG |
| S272A/L85D | 0.000 | 0.019 | 100.0 | | GCC/GAT |
| S272A/L85Q | 0.411 | 0.270 | 39.6 | | GCC/CAG |
| S272A/N86D | 0.786 | 1.401 | 64.1 | AGC/AAT | AAC/GAT |
| S272A/N86G | 0.886 | 0.374 | 29.7 | | AAC/GGT |
| S272A/V91A | 0.249 | 0.212 | 46.0 | AGC/GTT | GCC/GCT |
| S272A/V91D | 0.044 | 0.046 | 51.1 | | GCC/GAT |
| S272A/V91G | 0.067 | 0.028 | 29.5 | | GCC/GGT |
| S272A/V91L | 0.785 | 0.257 | 24.7 | | GCC/CTT |
| S272A/V91Q | 0.090 | 0.073 | 44.8 | | GCC/CAG |
| S272A/V91S | 0.134 | 0.124 | 48.1 | | GCC/AGT |
| S272A/V91T | 0.216 | 0.096 | 30.8 | | GCC/ACT |
| S272A/T92A | 0.000 | 0.000 | 0.0 | AGC/ACC | GCC/GCC |
| S272A/T92D | 0.030 | 0.046 | 60.5 | | GCC/GAC |
| S272A/T92G | 0.105 | 0.037 | 26.1 | | GCC/GGC |
| S272A/T92L | 0.056 | 0.031 | 35.6 | | GCC/CTA |
| S272A/T92Q | 0.000 | 0.012 | 100.0 | | GCC/CAG |
| S272A/T92S | 0.860 | 0.406 | 32.1 | | GCC/GGC |
| S272A/Q188A | 0.120 | 0.031 | 20.5 | AGC/CAA | GCC/GCG |
| S272A/I191A | 0.962 | 0.448 | 31.8 | AGC/ATC | GCC/GCC |
| S272A/N276S | 0.372 | 0.956 | 72.0 | AGC/AAT | GCC/AGT |
| S272A/I339A | 0.056 | 0.018 | 24.3 | AGC/ATT | GCC/GCT |
| S272A/S341G | 1.104 | 0.871 | 44.1 | AGC/AGC | GCC/GGC |
| S272A/V453I | 1.000 | 0.780 | 43.8 | AGC/GTT | GCC/ATT |
| S272N/N86A | 0.821 | 1.439 | 63.7 | | AAC/GCT |
| S272N/N86C | 0.287 | 0.930 | 76.4 | | AAC/TGT |
| S272N/N86E | 0.412 | 1.702 | 80.5 | | AAC/GAG |
| S272N/N86F | 0.043 | 0.048 | 52.7 | | AAC/TTT |
| S272N/N86H | 0.400 | 0.936 | 70.1 | | AAC/CAT |
| S272N/N86I | 0.244 | 0.575 | 70.2 | | AAC/ATT |
| S272N/N86L | 0.650 | 0.930 | 58.9 | | AAC/CTA |
| S272N/N86M | 0.280 | 2.581 | 90.2 | | AAC/ATG |
| S272N/N86P | 0.038 | 0.073 | 65.8 | | AAC/CCT |
| S272N/N86Q | 1.161 | 0.267 | 18.7 | | AAC/CAG |
| S272N/N86R | 0.019 | 0.018 | 48.6 | | AAC/AGG |
| S272N/N86S | 0.540 | 0.830 | 60.6 | | AAC/AGT |
| S272N/N86T | 0.389 | 0.689 | 63.9 | | AAC/ACT |
| S272N/N86V | 0.182 | 0.344 | 65.4 | | AAC/GTT |
| S272N/N86W | 0.046 | 0.221 | 82.8 | | AAC/TGG |
| S272N/N86Y | 0.048 | 0.050 | 51.0 | | AAC/TAT |
| S272N/S341A | 0.000 | 0.000 | 0.0 | AGC/AGC | AAC/GCC |
| S272N/S341C | 0.406 | 1.634 | 80.1 | | AAC/TGC |
| S272N/S341D | 0.338 | 1.428 | 80.9 | | AAC/GAC |
| S272N/S341E | 0.302 | 1.505 | 83.3 | | AAC/GAG |
| S272N/S341F | 0.359 | 1.523 | 80.9 | | AAC/TTC |
| S272N/S341G | 0.408 | 1.766 | 81.2 | | AAC/GGC |
| S272N/S341H | 0.360 | 1.519 | 80.8 | | AAC/CAC |
| S272N/S341I | 0.288 | 1.130 | 79.7 | | AAC/ATC |
| S272N/S341K | 0.135 | 0.734 | 84.5 | | AAC/AAG |
| S272N/S341L | 0.411 | 1.267 | 75.5 | | AAC/CTA |
| S272N/S341M | 0.359 | 1.420 | 79.8 | | AAC/ATG |
| S272N/S341N | 0.341 | 1.527 | 81.8 | | AAC/AAC |
| S272N/S341P | 0.053 | 0.107 | 66.9 | | AAC/CCC |
| S272N/S341Q | 0.400 | 1.502 | 79.0 | | AAC/CAG |
| S272N/S341R | 0.070 | 0.232 | 76.9 | | AAC/AGG |
| S272N/S341T | 0.337 | 1.269 | 79.0 | | AAC/ACC |
| S272N/S341V | 0.330 | 1.304 | 79.8 | | AAC/GTC |
| S272N/S341Y | 0.420 | 1.427 | 77.3 | | AAC/TAT |
| S272T/N86C | 0.231 | 0.710 | 75.4 | AGC/AAT | ACC/TGT |
| S272T/N86D | 0.362 | 1.117 | 75.5 | | ACC/GAT |
| S272T/N86E | 0.951 | 1.192 | 55.6 | | ACC/GAG |
| S272T/N86F | 0.088 | 2.028 | 95.8 | | ACC/TTT |
| S272T/N86G | 1.009 | 0.886 | 46.8 | | ACC/GGT |
| S272T/N86H | 0.584 | 1.262 | 68.4 | | ACC/CAT |
| S272T/N86I | 0.322 | 1.896 | 85.5 | | ACC/ATT |
| S272T/N86K | 0.257 | 1.128 | 81.4 | | ACC/AAG |
| S272T/N86L | 0.201 | 1.770 | 89.8 | | ACC/CTA |
| S272T/N86M | 0.119 | 1.448 | 92.4 | | ACC/ATG |
| S272T/N86P | 0.056 | 0.111 | 66.6 | | ACC/CCT |
| S272T/N86Q | 0.776 | 0.84 | 52.0 | | ACC/CAG |
| S272T/N86R | 0.403 | 0.056 | 12.2 | | ACC/AGG |
| S272T/N86S | 0.943 | 1.201 | 56.0 | | ACC/AGT |
| S272T/N86T | 0.508 | 1.399 | 73.4 | | ACC/ACT |
| S272T/N86V | 0.201 | 1.464 | 87.9 | | ACC/GTT |
| S272T/N86W | 0.201 | 1.464 | 87.9 | | ACC/TGG |
| S272T/N86Y | 0.217 | 0.522 | 70.6 | | ACC/TAT |
| S272T/S341A | 0.861 | 1.878 | 68.6 | AGC/AGC | ACC/GCC |
| S272T/S341C | 0.603 | 1.335 | 68.9 | | ACC/TGC |
| S272T/S341D | 0.473 | 1.158 | 71.0 | | ACC/GAC |
| S272T/S341E | 0.415 | 1.41 | 77.3 | | ACC/GAG |
| S272T/S341F | 0.716 | 1.678 | 70.1 | | ACC/TTC |
| S272T/S341G | 0.563 | 1.567 | 73.6 | | ACC/GGC |
| S272T/S341H | 0.552 | 1.459 | 72.6 | | ACC/CAC |
| S272T/S341I | 0.486 | 1.13 | 69.9 | | ACC/ATC |
| S272T/S341K | 0.286 | 1.145 | 80.0 | | ACC/AAG |
| S272T/S341L | 1.048 | 1.523 | 59.2 | | ACC/CTA |
| S272T/S341M | 0.795 | 1.525 | 65.7 | | ACC/ATG |
| S272T/S341N | 0.474 | 1.559 | 76.7 | | ACC/AAC |
| S272T/S341P | 0.835 | 0.873 | 51.1 | | ACC/CCC |
| S272T/S341Q | 0.454 | 1.138 | 71.5 | | ACC/CAG |
| S272T/S341R | 0 | 0.045 | 100.0 | | ACC/AGG |
| S272T/S341T | 0.473 | 1.129 | 70.5 | | ACC/ACC |
| S272T/S341V | 0.55 | 1.496 | 73.1 | | ACC/GTC |
| S272T/S341W | 0.8 | 1.338 | 62.6 | | ACC/TGG |
| S272T/S341Y | 0.556 | 1.258 | 69.3 | | ACC/TAT |
| N276A/K73A | 0.000 | 0.005 | 100.0 | AAT/AAA | GCT/GCA |
| N276A/K73C | 0.000 | 0.000 | 0.0 | | GCT/TGC |
| N276A/K73G | 0.000 | 0.000 | 0.0 | | GCT/GGA |
| N276A/K73L | 0.000 | 0.000 | 0.0 | | GCT/TTA |
| N276A/K73N | 0.000 | 0.000 | 0.0 | | GCT/AAT |
| N276A/K73R | 0.000 | 0.000 | 0.0 | | GCT/AGA |
| N276A/K73S | 0.000 | 0.011 | 100.0 | | GCT/AGC |
| N276A/K73T | 0.000 | 0.000 | 0.0 | | GCT/ACG |
| N276A/K73Y | 0.000 | 0.000 | 0.0 | | GCT/TAT |
| N276A/Y79A | 0.021 | 0.000 | 0.0 | AAT/TAT | GCT/GCT |
| N276A/Y79C | 0.000 | 0.000 | 0.0 | | GCT/TGT |
| N276A/Y79G | 0.000 | 0.000 | 0.0 | | GCT/GGT |
| N276A/Y79N | 0.000 | 0.000 | 0.0 | | GCT/AAT |
| N276A/Y79S | 0.000 | 0.000 | 0.0 | | GCT/AGT |
| N276A/Y79T | 0.000 | 0.000 | 0.0 | | GCT/ACT |
| N276A/L82A | 0.000 | 0.007 | 100.0 | AAT/CTG | GCT/GCG |
| N276A/N86L | 0.036 | 0.042 | 53.8 | AAT/AAT | GCT/CTA |
| N276A/N86S | 0.119 | 0.071 | 37.4 | | GCT/AGT |
| N276A/N86T | 0.000 | 0.000 | 0.0 | | GCT/ACT |
| N276A/Q188A | 0.000 | 0.000 | 0.0 | AAT/CAA | GCT/GCG |
| N276A/S272A | 1.348 | 0.765 | 36.2 | AAT/AGC | GCT/GCC |
| N276A/1339A | 0.046 | 0.033 | 41.8 | AAT/ATT | GCT/GCT |

The triple mutant, S272N/N86M/S341D showing the highest reaction rate making δ-Dodecalactone was confirmed by GC-MS and produced 61.5 times more δ-Dodecalactone, as compared to that of wild type (FIG. 7) and another triple mutant, S272T/N86F/S341H could make highest percentage of δ-Dodecalactone and the ratio of γ-Dodecalactone and δ-Dodecalactone was 98.1:1.9, which also produced 59 times more δ-Dodecalactone, as compared to that of wild type (FIG. 7, Table 4). Triple mutants, S272N/N86M/S341D, S272N/N86M/S341H, S272T/N86F/S341A, S272T/N86F/S341C, S272T/N86F/S341H, S272T/N86F/S341M, S272T/N86F/S341Q and S272T/N86F/S341T produced 40 times more δ-Dodecalactone, as compared to that of wild type.

TABLE 4
γ-Dodecalactone and δ-Dodecalactone formation rates from lauric acid for pET16b
expressing triple mutants of MaP450 as determined by GC-MS & GC-FID

| | C4-OH LA | C5-OH LA | | | |
|---|---|---|---|---|---|
| | (nmol/min/mg | (nmol/min/mg | C5/(C5 + C4) | wild type | |
| mutant | cells) | cells) | (%) | DNA | mutant DNA |
| S272N/N86M/S341A | 0.256 | 2.96 | 92.0 | AGC/AAT/AGC | AAC/ATG/GCC |
| S272N/N86M/S341C | 0.368 | 2.991 | 89.0 | | AAC/ATG/TGC |
| S272N/N86M/S341D | 0.285 | 3.018 | 91.4 | | AAC/ATG/GAC |
| S272N/N86M/S341F | 0.248 | 2.006 | 89.0 | | AAC/ATG/TTC |
| S272N/N86M/S341H | 0.220 | 2.762 | 92.6 | | AAC/ATG/CAC |
| S272N/N86M/S341I | 0.121 | 1.188 | 90.8 | | AAC/ATG/ATC |
| S272N/N86M/S341K | 0.037 | 0.445 | 92.3 | | AAC/ATG/AAG |
| S272N/N86M/S341L | 0.145 | 1.042 | 87.8 | | AAC/ATG/CTA |
| S272N/N86M/S341M | 0.207 | 2.161 | 91.3 | | AAC/ATG/ATG |
| S272N/N86M/S341N | 0.397 | 1.359 | 77.4 | | AAC/ATG/AAC |
| S272N/N86M/S341P | 0.060 | 0.162 | 73.0 | | AAC/ATG/CCC |
| S272N/N86M/S341Q | 0.027 | 0.209 | 88.6 | | AAC/ATG/CAG |
| S272N/N86M/S341R | 0.012 | 0.113 | 90.4 | | AAC/ATG/AGG |
| S272N/N86M/S341T | 0.173 | 1.867 | 91.5 | | AAC/ATG/ACC |
| S272N/N86M/S341V | 0.154 | 1.332 | 89.6 | | AAC/ATG/GTC |
| S272N/N86M/S341W | 0.299 | 2.317 | 88.6 | | AAC/ATG/TGG |
| S272N/N86M/S341Y | 0.205 | 1.849 | 90.0 | | AAC/ATG/TAT |
| S272T/N86F/S341A | 0.085 | 1.968 | 95.9 | | ACC/TTT/GCC |
| S272T/N86F/S341C | 0.134 | 2.450 | 94.8 | | ACC/TTT/TGC |
| S272T/N86F/S341D | 0.130 | 2.036 | 94.0 | | ACC/TTT/GAC |
| S272T/N86F/S341E | 0.087 | 1.923 | 95.7 | | ACC/TTT/GAG |
| S272T/N86F/S341F | 0.138 | 1.884 | 93.2 | | ACC/TTT/TTC |
| S272T/N86F/S341H | 0.077 | 2.891 | 97.4 | | ACC/TTT/CAC |
| S272T/N86F/S341K | 0.036 | 0.917 | 96.2 | | ACC/TTT/AAG |
| S272T/N86F/S341L | 0.126 | 1.569 | 92.6 | | ACC/TTT/CTA |
| S272T/N86F/S341M | 0.096 | 2.169 | 95.8 | | ACC/TTT/ATG |
| S272T/N86F/S341N | 0.124 | 1.006 | 89.0 | | ACC/TTT/AAC |
| S272T/N86F/S341P | 0.086 | 0.446 | 83.8 | | ACC/TTT/CCC |
| S272T/N86F/S341Q | 0.133 | 2.852 | 95.5 | | ACC/TTT/CAG |
| S272T/N86F/S341R | 0.039 | 0.549 | 93.4 | | ACC/TTT/AGG |
| S272T/N86F/S341T | 0.095 | 2.008 | 95.5 | | ACC/TTT/ACC |
| S272T/N86F/S341V | 0.091 | 1.769 | 95.1 | | ACC/TTT/GTC |

### Example 4: Production of Gamma-Lactones

A cytochrome P450 monooxygenase gene (GenBank: GAN03094.1) from Mucor ambiguus that confers the activity of hydroxylating fatty acids at the γ-(C4-) position on the E. coli cells overexpressing this gene was used. After hydroxylation, these γ-hydroxy fatty acids can spontaneously form the corresponding gamma-lactones under acidic conditions.

The Genbank GAN03094.1 NADPH-cytochrome P450 reductase [Mucor ambiguus](SEQ ID NO: 1) was codon optimized for Escherichia coli genome and synthesized by Gene Universal Inc. (Newark, DE). The resulting gene SEQ ID NO: 2 was cloned into pET17b vector (AMP⁺, Novagen) through HindIII and XhoI sites. The construct was transformed into BL21(DE3) cells for expression.

In a typical experiment, an overnight culture was used to inoculate liquid LB medium (2%) containing 100 mg/L of carbenicillin and 0.4 mM 5-aminolevulinic acid. The culture was first grown at 37° C. to an OD600 of 0.6 and cooled down to 16° C. Then 1 mM IPTG was added to induce protein expression. After 16 h of incubation at 16° C., cells were harvested by centrifugation.

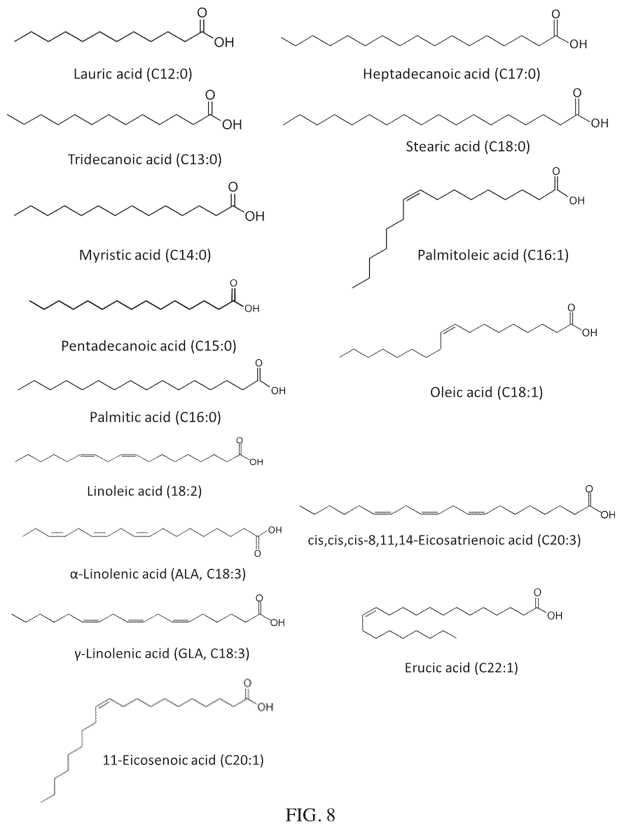

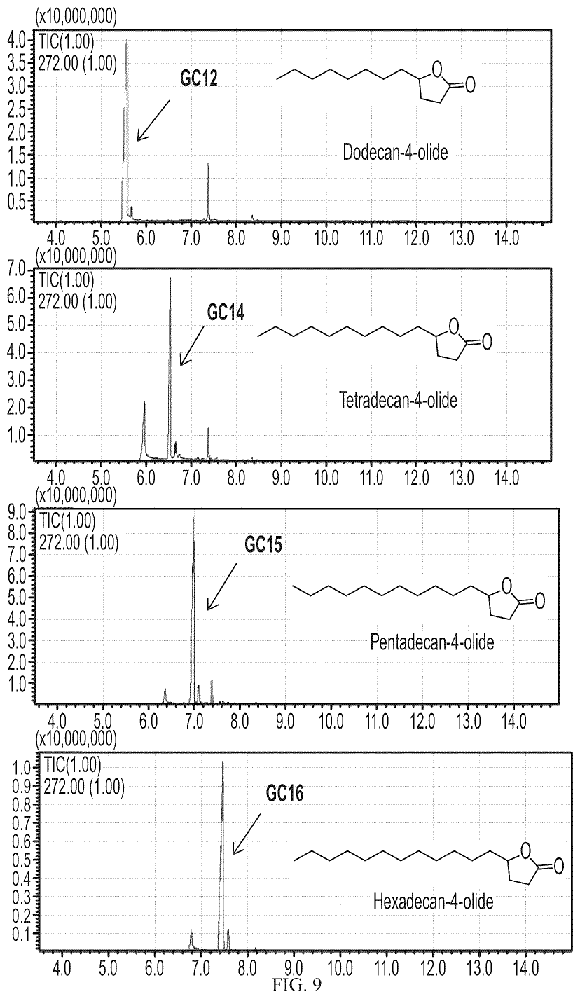

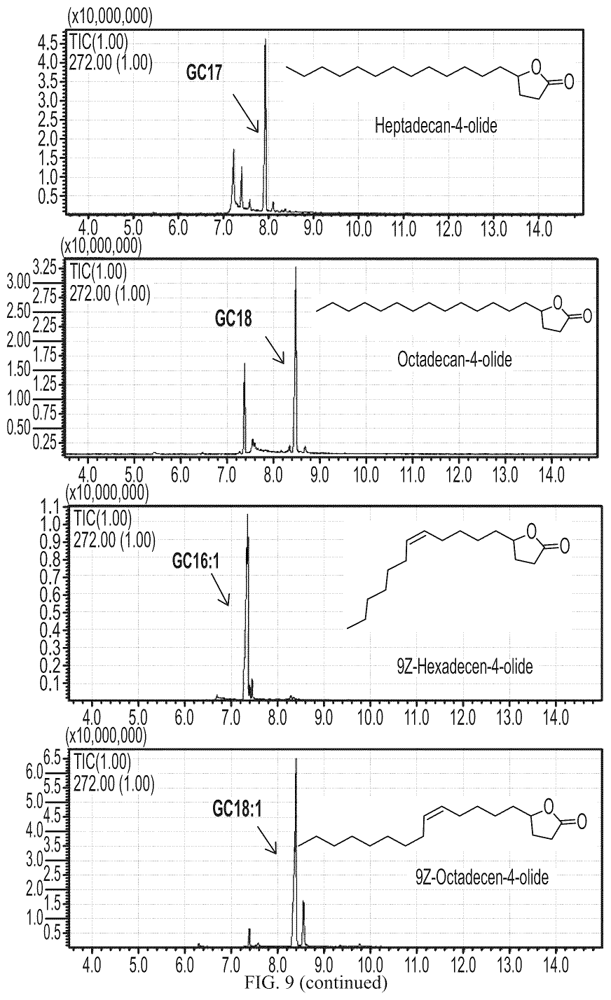

Harvested cell pellets were re-suspended at a concentration of 100 g/L fresh weight in 100 mM potassium phosphate buffer (pH7.0) containing 0.1% Tween 40 and 10 mM NADPH. Then 1 g/L or 2 g/L of various fatty acids (FIG. 8) were added. The mixture was shaken at 37° C. in a shaker (250 rpm).

Samples were taken 5 h after bioconversion and acidified with 2 N HCl to pH 2 for lactone formation. Lactones were extracted by ethyl acetate and ethyl acetate phase was analyzed by GC/MS.

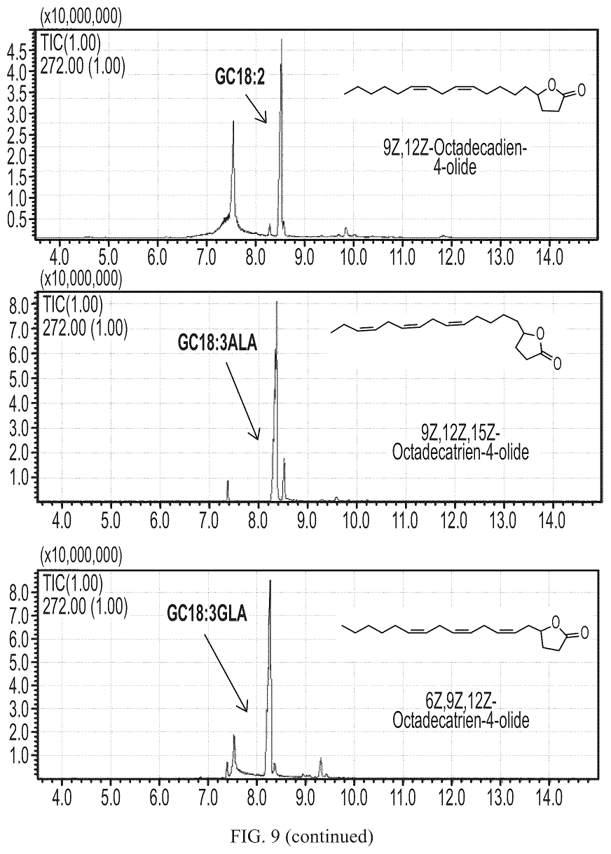

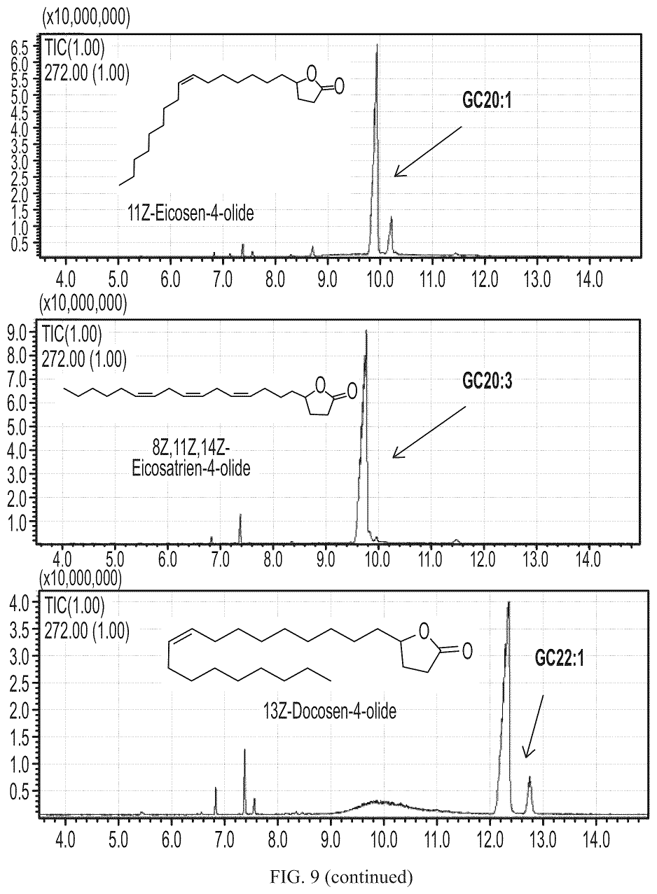

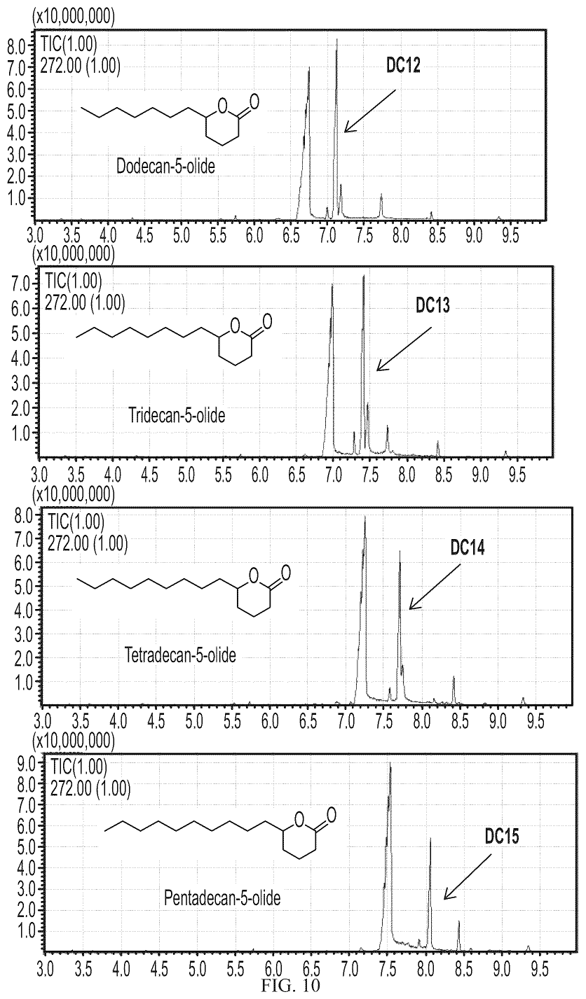

GC/MS analysis was conducted on Shimadzu GC-2030 system coupled with GCMS-QP2020NX detector. The analytical column is SHRXI-5MS (thickness 0.25 μm; length 30 m; diameter 0.25 mm) and the injection temperature is 265° C. under split mode. The temperature gradient is 0-3 min 150° C.; 3-6.7 min 150° C. to 260° C.; 6.7-15.7 min, 260° C. for longer chain fatty acids (Method 1). Exemplary results are shown in FIG. 9.

### Example 5: Production of Delta-Lactones

The mutant of the above mentioned P450 monooxygenase at S272N was used for hydroxylating fatty acids at the δ-(C5-) position on the E. coli cells overexpressing this mutant.

In a typical experiment, an overnight culture was used to inoculate liquid LB medium (2%) containing 100 mg/L of carbenicillin and 0.4 mM 5-aminolevulinic acid. The culture was first grown at 37° C. to an OD600 of 0.6 and cooled down to 16° C. Then 1 mM IPTG was added to induce protein expression. After 16 h of incubation at 16° C., cells were harvested by centrifugation.

Harvested cell pellets were re-suspended at a concentration of 100 g/L fresh weight in 100 mM potassium phosphate buffer (pH7.0) containing 0.1% Tween 40 and 10 mM NADPH. Then 1 g/L or 2 g/L of various fatty acids (FIG. 8) were added. The mixture was shaken at 37° C. in a shaker (250 rpm).

Samples were taken 5 h after bioconversion and acidified with 2 N HCl to pH 2 for lactone formation. Lactones were extracted by ethyl acetate and ethyl acetate phase was analyzed by GC/MS.

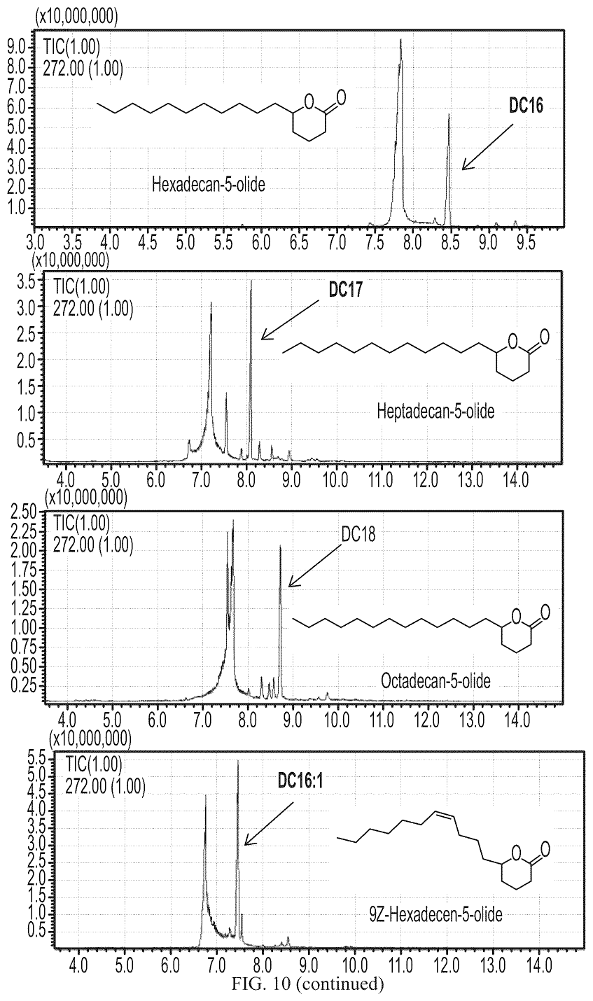

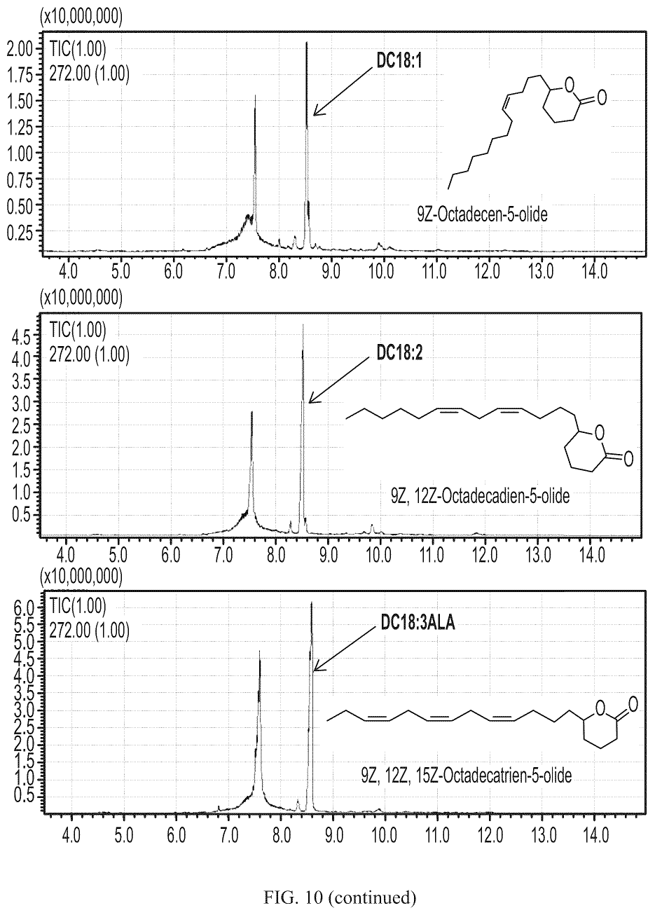

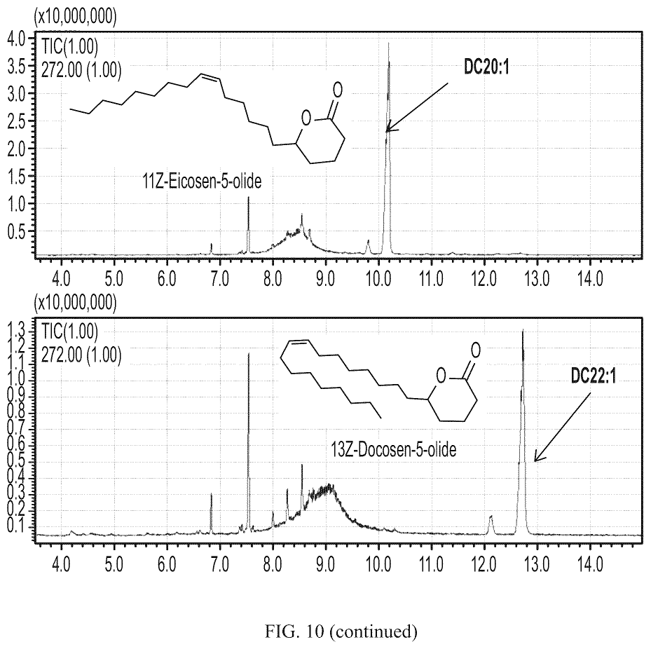

GC/MS analysis was conducted on Shimadzu GC-2030 system coupled with GCMS-QP2020NX detector. The analytical column is SHRXI-5MS (thickness 0.25 μm; length 30 m; diameter 0.25 mm) and the injection temperature is 265° C. under split mode. The temperature gradient is 0-3 min 150° C.; 3-6.7 min 150° C. to 260° C.; 6.7-15.7 min, 260° C. for longer chain fatty acids (Method 1). Or the temperature gradient is 0-3 min 80° C.; 3-8.7 min 80° C. to 263° C.; 8.7-10.7 min, 263° C. for shorter chain fatty acids (Method 2). Exemplary results are shown in FIG. 10.

Although the present disclosure has been described in some detail by way of illustration and example for purposes of understanding, it will be apparent to those skilled in the art that certain changes and modifications may be practiced. Therefore, the description and examples should not be construed as limiting the scope of the present disclosure, which is delineated by the appended claims.

SEQUENCES

SEQ ID NO: 1
Amino acid sequence of the wild-type MaP450 of Mucor Ambiguus (GAN03094.1.):
MTKYAHDQIPGPEPHYLLGNVPDIFPDSLGNLIKLHDKYGPIVHLSMGGHELLSVDDPAV
LETICEDNEYFTKEIESVYSDLAILNGRGLVTTSTADPDWQLGHKLIMNAFSARAMKAY
HYKMGESISELCEIMDSFAKSGEDFDVSRWFIALALESIGKIGFDYDFDLLKDPNAPRHPF
TVALAYVQSMIMKRASTLSWLKWYQTTTNVRFHRDLQTLRGTVEEVLKDRREHPHTEA
DQSDLLDFMIKAESKEGEKLNDSLIRDNIITFLSAGHNTTSAFLSWTMLELCKHPEVVENI
KQEIANCGIKAGEVPTPEQVKECKYLDLVIKESLRIHPPITSILKYCKKDATVKASNGDEY
DIKAGQLLQVNINALHHNPKVWEDPDVFNPDRFSGDTDNLPNTAWLTFSTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDESLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRESLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGESPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 1)
SEQ ID NO: 2
DNA Sequence encoding GAN03094.1 (codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGAATGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGAGCGCAGGTCATAATACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCA
GCATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 2)
SEQ ID NO: 3
Amino acid sequence of the MaP450 of Mucor Ambiguus (GAN03094.1) S272N mutant:
MTKYAHDQIPGPEPHYLLGNVPDIFPDSLGNLIKLHDKYGPIVHLSMGGHELLSVDDPAV
LETICEDNEYFTKEIESVYSDLAILNGRGLVTTSTADPDWQLGHKLIMNAFSARAMKAY
HYKMGESISELCEIMDSFAKSGEDFDVSRWFIALALESIGKIGFDYDFDLLKDPNAPRHPF
TVALAYVQSMIMKRASTLSWLKWYQTTTNVRFHRDLQTLRGTVEEVLKDRREHPHTEA
DQSDLLDFMIKAESKEGEKLNDSLIRDNIITFLNAGHNTTSAFLSWTMLELCKHPEVVENI
KQEIANCGIKAGEVPTPEQVKECKYLDLVIKESLRIHPPITSILKYCKKDATVKASNGDEY
DIKAGQLLQVNINALHHNPKVWEDPDVFNPDRFSGDTDNLPNTAWLTESTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDESLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRESLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGFSPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 3)
SEQ ID NO: 4
Nucleotide sequence encoding MaP450 of Mucor Ambiguus (GAN03094.1) S272N mutant
(codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGAATGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGAACGCAGGTCATAATACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCA
GCATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 4)
SEQ ID NO: 5
Amino acid sequence of the MaP450 of Mucor Ambiguus (GAN03094.1) S272T mutant:
MTKYAHDQIPGPEPHYLLGNVPDIFPDSLGNLIKLHDKYGPIVHLSMGGHELLSVDDPAV
LETICEDNEYFTKEIESVYSDLAILNGRGLVTTSTADPDWQLGHKLIMNAFSARAMKAY
HYKMGESISELCEIMDSFAKSGEDFDVSRWFIALALESIGKIGFDYDFDLLKDPNAPRHPF
TVALAYVQSMIMKRASTLSWLKWYQTTTNVRFHRDLQTLRGTVEEVLKDRREHPHTEA
DQSDLLDFMIKAESKEGEKLNDSLIRDNIITFLTAGHNTTSAFLSWTMLELCKHPEVVENI
KQEIANCGIKAGEVPTPEQVKECKYLDLVIKESLRIHPPITSILKYCKKDATVKASNGDEY
DIKAGQLLQVNINALHHNPKVWEDPDVFNPDRFSGDTDNLPNTAWLTESTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDESLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRESLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGFSPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 5)
SEQ ID NO: 6
Nucleotide sequence encoding MaP450 of Mucor Ambiguus (GAN03094.1) S272T mutant
(codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGAATGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGACCGCAGGTCATAATACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCA
GCATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 6)
SEQ ID NO: 7
Amino acid sequence of the MaP450 of Mucor Ambiguus (GAN03094.1) N276T mutant:
MTKYAHDQIPGPEPHYLLGNVPDIFPDSLGNLIKLHDKYGPIVHLSMGGHELLSVDDPAV
LETICEDNEYFTKEIESVYSDLAILNGRGLVTTSTADPDWQLGHKLIMNAFSARAMKAY
HYKMGESISELCEIMDSFAKSGEDFDVSRWFIALALESIGKIGFDYDFDLLKDPNAPRHPF
TVALAYVQSMIMKRASTLSWLKWYQTTTNVRFHRDLQTLRGTVEEVLKDRREHPHTEA
DQSDLLDFMIKAESKEGEKLNDSLIRDNIITFLSAGHTTTSAFLSWTMLELCKHPEVVENI
KQEIANCGIKAGEVPTPEQVKECKYLDLVIKESLRIHPPITSILKYCKKDATVKASNGDEY
DIKAGQLLQVNINALHHNPKVWEDPDVFNPDRFSGDTDNLPNTAWLTFSTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDESLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRESLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGFSPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 7)
SEQ ID NO: 8
Nucleotide sequence encoding MaP450 of Mucor Ambiguus (GAN03094.1) N276T mutant
(codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGAATGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGAGCGCAGGTCATACTACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCA
GCATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 8)
SEQ ID NO: 9
Amino acid sequence of the MaP450 of Mucor Ambiguus (GAN03094.1) S272N/N86M/S341D
mutant:
MTKYAHDQIPGPEPHYLLGNVPDIFPDSLGNLIKLHDKYGPIVHLSMGGHELLSVDDPAV
LETICEDNEYFTKEIESVYSDLAILMGRGLVTTSTADPDWQLGHKLIMNAFSARAMKAY
HYKMGESISELCEIMDSFAKSGEDFDVSRWFIALALESIGKIGFDYDFDLLKDPNAPRHPF
TVALAYVQSMIMKRASTLSWLKWYQTTTNVRFHRDLQTLRGTVEEVLKDRREHPHTEA
DQSDLLDFMIKAESKEGEKLNDSLIRDNIITFLNAGHNTTSAFLSWTMLELCKHPEVVENI
KQEIANCGIKAGEVPTPEQVKECKYLDLVIKESLRIHPPITDILKYCKKDATVKASNGDEY
DIKAGQLLQVNINALHHNPKVWEDPDVFNPDRESGDTDNLPNTAWLTFSTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDFSLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRESLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGFSPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 9)
SEQ ID NO: 10
Nucleotide sequence encoding MaP450 of Mucor Ambiguus (GAN03094.1)
S272N/N86M/S341D mutant (codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGATGGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGAACGCAGGTCATAATACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCG
ACATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 10)
SEQ ID NO: 11
Amino acid sequence of the MaP450 of Mucor Ambiguus (GAN03094.1) S272T/N86F/S341H
mutant:
MTKYAHDQIPGPEPHYLLGNVPDIFPDSLGNLIKLHDKYGPIVHLSMGGHELLSVDDPAV
LETICEDNEYFTKEIESVYSDLAILFGRGLVTTSTADPDWQLGHKLIMNAFSARAMKAYH
YKMGESISELCEIMDSFAKSGEDFDVSRWFIALALESIGKIGFDYDFDLLKDPNAPRHPFT
VALAYVQSMIMKRASTLSWLKWYQTTTNVRFHRDLQTLRGTVEEVLKDRREHPHTEA
DQSDLLDFMIKAESKEGEKLNDSLIRDNIITFLTAGHNTTSAFLSWTMLELCKHPEVVENI
KQEIANCGIKAGEVPTPEQVKECKYLDLVIKESLRIHPPITHILKYCKKDATVKASNGDEY
DIKAGQLLQVNINALHHNPKVWEDPDVFNPDRFSGDTDNLPNTAWLTESTGPRACIGRQ
FALQEGKLALVMILSRFHFKMDDPSQKIGYAVVVSTKPVGFFAKIESSQLPEPTEEIVVTK
RRESKAVPQEKVKPAEFPLPPVTFLFGTQTNTSEEYARKLSGQAKEMGFKEVTVQDLDD
WKLVKGEAIAKAQHDADAPSSEDDVKVSELVVVVTATYNGFPPDDANEFAKWLDERT
KDSEATKNNMLSGMLYAVFGCGNRDWTSTFQKFPKKVDSGFELLGGERLLPAGEGDAS
DDIDGDESLWSASFWTALMQRYGQSSSGKNADIMSNNGPAADPSQDFTLEFINIVKEKV
KTEQAALNCNQLETVATIVENRELQHTEKSHRSTRHIQVQFDKSVDGKPLYEAGDHLEV
VPVNEDRLVEIIATNLGLVLDSVFEVKDLDIKNLSPRSVAANIKGPCTIRNALKYYADLT
GPPTRFSLSILSKQLKDSRPDIAERLQKALQPGKETERLKEFLASHRTLIDIIQAFKIKELNF
KEFISSVNCIVPRKYSISSGPLEHPFDPSISVGVVDTVGGPDGNTHYFGLASGYLSHQEPGT
KINAQIKACKSTFRLPDDPSTPVIFIAAGTGFSPFRGFLQERHAKGLKSSKKNSNGESSECY
MFFGCRHPDQDFIYKEEFDAYLEDGTITELYTTFSRSGEVVKYVQHALLKHANLLYKLM
EESNAKVYICGSAGSMAKDVKRTWERLLVQMSGVSESEAAEQIQAWVDEGKYNEDVW
GT (SEQ ID NO: 11)
SEQ ID NO: 12
Nucleotide sequence encoding MaP450 of Mucor Ambiguus (GAN03094.1) S272T/
N86F/S341H mutant (codon optimized for Escherichia coli)
ATGACCAAATATGCCCATGATCAGATTCCGGGTCCTGAACCGCATTATCTGCTGGGT
AATGTTCCGGATATTTTTCCGGATAGCCTGGGCAATCTGATTAAACTGCATGATAAA
TATGGTCCGATTGTGCATCTGAGCATGGGTGGTCATGAACTGCTGAGCGTTGATGAT
CCGGCAGTTCTGGAAACCATTTGTGAAGATAATGAATATTTCACCAAAGAAATCGAA
AGCGTGTATAGCGATCTGGCAATTCTGTTTGGTCGTGGTCTGGTTACCACCAGTACC
GCAGATCCGGATTGGCAGCTGGGTCATAAACTGATTATGAATGCATTTAGCGCACGT
GCCATGAAAGCCTATCACTATAAAATGGGTGAAAGCATTAGCGAACTGTGCGAAAT
TATGGATAGCTTTGCAAAAAGCGGTGAGGATTTTGATGTTAGCCGTTGGTTTATTGC
ACTGGCACTGGAAAGCATTGGTAAAATCGGTTTCGATTATGATTTCGACCTGCTGAA
AGATCCGAATGCACCGCGTCATCCGTTTACCGTTGCGCTGGCCTATGTTCAGAGTAT
GATCATGAAACGTGCAAGCACCCTGAGCTGGCTGAAATGGTATCAGACCACCACCA
ATGTTCGTTTTCATCGTGATCTGCAGACCCTGCGTGGCACCGTTGAAGAAGTTCTGA
AAGACCGTCGTGAACATCCGCATACCGAAGCAGATCAGAGCGATCTGCTGGATTTTA
TGATTAAAGCCGAAAGCAAAGAAGGCGAGAAACTGAACGATAGCCTGATTCGTGAT
AACATCATTACCTTTCTGACCGCAGGTCATAATACCACCTCAGCATTTCTGAGCTGG
ACCATGCTGGAACTGTGTAAACATCCGGAAGTTGTCGAAAACATCAAACAAGAAAT
TGCCAACTGCGGTATTAAAGCGGGTGAAGTTCCGACACCGGAACAGGTTAAAGAAT
GTAAATATCTGGACCTGGTGATCAAAGAAAGCCTGCGTATTCATCCGCCTATTACCC
ACATTCTGAAATACTGTAAAAAAGACGCAACCGTGAAAGCCAGCAATGGTGATGAA
TATGATATTAAAGCAGGTCAGCTGCTGCAGGTTAACATTAATGCACTGCATCATAAC
CCGAAAGTTTGGGAAGATCCTGATGTTTTTAACCCGGATCGTTTTAGCGGTGATACC
GATAATCTGCCGAATACCGCATGGCTGACCTTTAGCACCGGTCCGCGTGCATGTATT
GGTCGTCAGTTTGCACTGCAAGAAGGTAAACTGGCCCTGGTTATGATTCTGAGCCGT
TTTCATTTCAAAATGGATGATCCGAGCCAGAAAATTGGTTATGCAGTTGTTGTTAGC
ACCAAACCGGTTGGTTTTTTTGCCAAAATTGAAAGCAGCCAGCTGCCGGAACCGACC
GAAGAAATTGTTGTTACCAAACGTCGTGAAAGTAAAGCAGTTCCGCAAGAAAAAGT
TAAACCGGCAGAATTTCCGCTGCCTCCGGTGACCTTTCTGTTTGGCACCCAGACCAA
TACCAGCGAAGAATATGCACGTAAACTGAGCGGTCAGGCAAAAGAAATGGGTTTTA
AAGAAGTTACCGTCCAGGATCTGGATGATTGGAAACTGGTTAAAGGTGAAGCAATT
GCAAAAGCACAGCATGATGCCGATGCACCGAGCAGCGAAGATGATGTTAAAGTGAG
CGAACTGGTTGTTGTTGTGACCGCAACCTATAATGGTTTTCCGCCTGATGATGCAAA
CGAATTTGCAAAATGGCTGGATGAACGTACCAAAGATAGCGAAGCAACCAAAAATA
ACATGCTGAGCGGTATGCTGTATGCAGTGTTTGGTTGTGGTAATCGTGATTGGACCA
GCACCTTTCAGAAATTCCCGAAAAAAGTTGATAGCGGCTTTGAACTGTTAGGTGGTG
AACGTCTGCTGCCAGCCGGTGAAGGTGATGCAAGTGATGATATTGATGGTGATTTTA
GCCTGTGGTCTGCCAGCTTTTGGACCGCACTGATGCAGCGTTATGGTCAGAGCAGCA
GCGGTAAAAATGCAGATATTATGAGCAATAATGGTCCGGCAGCAGATCCGAGTCAG
GATTTTACCCTGGAATTTATCAACATCGTGAAAGAGAAGGTCAAAACCGAACAGGC
AGCACTGAATTGTAATCAGCTGGAAACCGTTGCAACCATTGTTGAAAATCGCGAACT
GCAGCATACAGAAAAAAGCCATCGTAGCACCCGTCATATTCAGGTTCAGTTTGATAA
AAGCGTGGATGGTAAACCGCTGTATGAAGCCGGTGATCATCTGGAAGTGGTTCCGGT
TAATGAAGATCGTCTGGTTGAAATTATTGCCACCAATCTGGGTTTAGTTCTGGATAG
CGTTTTTGAGGTGAAAGACCTGGATATTAAGAATCTGAGTCCGCGTAGCGTTGCAGC
AAACATTAAAGGTCCGTGTACCATTCGTAATGCCCTGAAATATTACGCAGATCTGAC
CGGTCCTCCGACACGTTTTAGTCTGAGCATTCTGTCAAAACAGCTGAAAGACAGCCG
TCCTGATATTGCAGAACGCCTGCAGAAAGCACTGCAGCCTGGTAAAGAAACCGAAC
GTCTGAAAGAATTTCTGGCAAGTCATCGTACCCTGATCGATATTATTCAGGCCTTCA
AAATCAAAGAACTGAACTTCAAAGAGTTTATCAGCAGCGTTAATTGCATCGTTCCGC
GTAAATATAGCATTAGCAGTGGTCCGCTGGAACATCCGTTTGATCCGAGTATTAGCG
TTGGTGTTGTTGATACCGTTGGTGGTCCGGATGGTAATACCCATTATTTTGGTCTGGC
AAGCGGTTATCTGAGCCATCAAGAACCGGGTACAAAAATCAATGCACAGATTAAAG
CATGCAAGAGTACCTTTCGTCTGCCGGATGATCCTAGCACACCGGTTATCTTTATTGC
AGCAGGCACCGGTTTTAGCCCGTTTCGTGGTTTTCTGCAAGAACGTCATGCAAAAGG
TCTGAAAAGCAGCAAAAAAAACAGCAATGGCGAAAGCAGCGAGTGCTATATGTTTT
TTGGCTGTCGTCATCCGGATCAGGATTTCATCTATAAAGAAGAATTTGACGCCTACC
TGGAAGATGGCACCATTACAGAACTGTATACCACCTTTAGCCGTAGCGGTGAAGTTG
TTAAATATGTTCAGCACGCACTGCTGAAACATGCAAATCTGCTGTACAAACTGATGG
AAGAAAGCAACGCCAAAGTGTATATTTGTGGTAGCGCAGGTAGCATGGCAAAAGAT
GTTAAACGTACCTGGGAGCGCCTGCTGGTTCAGATGAGCGGTGTTAGCGAAAGCGA
AGCAGCAGAGCAGATTCAGGCATGGGTTGATGAAGGCAAATATAACGAAGATGTTT
GGGGCACCTAA (SEQ ID NO: 12)

### ADDITIONAL EMBODIMENTS

1. A bioconversion method for the production of a delta-lactone, the bioconversion method comprising:
growing a cellular system in a culture medium, wherein the cellular system comprises a host cell which has been modified to express a recombinant cytochrome P450 hydroxylase polypeptide comprising one or more amino acid substitutions at positions N86, S272 and S341 in SEQ ID NO: 1;
expressing the recombinant cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising five to thirty-four carbon atoms, a salt thereof, an alkyl ester thereof, a mono, di or triglyceride thereof or an unsubstituted monoalkyl or dialkyl amide thereof, thereby producing the delta-lactone in a recoverable amount.
   2. The bioconversion method of embodiment 1, wherein said substrate is a carboxylic acid comprising a linear or branched, alkyl or alkenyl moiety comprising five to fifteen carbon atoms, a salt thereof, an alkyl ester thereof, a mono, di or triglyceride thereof or an unsubstituted monoalkyl or dialkyl amide thereof.
   3. A bioconversion method for the production of a gamma-lactone, the bioconversion method comprising:
growing a cellular system in a culture medium, wherein the cellular system comprises a host cell which has been modified to express a cytochrome P450 hydroxylase polypeptide comprising an amino acid sequence at least 70% identical to that of SEQ ID NO: 1;
expressing the cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising five to thirty-four carbon atoms, a salt thereof, an alkyl ester thereof, a mono, di or triglyceride thereof or an unsubstituted monoalkyl or dialkyl amide thereof, thereby producing the gamma-lactone in a recoverable amount.
   4. The bioconversion method of any one of embodiments 1-3, wherein the alkyl or alkenyl moiety of the substrate comprises an unbranched chain comprising at least five carbon atoms, wherein one of the at least five carbon atoms is linked to a carboxylic acid moiety.
   5. The bioconversion method of any one of embodiments 1-2 and 4, wherein the hydroxylase polypeptide converts a carboxylic acid substrate into a 5-hydroxy fatty acid.
   6. The bioconversion method of any one of embodiments 3-4, wherein the hydroxylase polypeptide converts a carboxylic acid substrate into a 4-hydroxy fatty acid.
   7. The bioconversion method of any one of embodiments 1-6, wherein said host cell is a bacterium, a yeast cell, a fungal cell, an alga cell, or a plant cell.
   8. The bioconversion method of any one of embodiments 1-6, wherein the host cell is bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium.
   9. The bioconversion method of any one of embodiments 1-6, wherein the host cell is a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys.
   10. The bioconversion method of any one of embodiments 1-6, wherein the host cell is E. Coli.
   11. The bioconversion method of any one of embodiments 1-10, wherein the substrate is lauric acid.
   12. The bioconversion method of any one of embodiments 1-11 further comprising acidifying the culture medium.
   13. A cytochrome P450 polypeptide comprises one or more amino acid substitutions at positions N86, S272 and S341 in SEQ ID NO: 1.
   14. The bioconversion method of any one of embodiments 1-2, 4-5, and 7-12 or the cytochrome P450 polypeptide of embodiment 13, wherein the cytochrome P450 hydroxylase polypeptide comprises an amino acid substitution selected from S272I, S272L, S272M, S272N, S272T and N276T.
   15. The bioconversion method of any one of embodiments 1-2, 4-5, and 7-12 or the cytochrome P450 polypeptide of embodiment 13, wherein the cytochrome P450 hydroxylase polypeptide comprises amino acid substitutions selected from S272N/N86E, S272N/N86M, S272N/S341G, S272N/S341H, S272N/S341N, S272T/N86F, S272T/N86I, S272T/N86V.
   16. The bioconversion method of any one of embodiments 1-2, 4-5, and 7-12 or the cytochrome P450 polypeptide of embodiment 13, wherein the cytochrome P450 hydroxylase polypeptide comprises amino acid substitutions selected from S272N/N86M/S341D, S272N/N86M/S341H, S272T/N86F/S341A, S272T/N86F/S341C, S272T/N86F/S341H, S272T/N86F/S341M, S272T/N86F/S341Q.
   17. The bioconversion method of any one of embodiments 1-2, 4-5, and 7-16 or the cytochrome P450 polypeptide of embodiment 13, wherein the cytochrome P450 hydroxylase polypeptide comprises an amino acid sequence at least 90% identical to that of SEQ ID NOs: 3, 5, 7, 9, or 11.
   18. The bioconversion method of any one of embodiments 1-2, 4-5, and 7-16 or the cytochrome P450 polypeptide of embodiment 13, wherein the cytochrome P450 hydroxylase polypeptide comprises the amino acid sequence of any one of SEQ ID NOs: 3, 5, 7, 9, or 11.
   19. The cytochrome P450 polypeptide of any one of embodiments 13-18, wherein the cytochrome P450 polypeptide of is capable of converting a fatty acid to a delta lactone.
   20. The cytochrome P450 polypeptide of embodiment 19, wherein the delta-lactone has a purity of not less than 50%.
   21. The cytochrome P450 polypeptide of embodiment 19, wherein the delta-lactone product has a purity of not less than 75%.
   22. A nucleic acid molecule comprising a nucleotide sequence encoding the cytochrome P450 polypeptide of any one of embodiments 13-21.
   23. The nucleic acid molecule of embodiment 22, wherein the nucleotide sequence is any one of SEQ ID NOs. 4, 6, 8, 10, or 12.
   24. A host cell comprising a vector capable of producing the cytochrome P450 polypeptide of any one of embodiments 13-21.
   25. A bioenzymatic method for the production of a delta-lactone, the bioenzymatic method comprising:
incubating a cytochrome P450 polypeptide comprising one or more amino acid substitutions at positions N86, S272 and S341 in SEQ ID NO: 1, with a fatty acid substrate and NADPH for a sufficient time to convert the fatty acid substrate to a hydroxylated fatty acid composition comprising one or more hydroxylated fatty acids, wherein a delta-hydroxylated fatty acid is present at a ratio of at least 20% of all hydroxylated fatty acids present in the hydroxylated fatty acid composition; and
acidifying the hydroxylated fatty acid composition to convert the delta-hydroxylated fatty acid to a delta-lactone.
   26. A bioenzymatic method for the production of a gamma-lactone, the bioenzymatic method comprising:
incubating a cytochrome P450 hydroxylase polypeptide comprising an amino acid sequence at least 70% identical to that of SEQ ID NO: 1, with a fatty acid substrate and NADPH for a sufficient time to convert the fatty acid substrate to a hydroxylated fatty acid composition comprising one or more hydroxylated fatty acids, wherein a gamma-hydroxylated fatty acid is present at a ratio of at least 20% of all hydroxylated fatty acids present in the hydroxylated fatty acid composition; and
acidifying the hydroxylated fatty acid composition to convert the gamma-hydroxylated fatty acid to a gamma-lactone.
   27. The bioconversion method of any one of embodiments 1-12 and 14-18 or the bioenzymatic method of any one of embodiments 25-26, wherein the cytochrome P450 hydroxylase polypeptide comprises an amino acid sequence at least 90% identical to that of SEQ ID NO: 1.
   28. The bioconversion method of any one of embodiments 1-12, 14-18, and 27 or the bioenzymatic method of any one of embodiments 25-27, wherein the fatty acid substrate is (unsubstituted, branched or unbranched, C₈₋₃₄ alkyl)-C(═O)OH, (unsubstituted, branched or unbranched, C₈₋₃₄ alkenyl)-C(═O)OH, or (unsubstituted, branched or unbranched, C₈₋₃₄ alkynyl)-C(═O)OH, or a tautomer, isotopically labeled compound, salt, solvate, polymorph, or co-crystal thereof.
   29. The bioconversion method of any one of embodiments 1-12, 14-18, and 27 or the bioenzymatic method of embodiment 25-27, wherein the fatty acid substrate is represented by Formula (I):

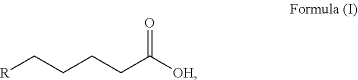

wherein R is a C₄₋₁₂ alkyl group, a C₄₋₁₂ alkenyl, or a C₄₋₁₂ alkynyl group.
   30. The bioconversion method of any one of embodiments 1-2, 4-5, 7-12, 14-18, and 27 or the bioenzymatic method of embodiment 25 and 27, wherein the delta-hydroxylated fatty acid is represented by Formula (II):

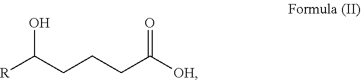

and the delta-lactone is represented by Formula (III):

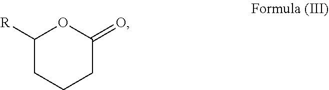

wherein R is a C₄₋₁₂ alkyl group, a C₄₋₁₂ alkenyl group, or a C₄₋₁₂ alkynyl group.
   31. The bioconversion method of any one of embodiments 1-2, 4-5, 7-12, 14-18, and 27 or the bioenzymatic method of any one of embodiments 25 and 27, wherein the product delta-lactone is delta-dodecalactone.
   32. The bioconversion method of any one of embodiments 1-2, 4-5, 7-12, 14-18, and 27 or the bioenzymatic method of any one of embodiments 25 and 27, wherein the product delta-lactone is selected from the group consisting of delta-heptalactone, delta-octalactone, delta-nonalactone, delta-decalactone, delta-undecalactone, delta-dodecalactone, delta-tridecalactone, delta-tetradecalactone, and mixtures thereof.
   33. The bioconversion method of any one of embodiments 1-2, 4-5, 7-12, 14-18, and 27 or the bioenzymatic method of any one of embodiments 25 and 27, wherein the product delta-lactone is represented by Formula (V):

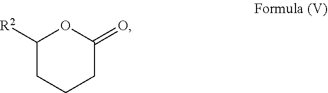

or a tautomer, isotopically labeled compound, solvate, polymorph, or co-crystal thereof, or a mixture thereof, wherein R² is unsubstituted, branched or unbranched, C₄₋₃₀ alkyl, C₄ 30 alkenyl, or C₄₋₃₀ alkynyl.
   34. The bioconversion method of any one of embodiments 3-4, 6-12, and 27 or the bioenzymatic method of any one of embodiments 26-27, wherein the product gamma-lactone is represented by Formula (VI):

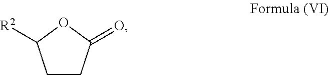

or a tautomer, isotopically labeled compound, solvate, polymorph, or co-crystal thereof, or a mixture thereof, wherein R² is unsubstituted, branched or unbranched, C₄₋₃₀ alkyl, C₄ 30 alkenyl, or C₄₋₃₀ alkynyl.
   35. The bioconversion method of any one of embodiments 33-34 or bioenzymatic method of any one of embodiments 33-34, wherein R² is unsubstituted, branched or unbranched, C₄-30 alkyl.
   36. The bioconversion method of any one of embodiments 33-34 or bioenzymatic method of embodiment 33-34, wherein R² is unsubstituted unbranched C₄₋₂₄ alkyl.
   37. The bioconversion method of any one of embodiments 33-34 or bioenzymatic method of any one of embodiments 33-34, wherein R² is unsubstituted, branched or unbranched, C₄₋₃₀ alkenyl.
   38. The bioconversion method of any one of embodiments 33-34 or bioenzymatic method of embodiment 33-34, wherein R² is unsubstituted unbranched C₆₋₂₄ alkenyl.
   39. The bioconversion method of any one of embodiments 1-12, 14-18, and 27-38 or bioenzymatic method of any one of embodiments 25-38, wherein the product delta-lactone and/or product gamma-lactone do not comprise C═C═C.
   40. The bioconversion method of any one of embodiments 1-12, 14-18, and 27-39 or bioenzymatic method of any one of embodiments 25-39, wherein the product delta-lactone and/or product gamma-lactone do not comprise C≡C.
   41. The bioconversion method of any one of embodiments 1-12, 14-18, and 27-40 or bioenzymatic method of any one of embodiments 25-40, wherein each double bond of the alkenyl is a Z double bond.
   42. The bioconversion method of any one of embodiments 1-12, 14-18, 27-34, and 37-41 or bioenzymatic method of any one of embodiments 25-41, wherein R² comprises only one double bond.
   43. The bioconversion method of any one of embodiments 1-12, 14-18, 27-34, and 37-41 or bioenzymatic method of any one of embodiments 25-41, wherein R² comprises only two double bonds.
   44. The bioconversion method of any one of embodiments 1-12, 14-18, 27-34, and 37-41 or bioenzymatic method of any one of embodiments 25-41, wherein R² comprises only three double bonds.
   45. The bioconversion method of any one of embodiments 1-12, 14-18, 27-34, and 37-41 or bioenzymatic method of any one of embodiments 25-41, wherein R² comprises only four double bonds.
   46. The bioconversion method of embodiment 33 or the bioenzymatic method embodiment 33, wherein the product delta-lactone is of the formula:

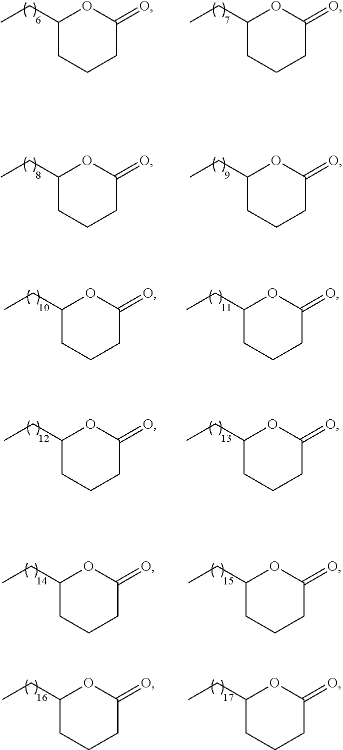

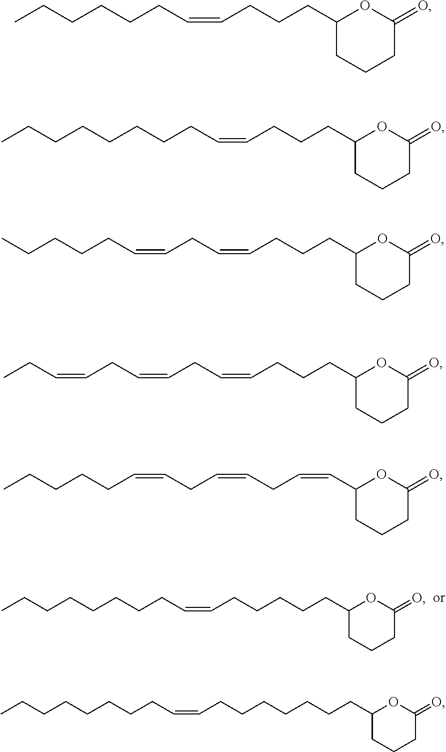

or a tautomer, isotopically labeled compound, solvate, polymorph, or co-crystal thereof; or a mixture thereof.
   47. The bioconversion method of embodiment 34 or the bioenzymatic method embodiment 34, wherein the product gamma-lactone is of the formula:

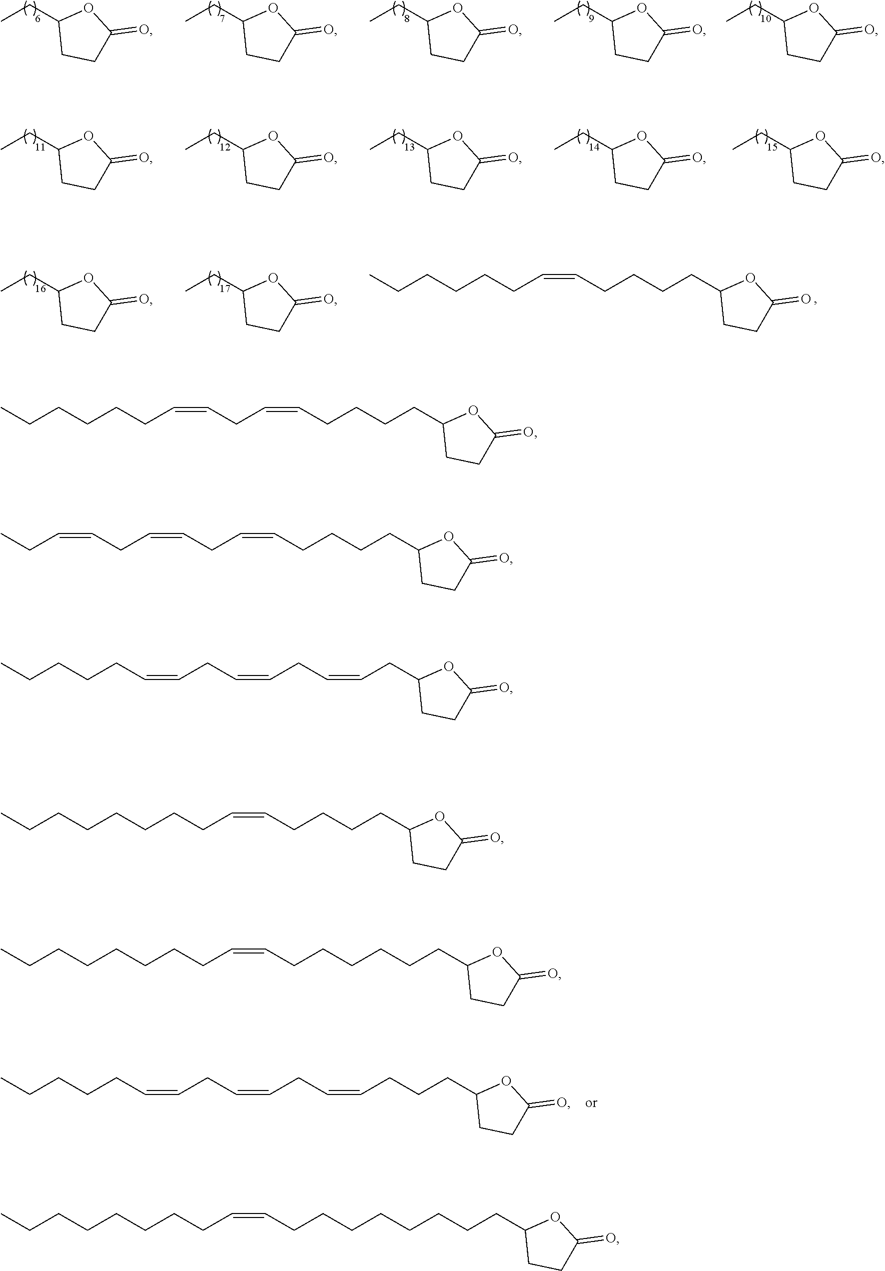

or a tautomer, isotopically labeled compound, solvate, polymorph, or co-crystal thereof; or a mixture thereof.
   48. A gamma- or delta-lactone represented by Formula (IV) or (1):

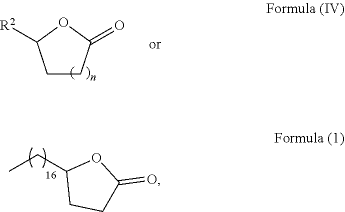

or a tautomer, isotopically labeled compound, solvate, polymorph, or co-crystal thereof, wherein:
   n is 1 or 2; and
   R¹ is unsubstituted unbranched C₇₋₁₈ alkenyl, wherein each double bond of the unsubstituted unbranched C₇₋₁₈ alkenyl is a Z double bond;
provided that the gamma- or delta-lactone does not comprise C═C═C or C≡C and is not represented by the formula:

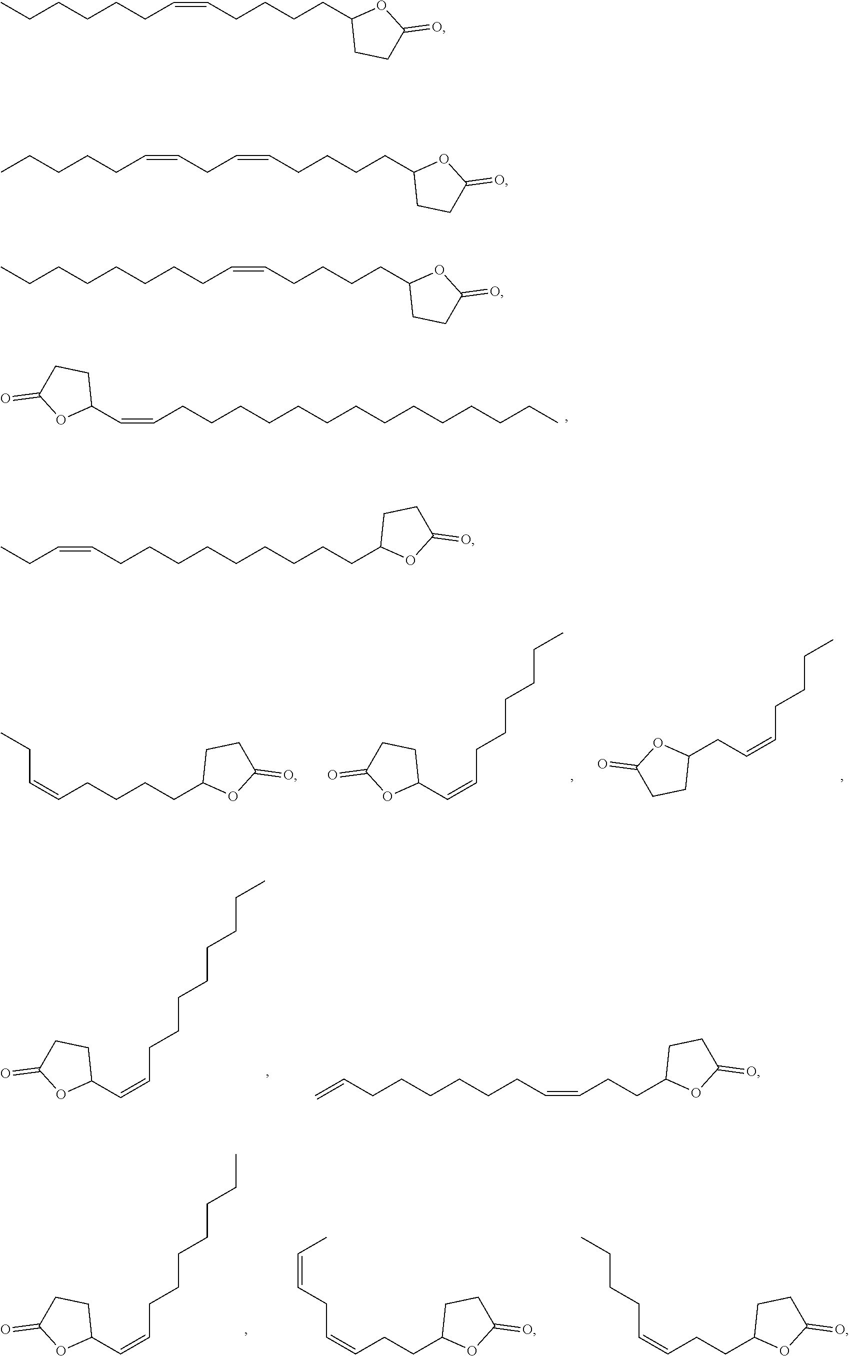

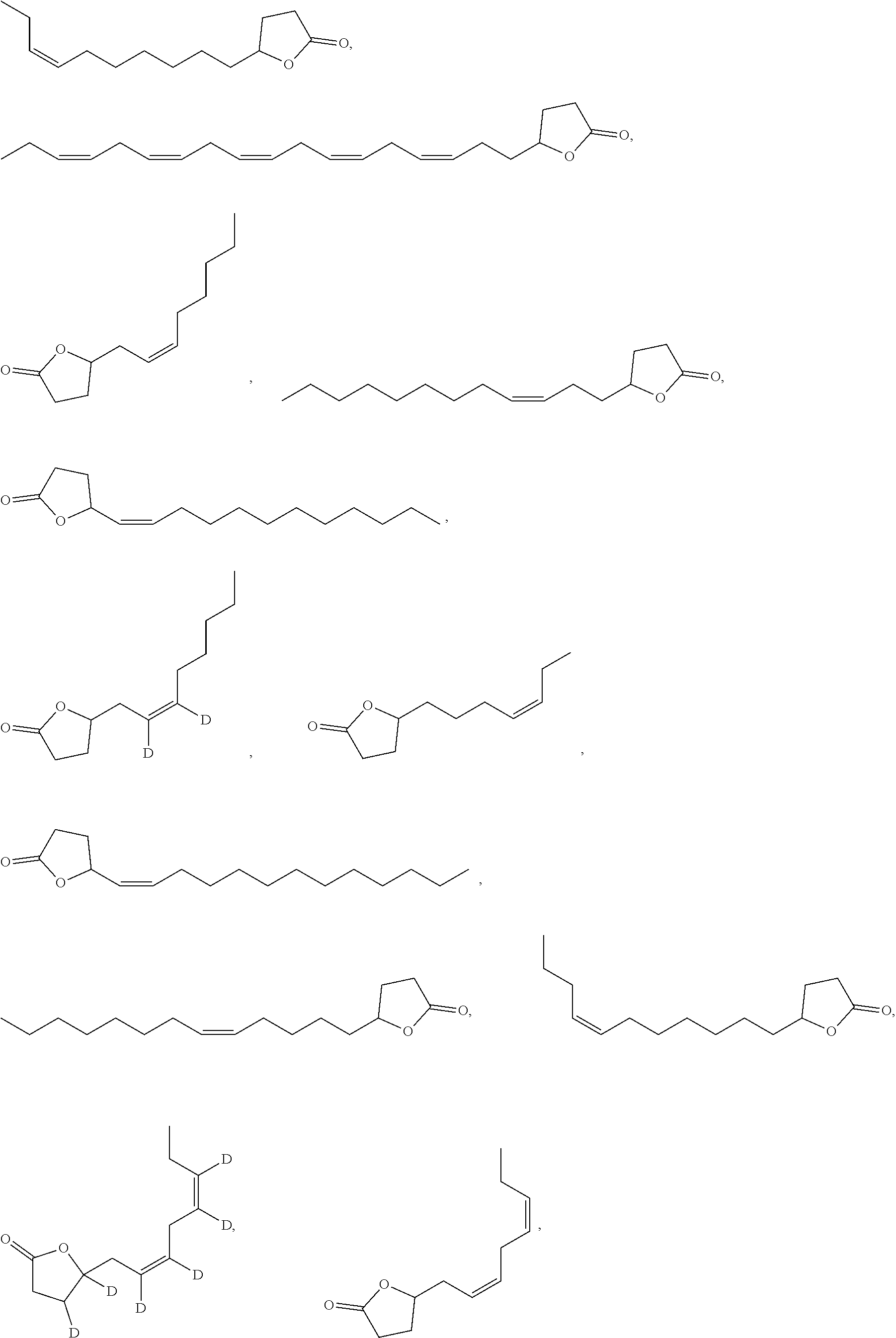

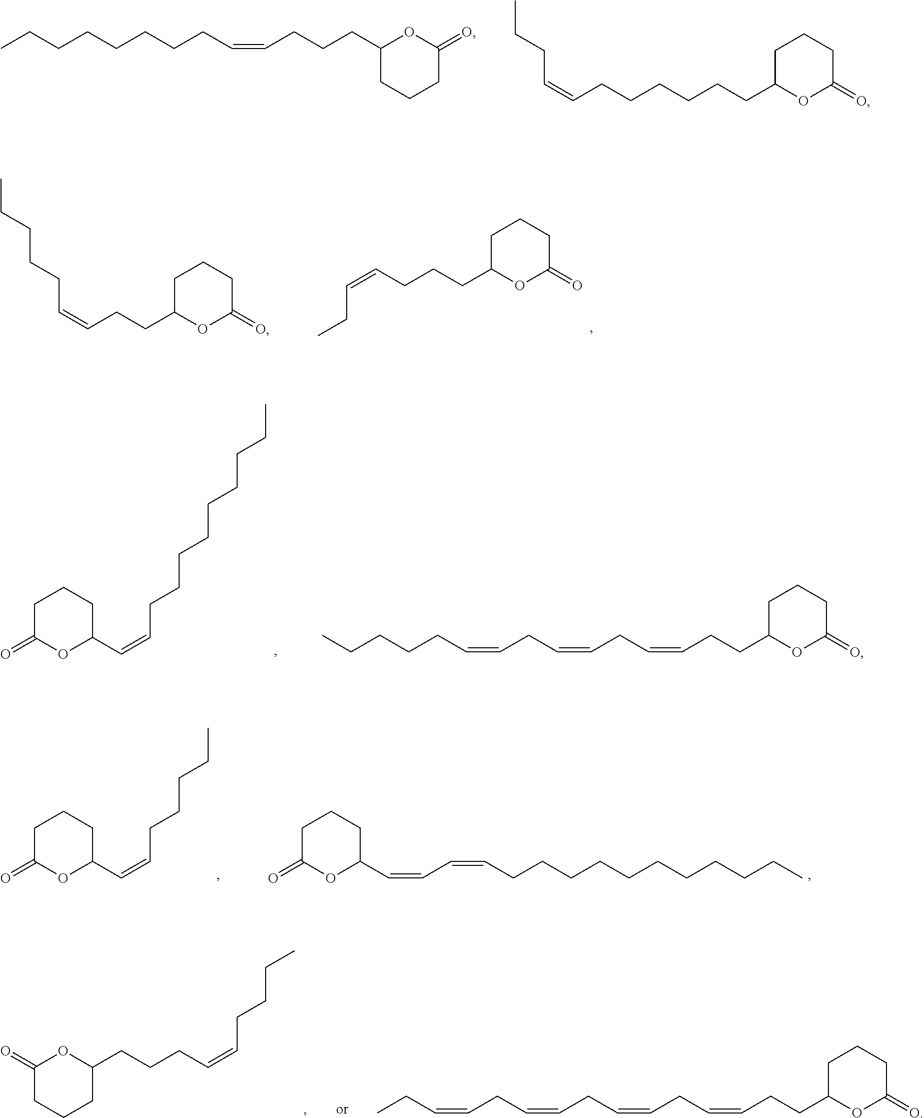

49. The gamma- or delta-lactone of embodiment 48, or a tautomer, isotopically labeled compound, solvate, polymorph, or co-crystal thereof, wherein R¹ comprises only one double bond.
50. The gamma- or delta-lactone of embodiment 48, or a tautomer, isotopically labeled compound, solvate, polymorph, or co-crystal thereof, wherein R¹ comprises only two double bonds.
51. The gamma- or delta-lactone of embodiment 48, or a tautomer, isotopically labeled compound, solvate, polymorph, or co-crystal thereof, wherein R¹ comprises only three double bonds.
52. The gamma- or delta-lactone of embodiment 48, or a tautomer, isotopically labeled compound, solvate, polymorph, or co-crystal thereof, wherein R¹ comprises only four double bonds.
53. The gamma-lactone of any one of embodiments 48-52, or a tautomer, isotopically labeled compound, solvate, polymorph, or co-crystal thereof, wherein n is 1.
54. The gamma-lactone of embodiment 48 represented by the formula:

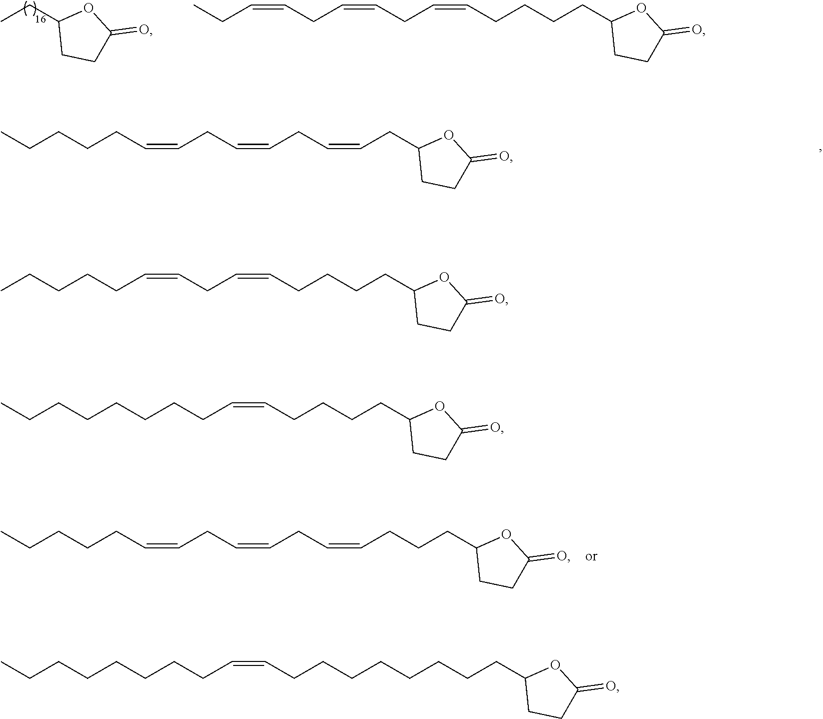

or a tautomer, isotopically labeled compound, solvate, polymorph, or co-crystal thereof.
   55. The delta-lactone of any one of embodiments 48-52, or a tautomer, isotopically labeled compound, solvate, polymorph, or co-crystal thereof, wherein n is 2.
   56. The delta-lactone of embodiment 48 represented by the formula:

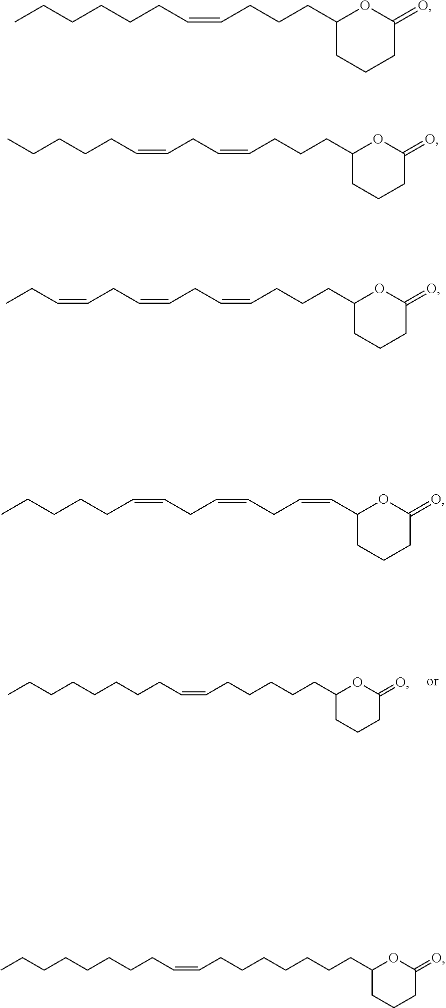

or a tautomer, isotopically labeled compound, solvate, polymorph, or co-crystal thereof.
   57. The bioconversion method of any one of embodiments 30-47, the bioenzymatic method of any one of embodiments 30-47, the gamma- or delta-lactone of any one of embodiments 48-56, or a tautomer, isotopically labeled compound, solvate, polymorph, or co-crystal thereof, wherein the chiral carbon atom is of the S configuration.
   58. The bioconversion method of any one of embodiments 30-47, the bioenzymatic method of any one of embodiments 30-47, the gamma- or delta-lactone of any one of embodiments 48-56, or a tautomer, isotopically labeled compound, solvate, polymorph, or co-crystal thereof, wherein the chiral carbon atom is of the R configuration.
   59. A delta-lactone, or a tautomer, isotopically labeled compound, solvate, polymorph, or co-crystal thereof, produced by the bioconversion method of any one of embodiments 1-2, 4-5, 7-12, 14-18, 27-33, 35-46, and 57-58.
   60. A delta-lactone, or a tautomer, isotopically labeled compound, solvate, polymorph, or co-crystal thereof, produced by the bioenzymatic method of any one of embodiments 25, 27-33, 35-46, and 57-58.
   61. A gamma-lactone, or a tautomer, isotopically labeled compound, solvate, polymorph, or co-crystal thereof, produced by the bioconversion method of any one of embodiments 3-4, 6-12, 14-18, 26-29, 34-45, 47, and 57-58.
   62. A gamma-lactone, or a tautomer, isotopically labeled compound, solvate, polymorph, or co-crystal thereof, produced by the bioenzymatic method of any one of embodiments 26-29, 34-45, 47, and 57-58.
   63. A mixture of two or more lactones, wherein each lactone is independently the gamma-or delta-lactone of any one of embodiments 48-62, or tautomer, isotopically labeled compound, solvate, polymorph, or co-crystal thereof.
   64. The bioconversion method of any one of embodiments 1-2, 4-5, 7-12, 14-18, 27-33, 35-46, and 57-58 or bioenzymatic method of any one of embodiments 25, 27-33, 35-46, and 57-58, wherein the product delta-lactone is the delta-lactone of any one of embodiments 48-52 and 55-60, or a tautomer, isotopically labeled compound, solvate, polymorph, or co-crystal thereof, or the mixture of embodiment 63.
   65. The bioconversion method of any one of embodiments 3-4, 6-12, 14-18, 26-29, 34-45, 47, and 57-58 or bioenzymatic method of any one of embodiments 26-29, 34-45, 47, and 57-58, wherein the product gamma-lactone is the gamma-lactone of any one of embodiments 48-54, 57-58, and 61-62, or a tautomer, isotopically labeled compound, solvate, polymorph, or co-crystal thereof, or the mixture of embodiment 63.
   66. The bioconversion method of any one of embodiments 1-2, 4-5, 7-12, 14-18, 27-33, 35-46, 57-58, and 64 or bioenzymatic method of any one of embodiments 25, 27-33, 35-46, 57-58 and 64, wherein the product delta-lactone has a purity of not less than 70%.
   67. The bioconversion method of embodiment 66 or bioenzymatic method of embodiment 66, wherein the product delta-lactone has a purity of not less than 75%.
   68. The bioconversion method of any one of embodiments 3-4, 6-12, 14-18, 26-29, 34-45, 47, 57-58, and 65 or bioenzymatic method of any one of embodiments 26-29, 34-45, 47, 57-58, and 65, wherein the product gamma-lactone has a purity of not less than 70%, optionally wherein the product gamma-lactone has a purity of not less than 75%.
   69. A composition comprising the product delta-lactone recited in any one of embodiments 1-2, 4-5, 7-12, 14-18, 25, 27-33, 35-46, 57-58, 64, and 66-67, or a tautomer, isotopically labeled compound, solvate, polymorph, or co-crystal thereof, the product gamma-lactone recited in any one of embodiments 3-4, 6-12, 14-18, 26-29, 34-45, 47, 57-58, 65, and 68, or a tautomer, isotopically labeled compound, solvate, polymorph, or co-crystal thereof, the gamma- or delta-lactone of any one of embodiments 48-62, or a tautomer, isotopically labeled compound, solvate, polymorph, or co-crystal thereof, or the mixture of embodiment 63.
   70. The composition of embodiment 69 further comprising a pharmaceutically acceptable excipient, cosmetically acceptable excipient, or nutraceutically acceptable excipient.
   71. A kit comprising:
the product delta-lactone recited in any one of embodiments 1-2, 4-5, 7-12, 14-18, 25, 27-33, 35-46, 57-58, 64, and 66-67, or a tautomer, isotopically labeled compound, solvate, polymorph, or co-crystal thereof, the product gamma-lactone recited in any one of embodiments 3-4, 6-12, 14-18, 26-29, 34-45, 47, 57-58, 65, and 68, or a tautomer, isotopically labeled compound, solvate, polymorph, or co-crystal thereof, the gamma- or delta-lactone of any one of embodiments 48-62, or a tautomer, isotopically labeled compound, solvate, polymorph, or co-crystal thereof, the mixture of embodiment 63, or the composition of any one of embodiments 69-70; and
instructions for using the product delta-lactone, or a tautomer, isotopically labeled compound, solvate, polymorph, or co-crystal thereof, the product gamma-lactone, or a tautomer, isotopically labeled compound, solvate, polymorph, or co-crystal thereof, the gamma- or delta-lactone, or a tautomer, isotopically labeled compound, solvate, polymorph, or co-crystal thereof, the mixture, or the composition.
   72. The bioconversion method of any one of embodiments 1-12, 14-18, 27-47, 57-58, and 64-68, the bioenzymatic method of any one of embodiments 25-47, 57-58, and 64-68, the gamma- or delta-lactone of any one of embodiments 48-62, or a tautomer, isotopically labeled compound, solvate, polymorph, or co-crystal thereof, the mixture of embodiment 63, the composition of any one of embodiments 69-70, the kit of embodiment 71, wherein the product delta-lactone, or a tautomer, isotopically labeled compound, solvate, polymorph, or co-crystal thereof, is the product delta-lactone; the product gamma-lactone, or a tautomer, isotopically labeled compound, solvate, polymorph, or co-crystal thereof, is the product gamma-lactone; the delta-lactone, or a tautomer, isotopically labeled compound, solvate, polymorph, or co-crystal thereof, is the delta-lactone; and the gamma-lactone, or a tautomer, isotopically labeled compound, solvate, polymorph, or co-crystal thereof, is the gamma-lactone.

1. A method for the production of a delta-lactone, the method comprising:
growing a cellular system in a culture medium, wherein the cellular system comprises a host cell which has been modified to express a recombinant cytochrome P450 hydroxylase polypeptide comprising one or more amino acid substitutions at positions N86, S272 and S341 in SEQ ID NO: 1;
expressing the recombinant cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof, thereby producing the delta-lactone in a recoverable amount; optionally wherein:
incubating the recombinant cytochrome P450 hydroxylase polypeptide comprising one or more amino acid substitutions at positions N86, S272 and S341 in SEQ ID NO: 1, with the substrate, wherein the substrate is a carboxylic acid, and NADPH for a sufficient time to convert the substrate to a hydroxylated fatty acid composition comprising one or more hydroxylated fatty acids, wherein a delta-hydroxylated fatty acid is present at a ratio of at least 20% of all hydroxylated fatty acids present in the hydroxylated fatty acid composition; and
acidifying the hydroxylated fatty acid composition to convert the delta-hydroxylated fatty acid to a delta-lactone.

2.-4. (canceled)

5. The method of claim 1, wherein the substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof

6. The method of claim 1, wherein the fatty acid substrate is represented by Formula (I): 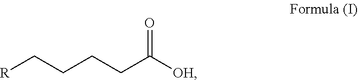
Wherein R is a C₁₋₂₀ alkyl group, a C₁₋₂₀ alkenyl, or a C₁₋₂₀ alkynyl group, or further wherein the delta-hydroxylated fatty acid is represented by Formula (II): 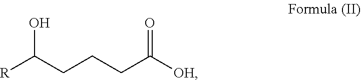
and the delta-lactone is represented by Formula (III): 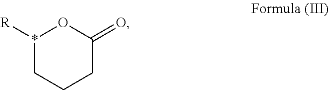
wherein R is a C₁₋₂₀ alkyl group, a C₁₋₂₀ alkenyl group, or a C₁₋₂₀ alkynyl group, and wherein * indicates a chiral carbon;
wherein R does not comprise a double bond;
wherein R comprises one, two, three, or four double bonds;
wherein each double bond is a Z double bond;
wherein the delta-lactones do not comprise C═C═C, and/or do not comprise C≡C.

7.-12. (canceled)

13. The method of claim 1, wherein the substrate is a carboxylic acid comprising a linear alkyl, alkenyl, or alkynyl moiety comprising twenty to twenty-two carbon atoms.

14. The method of claim 1, wherein the delta-lactone is of the formula: 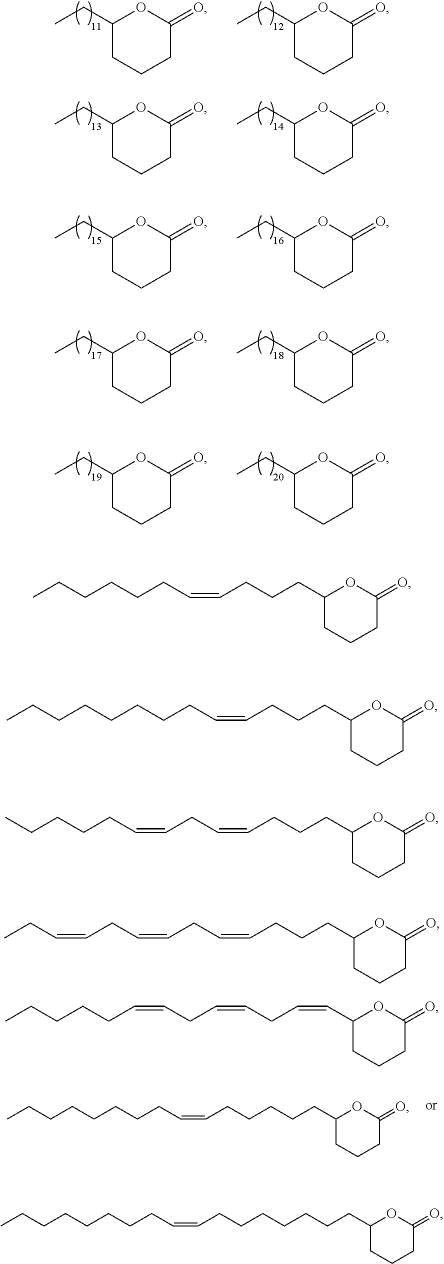
or a tautomer, isotopically labeled compound, solvate, polymorph, or co-crystal thereof; or a mixture thereof.

15.-16. (canceled)

17. The method of claim 1, wherein the recombinant cytochrome P450 hydroxylase polypeptide comprises an amino acid substitution selected from the group consisting of S272I, S272L, S272M, S272N, S272T, N276T, S272N/N86E, S272N/N86M, S272N/S341G, S272N/S341H, S272N/S341N, S272T/N86F, S272T/N86I, S272T/N86V, S272N/N86M/S341D, S272N/N86M/S341H, S272T/N86F/S341A, S272T/N86F/S341C, S272T/N86F/S341H, S272T/N86F/S341M, and S272T/N86F/S341Q.

18.-19. (canceled)

20. The method of claim 1, wherein the recombinant cytochrome P450 hydroxylase polypeptide comprises an amino acid sequence at least 90% identical to, or comprises, any one of SEQ ID NOs: 3, 5, 7, 9, or 11.

21. (canceled)

22. The method of claim 1, wherein said host cell is a bacterium, a yeast cell, a fungal cell, an alga cell, a plant cell,
a bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium,
a fungus of a genus selected from the group consisting of Saccharomyces: Zygosaccharomyces; Kluyveromyces; Candida; Streptomyces; Hansenula; Debaryomyces; Mucor; Pichia; Torulopsis; Aspergillus; and Arthrobotlys,
or wherein the host cell is E. coli.

23.-25. (canceled)

26. The method of claim 1, wherein the delta-lactone has a purity of not less than 50%, or not less than 75%.

27. (canceled)

28. A delta-lactone, or a tautomer, isotopically labeled compound, solvate, polymorph, or co-crystal thereof, produced by the method of claim 1.

29.-31. (canceled)

32. A method for the production of a gamma-lactone, the method comprising:
growing a cellular system in a culture medium, wherein the cellular system comprises a host cell which has been modified to express a recombinant cytochrome P450 hydroxylase polypeptide comprising an amino acid sequence that is at least 80% identical to SEQ ID NO: 1;
expressing the recombinant cytochrome P450 hydroxylase polypeptide in the cellular system;
exposing the cellular system to a substrate and NADPH, wherein said substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof, thereby producing the gamma-lactone in a recoverable amount,
optionally wherein the carboxylic acid comprising a linear or branched, alkyl, alkenyl, or alkynyl moiety comprising twenty-one to twenty-five carbon atoms; further optionally wherein:
incubating the recombinant cytochrome P450 hydroxylase polypeptide comprising an amino acid sequence that is at least 80% identical to SEQ ID NO: 1, with the substrate, wherein the substrate is a carboxylic acid, and NADPH for a sufficient time to convert the substrate to a hydroxylated fatty acid composition comprising one or more hydroxylated fatty acids, wherein a gamma-hydroxylated fatty acid is present at a ratio of at least 20% of all hydroxylated fatty acids present in the hydroxylated fatty acid composition; and
acidifying the hydroxylated fatty acid composition to convert the gamma-hydroxylated fatty acid to a gamma-lactone.

33.-35. (canceled)

36. The method of claim 32, wherein the substrate is a carboxylic acid comprising a linear or branched, alkyl, alkenyl, or alkynyl moiety comprising sixteen to twenty-five carbon atoms, a salt thereof, an alkyl ester thereof, a mono, di or triglyceride thereof or an unsubstituted monoalkyl or dialkyl amide thereof, optionally wherein the carboxylic acid comprising a linear or branched, alkyl, alkenyl, or alkynyl moiety comprising twenty-one to twenty-five carbon atoms.

37. The method of claim 32, wherein the gamma-lactone is represented by Formula (IV): 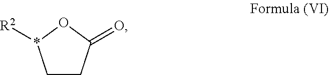
wherein R² is a C₁₂₋₂₁ alkyl group, a C₁₂₋₂₁ alkenyl group, or a C₁₂₋₂₁ alkynyl group, and wherein * indicates a chiral carbon;
wherein R² is does not comprise a double bond;
wherein R² comprises one, two, three, or four double bonds;
wherein each double bond is a Z double bond;
wherein the gamma-lactones do not comprise C═C═C, and/or do not comprise C≡C.

38.-42. (canceled)

43. The method of claim 32, wherein the substrate is a carboxylic acid comprising a linear alkyl, alkenyl, or alkynyl moiety comprising twenty to twenty-two carbon atoms.

44. The method of claim 32, wherein the gamma-lactone is of the formula: 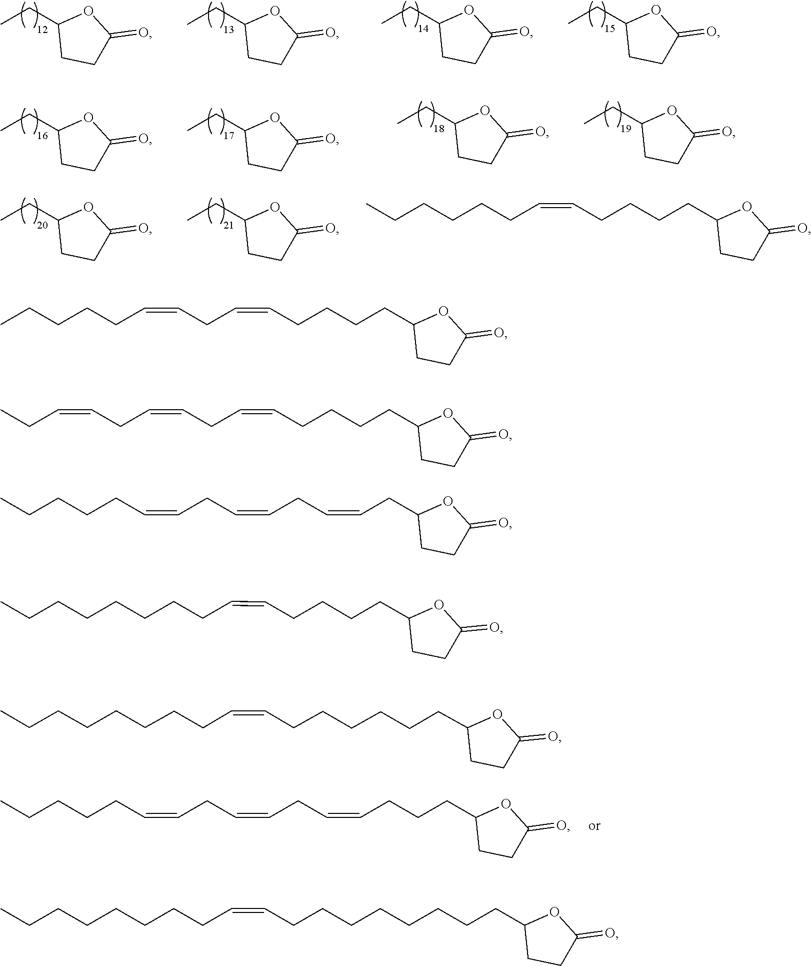
or a tautomer, isotopically labeled compound, solvate, polymorph, or co-crystal thereof; or a mixture thereof.

45.-46. (canceled)

47. The method of claim 32, wherein the recombinant cytochrome P450 hydroxylase polypeptide comprises an amino acid sequence at least 90% identical to, or comprises, the amino acid sequence of SEQ ID NO: 1.

48. (canceled)

49. The method of a claim 32, wherein said host cell is a bacterium, a yeast cell, a fungal cell, an alga cell, or a plant cell, optionally wherein:
the host cell is bacterial cell of a genus selected from the group consisting of Escherichia; Salmonella; Bacillus; Acinetobacter; Corynebacterium; Methylosinus; Methylomonas; Rhodococcus; Pseudomonas; Rhodobacter; Synechocystis; Brevibacteria; Microbacterium; Arthrobacter; Citrobacter; Escherichia; Klebsiella; Pantoea; Salmonella; Corynebacterium; and Clostridium;
the host cell is a fungus of a genus selected from the group consisting of Saccharomyces; Zygosaccharomyces; Kluyveromyces; Candida; Streptomyces; Hansenula; Debaryomyces; Mucor; Pichia; Torulopsis; Aspergillus; and Arthrobotlys;
the host cell is E. coli.

50.-52. (canceled)

53. The method of claim 32, wherein the gamma-lactone has a purity of not less than 50%, or not less than 75%.

54. (canceled)

55. A gamma-lactone, or a tautomer, isotopically labeled compound, solvate, polymorph, or co-crystal thereof, produced by the method of claim 32.

56.-58. (canceled)

59. A delta lactone represented by the formula: 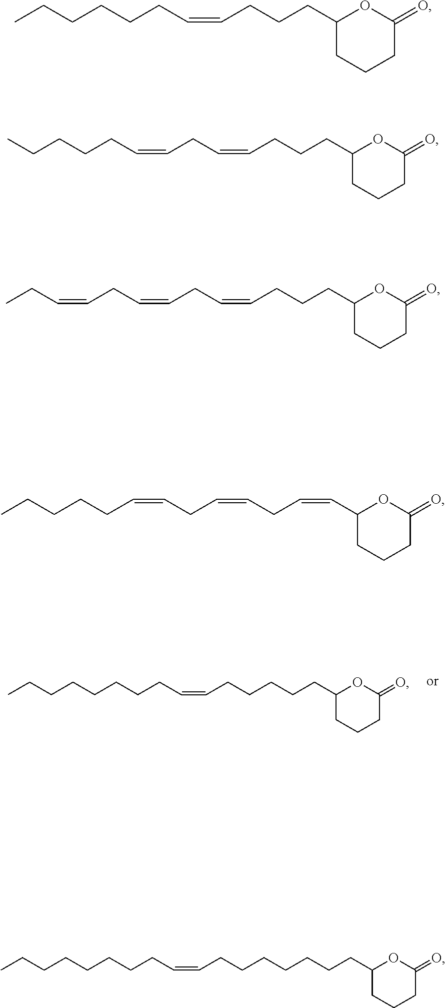
or a tautomer, isotopically labeled compound, solvate, polymorph, or co-crystal thereof;
and/or a gamma lactone represented by the formula: 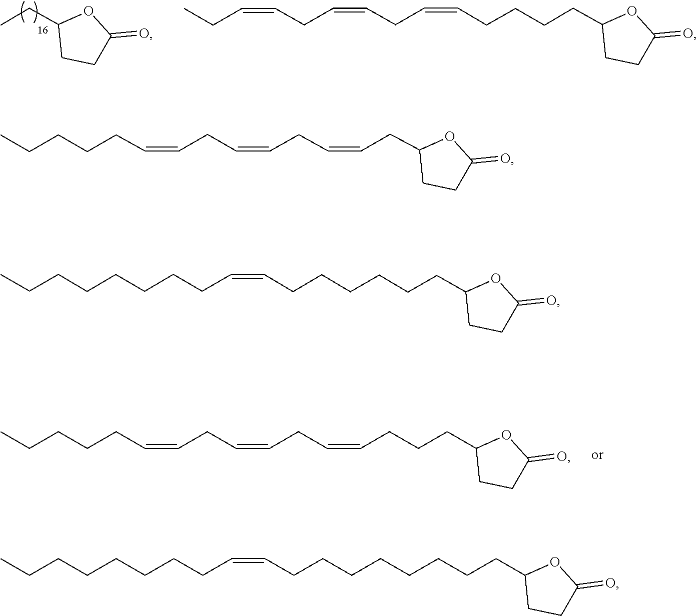
or a tautomer, isotopically labeled compound, solvate, polymorph, or co-crystal thereof.

60.-62. (canceled)
